(19) United States
(12) Patent Application Publication
(10) Pub. No.: US 2019/0329042 A1
(43) Pub. Date: Oct. 31, 2019

(54) METHOD, APPARATUS, SURGICAL TECHNIQUE, AND OPTIMAL STIMULATION PARAMETERS FOR NONINVASIVE & MINIMALLY INVASIVE AUTONOMIC VECTOR NEUROMODULATION FOR PHYSIOLOGIC OPTIMIZATION AND FOR THE TREATMENT OF OBESITY, CARDIAC DISEASE, PULMONARY DISORDERS, HYPERTENSION, AND OTHER CONDITIONS

(71) Applicant: Daniel John DiLorenzo, Loma Linda, CA (US)

(72) Inventor: Daniel John DiLorenzo, Loma Linda, CA (US)

(21) Appl. No.: 16/286,570

(22) Filed: Feb. 26, 2019

Related U.S. Application Data
(63) Continuation of application No. 62/635,467, filed on Feb. 26, 2018, now abandoned.
Application No. 62/635,467 is a continuation of application No. 62/780,332, filed on Dec. 17, 2018.

(51) Int. Cl. A61N 1/36; A61N 1/372

(52) U.S. Cl. CPC A61N 1/36189; A61N 1/3727; A61N 1/36062; A61N 1/36067; A61N 1/36146; G16H 20/30; A61N 1/36114; A61N 1/36071; A61N 1/36007; A61N 1/36085; A61N 1/36103

(57) ABSTRACT

The present invention teaches a method and apparatus for physiological modulation, including neural, gastrointestinal, renal, respiratory, and other modulation, for the purposes of treating several disorders, including obesity, depression, epilepsy, diabetes, hypertension, asthma, and other disorders. This includes implanted, percutaneous, hybrid implanted and nonimplanted, nonimplanted, noninvasive neural and neuromuscular modulators, used to deliver autonomic vector modulation to deliver optimal therapy via coordinated multi-nodal modulation at least one of the afferent and efferent neurons of the sympathetic and parasympathetic nervous systems and other nervous system pathways.

### RELATED APPLICATIONS

All patents and patent applications listed are hereby incorporated by reference.

This application incorporates by reference U.S. Provisional Patent Application Ser. No. 61/919,835 (Docket GIPROG 06.01p1), filed Dec. 23, 2013, entitled “METHOD, APPARATUS, SURGICAL TECHNIQUE, AND OPTIMAL STIMULATION PARAMETERS FOR NONINVASIVE & MINIMALLY INVASIVE AUTONOMIC NEUROMODULATION FOR THE TREATMENT OF OBESITY, PULMONARY DISORDERS, HYPERTENSION, AND OTHER CONDITIONS”, and naming as inventor Daniel John DiLorenzo, which is incorporated by reference.

This application incorporates by reference U.S. Provisional Patent Application Ser. No. 62/569,975 (Docket GIPROG 06.01p2), filed Oct. 9, 2017, entitled “METHOD, APPARATUS, SURGICAL TECHNIQUE, AND OPTIMAL STIMULATION PARAMETERS FOR NONINVASIVE & MINIMALLY INVASIVE AUTONOMIC VECTOR NEUROMODULATION FOR THE TREATMENT OF OBESITY, PULMONARY DISORDERS, HYPERTENSION, AND OTHER CONDITIONS”, and naming as inventor Daniel John DiLorenzo, which is incorporated by reference.

This application is a continuation of and incorporates by reference U.S. Provisional Patent Application Ser. No. 62/635,467 (Docket GIPROG 06.01p3), filed Feb. 28, 2018, entitled “METHOD, APPARATUS, SURGICAL TECHNIQUE, AND OPTIMAL STIMULATION PARAMETERS FOR NONINVASIVE & MINIMALLY INVASIVE AUTONOMIC VECTOR NEUROMODULATION FOR THE TREATMENT OF OBESITY, PULMONARY DISORDERS, HYPERTENSION, AND OTHER CONDITIONS”, and naming as inventor Daniel John DiLorenzo, which is incorporated by reference.

This application is a continuation of and incorporates by reference U.S. Provisional Patent Application Ser. No. 62/780,332 (Docket GIPROG 06.01p4), filed Dec. 17, 2018, entitled “METHOD, APPARATUS, SURGICAL TECHNIQUE, AND OPTIMAL STIMULATION PARAMETERS FOR NONINVASIVE & MINIMALLY INVASIVE AUTONOMIC VECTOR NEUROMODULATION FOR THE TREATMENT OF OBESITY, PULMONARY DISORDERS, HYPERTENSION, AND OTHER CONDITIONS”, and naming as inventor Daniel John DiLorenzo, which is incorporated by reference.

This application incorporates by reference U.S. patent application Ser. No. 12/317,448 (Docket GIELEC 01.01), filed Dec. 22, 2008, entitled “METHOD AND APPARATUS FOR CONFORMAL ELECTRODES FOR AUTONOMIC NEUROMODULATION FOR THE TREATMENT OF OBESITY AND OTHER CONDITIONS”, which is a continuation of U.S. Provisional Patent Application Ser. No. 61/008,804 (Docket GIELEC 01.01p1), filed Dec. 21, 2007, entitled “METHOD AND APPARATUS FOR CONFORMAL ELECTRODES FOR AUTONOMIC NEUROMODULATION FOR THE TREATMENT OF OBESITY AND OTHER CONDITIONS”, and which is a continuation in Part of U.S. patent application Ser. No. 12/291,685 (Docket GIPROG 01.01), filed Nov. 12, 2008, entitled “METHOD AND APPARATUS FOR PROGRAMMING OF AUTONOMIC NEUROMODULATION FOR THE TREATMENT OF OBESITY”, which claims priority to U.S. Provisional Patent Application Ser. No. 61/002,735, filed Nov. 12, 2007, entitled “METHOD AND APPARATUS FOR PROGRAMMING OF AUTONOMIC NEUROMODULATION FOR THE TREATMENT OF OBESITY” and U.S. Provisional Patent Application Ser. No. 61/003,686, filed Nov. 19, 2007, entitled “METHOD AND APPARATUS FOR OPERATION OF AUTONOMIC NEUROMODULATION FOR THE TREATMENT OF OBESITY”, all naming as inventor Daniel John DiLorenzo, and all of which are incorporated by reference.

This application incorporates by reference U.S. patent application Ser. No. 12/291,685 (Docket GIPROG 01.01), filed Nov. 12, 2008, entitled “METHOD AND APPARATUS FOR PROGRAMMING OF AUTONOMIC NEUROMODULATION FOR THE TREATMENT OF OBESITY”, which names as inventor Daniel John DiLorenzo, which is incorporated by reference.

This application incorporates by reference U.S. patent application Ser. No. 11/716,451 (Docket GISTIM 03.01), entitled METHOD, APPARATUS, SURGICAL TECHNIQUE, AND STIMULATION PARAMETERS FOR AUTONOMIC NEUROMODULATION FOR THE TREATMENT OF OBESITY, filed Mar. 9, 2007, which names as inventor Daniel John DiLorenzo, and which is incorporated by reference, which is a continuation of and claims the benefit of U.S. Provisional Patent Application Ser. No. 60/786,250 Filed Mar. 27, 2006.

This application incorporates by reference U.S. patent application Ser. No. 11/317,099 (Docket GISTIM 02.02), filed Dec. 22, 2005, entitled “METHOD, APPARATUS, AND SURGICAL TECHNIQUE FOR AUTONOMIC NEUROMODULATION FOR THE TREATMENT OF OBESITY”, which names as inventor Daniel John DiLorenzo.

This application incorporates by reference U.S. patent application Ser. No. 12/462,903 (Docket GISTIM 01.03), filed Aug. 9, 2009, entitled “Methods and Apparatus for Neuromodulation and Physiologic Modulation for the Treatment of Obesity and Metabolic and Neuropsychiatric Disease”, which is a continuation of U.S. patent application Ser. No. 12/387,638 (Docket GISTIM 01.04), filed May 5, 2009, entitled “Methods and Apparatus for Neuromodulation and Physiologic Modulation for the Treatment of Obesity and Metabolic and Neuropsychiatric Disease”, which is a continuation of U.S. patent application Ser. No. 10/982,549 (Docket GISTIM 01.02), now U.S. Pat. No. 7,529,582, filed Jun. 21, 2004, entitled “Method And Apparatus For Neuromodulation And Physiologic Modulation For The Treatment Of Metabolic And Neuropsychiatric Disease”, which is a continuation of U.S. patent application Ser. No. 10/198,871 (Docket GISTIM 01.01), filed Jul. 19, 2002, now U.S. Pat. No. 7,599,736, entitled “Method and Apparatus for the Neuromodulation and Physiologic Modulation for the Treatment of Metabolic and Neuropsychiatric Disease”, which is a continuation of U.S. Provisional Patent Application No. 60/307,124, filed Jul. 19, 2001, entitled “Physiologic Modulation for the Control of Obesity, Depression, Epilepsy, and Diabetes”, all naming as inventor, Daniel John DiLorenzo.

This application incorporates by reference U.S. patent application Ser. No. 11/187,315, now U.S. Pat. No. 7,974,696 (Docket ANSTIM 01.01) entitled “CLOSED-LOOP AUTONOMIC NEUROMODULATION FOR OPTIMAL CONTROL OF NEUROLOGICAL AND METABOLIC DISEASE”, filed Jul. 23, 2005, issued Jul. 5, 2011, and naming as inventor Daniel J. DiLorenzo.

This application incorporates by reference U.S. Pat. No. 6,366,813 B1 (Docket PDSTIM 01.01), entitled APPARATUS AND METHOD FOR CLOSED-LOOP INTRACRANIAL STIMULATION FOR OPTIMAL CONTROL OF NEUROLOGICAL DISEASE., filed Jun. 25, 1999, issued Apr. 2, 2002, and naming as inventor Daniel J. DiLorenzo.

This application incorporates by reference U.S. Pat. No. 6,819,956 B2, entitled OPTIMAL METHOD AND APPARATUS FOR NEURAL MODULATION FOR THE TREATMENT OF NEUROLOGICAL DISEASE, PARTICULARLY MOVEMENT DISORDERS., filed Nov. 11, 2001, issued Nov. 16, 2004, and naming as inventor Daniel J. DiLorenzo.

This application incorporates by reference U.S. Patent Application Number US 2005/0021104 A1, entitled APPARATUS AND METHOD FOR CLOSED-LOOP INTRACRANIAL STIMULATION FOR OPTIMAL CONTROL OF NEUROLOGICAL DISEASE., filed Apr. 5, 2004, and naming as inventor Daniel J. DiLorenzo.

This application incorporates by reference all Patent Applications naming as inventor Daniel J. DiLorenzo.

This application incorporates by reference all Patents naming as inventor Daniel J. DiLorenzo.

This application incorporates by reference all patents and non-patent documents and references cited in the specification and in the information disclosure statements.

### BACKGROUND OF THE INVENTION

### Field of the Invention

The present invention relates generally to metabolic disease and neuropsychiatric disease and, more particularly, to stimulation of gastric and autonomic including sympathetic and parasympathetic neural tissue for the treatment of disease, including but not limited to obesity, eating disorders including anorexia and bulimia, depression, anxiety, epilepsy, metabolic conditions, diabetes, hyperglycemia, hypoglycemia, irritable bowel syndrome, immunological conditions, asthma, respiratory conditions, cardiovascular conditions, cardiac conditions, vascular conditions, headaches, substance abuse, substance addiction, smoking cessation, drug withdrawal, hyperhidrosis, reflex sympathetic dystrophy, pain, and other medical and neurological and psychiatric conditions.

### Related Art

Physiologic studies have demonstrated the presence of a sympathetic nervous system afferent pathway transmitting gastric distention information to the hypothalamus. [Barone, Zarco de Coronado et al. (1995). Gastric distension modulates hypothalamic neurons via a sympathetic afferent path through the mesencephalic periaqueductal gray. Brain Research Bulletin. 38: 239-51.] However, prior techniques have generally not addressed the problems associated with satiety, morbidity, mortality of intracranial modulation and the risk of ulcers. Unlike prior techniques, by specifically targeting sympathetic afferent fibers, the present invention effects the sensation of satiety and avoids the substantial risks of morbidity and mortality of intracranial modulation, particularly dangerous in the vicinity of the hypothalamus. Furthermore, this invention avoids the risk of ulcers inherent in vagus nerve stimulation.

A. Satiety. Stimulation of intracranial structures has been proposed and described for the treatment of obesity (U.S. Pat. No. 5,782,798). Stimulation of the left ventromedial hypothalamic (VMH) nucleus resulted in delayed eating by dogs who had been food deprived. Following 24 hours of food deprivation, dogs with VMH stimulation waited between 1 and 18 hours after food presentation before consuming a meal. Sham control dogs ate immediately upon food presentation. Dogs that received 1 hour of stimulation every 12 hours for 3 consecutive days maintained an average daily food intake of 35% of normal baseline levels. [Brown, Fessler et al. (1984). Changes in food intake with electrical stimulation of the ventromedial hypothalamus in dogs. Journal of Neurosurgery. 60: 1253-7.] B. Candidate Peripheral Nerve Pathways for Modulating Satiety.

B1. Sympathetic Afferents. The effect of gastric distension on activity in the lateral hypothalamus-lateral preoptic area-medial forebrain bundle (LPA-LH-MFB) was studied to determine the pathways for this gastric afferent input to the hypothalamus. [Barone, Zarco de Coronado et al. (1995). Gastric distension modulates hypothalamic neurons via a sympathetic afferent path through the mesencephalic periaqueductal gray. Brain Research Bulletin. 38: 239-51.] The periaqueductal gray matter (PAG) was found to be a relay station for this information. [Barone, Zarco de Coronado et al. (1995). Gastric distension modulates hypothalamic neurons via a sympathetic afferent path through the mesencephalic periaqueductal gray. Brain Research Bulletin. 38: 239-51.] This modulation of the hypothalamus was attenuated but not permanently eliminated by bilateral transection of the vagus nerve. This modulation was, however, significantly reduced or eliminated by bilateral transection of the cervical sympathetic chain or spinal transection at the first cervical level. [Barone, Zarco de Coronado et al. (1995). Gastric distension modulates hypothalamic neurons via a sympathetic afferent path through the mesencephalic periaqueductal gray. Brain Research Bulletin. 38: 239-51.] These signals containing gastric distension and temperature stimulation are mediated to a large degree by sympathetic afferents, and the PAG is a relay station for this gastric afferent input to the hypothalamus. [Barone, Zarco de Coronado et al. (1995). Gastric distension modulates hypothalamic neurons via a sympathetic afferent path through the mesencephalic periaqueductal gray. Brain Research Bulletin. 38: 239-51.] For example, in the LPA-LH-MFB study, 26.1% of the 245 neurons studied were affected by gastric stimulation, with 17.6% increasing in firing frequency and 8.6% decreasing during gastric distension. [Barone, Zarco de Coronado et al. (1995). Gastric distension modulates hypothalamic neurons via a sympathetic afferent path through the mesencephalic periaqueductal gray. Brain Research Bulletin. 38: 239-51.] The response of 8 of 8 neurons sensitive to gastric distension were maintained, though attenuated after bilateral vagus nerves were cut. In 2 of these 8 cells, the effect was transiently eliminated for 2-4 minutes after left vagus transection, and then activity recovered. In 3 LH-MFB cells, two increased and the other decreased firing rate with gastric distension. Following bilateral sympathetic ganglion transection, the response of two were eliminated, and the third (which increased firing with distension) had a significantly attenuated response. [Barone, Zarco de Coronado et al. (1995). Gastric distension modulates hypothalamic neurons via a sympathetic afferent path through the mesencephalic periaqueductal gray. Brain Research Bulletin. 38: 239-51.] Vagus stimulation resulted in opposite or similar responses as gastric distension on the mesencephalic cells.

B2. Vagus Nerve Afferents. Gastric vagal input to neurons throughout the hypothalamus has been characterized. [Yuan and Barber (1992). Hypothalamic unitary responses to gastric vagal input from the proximal stomach. American Journal of Physiology. 262: G74-80.] Nonselective epineural vagus nerve stimulation (VNS) has been described for the treatment of Obesity (U.S. Pat. No. 5,188,104). This suffers from several significant limitations that are overcome by the present invention.

The vagus nerve is well known to mediate gastric hydrochloric acid secretion. Dissection of the vagus nerve off the stomach is often performed as part of major gastric surgery for ulcers. Stimulation of the vagus nerve may pose risks for ulcers in patients, of particular concern, as obese patients often have gastroesophageal reflux disease (GERD); further augmentation of gastric acid secretion would only exacerbate this condition.

C. Assessment of Sympathetic and Vagus Stimulation. The present invention teaches a significantly more advanced neuroelectric interface technology to stimulate the vagus nerve and avoid the efferent vagus side effects, including speech and cardiac side effects common in with existing VNS technology as well as the potential ulcerogenic side effects. However, since sympathetic afferent activity appears more responsive to gastric distension, this may represent a stronger channel for modulating satiety. Furthermore, by pacing stimulating modulators on the greater curvature of the stomach, one may stimulate the majority of the circular layer of gastric musculature, thereby diffusely increasing gastric tone.

D. Neuromuscular Stimulation. The muscular layer of the stomach is comprised of 3 layers: (1) an outer longitudinal layer, (2) a circular layer in between, and (3) a deeper oblique layer. [Gray (1974). Gray's Anatomy. T. Pick and R. Howden. Philadelphia, Running Press.] The circular fibers, which lie deep to the superficial longitudinal fibers, would appear to be the layer of choice for creating uniform and consistent gastric contraction with elevated wall tension and luminal pressure. Therefore, modulators should have the ability to deliver stimulation through the longitudinal layer. If the modulator is in the form of an electrode, then the electrodes should have the ability to deliver current through the longitudinal layer.

Gray's Anatomy describes innervation as including the right and left pneumogastric nerves (not the vagus nerves), being distributed on the back and front of the stomach, respectively. A great number of branches from the sympathetic nervous system also supply the stomach. [Gray (1974). Gray's Anatomy. T. Pick and R. Howden. Philadelphia, Running Press.] Metabolic Modulation (Efferent) Electrical stimulation of the VMH enhances lipogenesis in the brown adipose tissue (BAT), preferentially over the white adipose tissue (WAT) and liver, probably through a mechanism involving activation of the sympathetic innervation of the BAT. [Takahashi and Shimazu (1982). Hypothalamic regulation of lipid metabolism in the rat: effect of hypothalamic stimulation on lipogenesis. Journal of the Autonomic Nervous System. 6: 225-35.] The VMH is a hypothalamic component of the sympathetic nervous system. [Ban (1975). Fiber connections in the hypothalamus and some autonomic functions. Pharmacology, Biochemistry & Behavior. 3: 3-13.] A thermogenic response in BAT was observed with direct sympathetic nerve stimulation. [Flaim, Horwitz et al. (1977). Coupling of signals to brown fat: a- and b-adrenergic responses in intact rats. Amer. J. Physiol. 232: R101-R109.] The BAT had abundant sympathetic innervation with adrenergic fibers that form nest-like networks around every fat cell, [Derry, Schonabum et al. (1969). Two sympathetic nerve supplies to brown adipose tissue of the rat. Canad. J. Physiol. Pharmacol. 47: 57-63.] whereas WAT has no adrenergic fibers in direct contact with fat cells except those related to the blood vessels. [Daniel and Derry (1969). Criteria for differentiation of brown and white fat in the rat. Canad. J. Physiol. Pharmacol. 47: 941-945.]

### SUMMARY OF THE INVENTION

The present invention teaches apparatus and methods for treating a multiplicity of diseases, including obesity, depression, epilepsy, diabetes, and other diseases. The invention taught herein employs a variety of energy modalities to modulate central nervous system structures, peripheral nervous system structures, and peripheral tissues and to modulate physiology of neural structures and other organs, including gastrointestinal, adipose, pancreatic, and other tissues. The methods for performing this modulation, including the sites of stimulation and the modulator configurations are described. The apparatus for performing the stimulation are also described. This invention teaches a combination of novel anatomic approaches and apparatus designs for direct and indirect modulation of the autonomic nervous system, which is comprised of the sympathetic nervous system and the parasympathetic nervous system.

For the purposes of this description the term GastroPace™ should be interpreted to mean the devices constituting the system of the present embodiment of this invention, including the obesity application as well as others described, implied, enabled, facilitated, and derived from those taught in the present invention.

A. Obesity and Eating Disorders. The present invention teaches several mechanisms, including neural modulation and direct contraction of the gastric musculature, to effect the perception of satiety. This modulation is useful in the treatment of obesity and eating disorders, including anorexia nervosa and bulimia.

Direct stimulation of the gastric musculature increases the intraluminal pressure within the stomach; and this simulates the physiologic condition of having a full stomach, sensed by stretch receptors in the muscle tissue and transmitted via neural afferent pathways to the hypothalamus and other central nervous system structures, where the neural activity is perceived as satiety.

This may be accomplished with the several alternative devices and methods taught in the present invention. Stimulation of any of the gastric fundus, greater curvature of stomach, pyloric antrum, or lesser curvature of stomach, or other region of the stomach or gastrointestinal tract, increases the intraluminal pressure. Increase of intraluminal pressure physiologically resembles fullness of the respective organ, and satiety is perceived.

The present invention also includes the restriction of the flow of food to effect satiety. This is accomplished by stimulation of the pylorus. The pylorus is the sphincter-like muscle at the distal juncture of the stomach with the duodenum, and it regulates food outflow from the stomach into the duodenum. By stimulating contraction of the pylorus, food outflow from the stomach is slowed or delayed. The presence of a volume of food in the stomach distends the gastric musculature and causes the person to experience satiety.

B. Depression and Anxiety. An association has been made between depression and overeating, particularly with the craving of carbohydrates; and is believed to be an association between the sense of satiety and relief of depression. Stimulation of the gastric tissues, in a manner that resembles or is perceived as satiety, as described above, provides relief from this craving and thereby relief from some depressive symptoms. There are several mechanisms, including those taught above for the treatment of obesity that are applicable to the treatment of depression, anxiety, agoraphobia, social anxiety, panic attacks, and other neurological and psychiatric conditions.

An object of the present invention, as taught in the parent case, is the modulation of the autonomic nervous system for physiologic modulation, including modulation of limbic physiology, which has efficacy in the treatment of depression, anxiety and other psychiatric conditions. By altering the level of sympathetic nervous system activity, or the level of parasympathetic nervous system activity, or the ratio of sympathetic to parasympathetic nervous system activity (as reflected in metrics such as the autonomic index), the level of activity in the locus cemleus, solitary nucleus, cingulate nucleus, the limbic system, the supraorbital cortex, and other regions may be modulated, thereby influencing affect or mood as well as level of anxiety. Furthermore, the reduction of systemic sympathetic activity may be used to alleviate the symptoms of anxiety, which is employed in both the treatment of anxiety and in the conditioning of patients to control anxiety.

C. Epilepsy. The present invention includes electrical stimulation of peripheral nervous system and other structures and tissues to modulate the activity in the central nervous system to control seizure activity.

This modulation takes the form of peripheral nervous system stimulation using a multiplicity of novel techniques and apparatus. Direct stimulation of peripheral nerves is taught; this includes stimulation of the vagus, trigeminal, accessory, and sympathetic nerves. Indiscriminate stimulation of the vagus nerves has been described for some disorders, but the limitations in this technique are substantial, including cardiac rhythm disruptions, speech difficulties, and gastric and duodenal ulcers. The present invention overcomes these persistent limitations by teaching a method and apparatus for the selective stimulation of structures, including the vagus nerve as well as other peripheral nerves, and other neural, neuromuscular, and other tissues.

The present invention further includes noninvasive techniques for neural modulation. This includes the use of tactile stimulation to activate peripheral or cranial nerves. This noninvasive stimulation includes the use of tactile stimulation, including light touch, pressure, vibration, and other modalities that may be used to activate the peripheral or cranial nerves. Temperature stimulation, including hot and cold, as well as constant or variable temperatures, are included in the present invention.

D. Diabetes. The response of the gastrointestinal system, including the pancreas, to a meal includes several phases. The first phase, the anticipatory stage, is neurally mediated. Prior to the actual consumption of a meal, saliva production increases and the gastrointestinal system prepares for the digestion of the food to be ingested. Innervation of the pancreas, in an analogous manner, controls production of insulin.

Modulation of pancreatic production of insulin may be performed by modulation of at least one of afferent or efferent neural structures. Afferent modulation of at least one of the vagus nerve, the sympathetic structures innervating the gastrointestinal tissue, the sympathetic trunk, and the gastrointestinal tissues themselves is used as an input signal to influence central and peripheral nervous system control of insulin secretion.

E. Irritable bowel Syndrome. An object of the present invention, as taught in the parent case, is the modulation of the autonomic nervous system for physiologic modulation, including modulation of gastrointestinal physiology, which has efficacy in the treatment of irritable bowel syndrome. By altering the level of sympathetic nervous system activity, or the level of parasympathetic nervous system activity, or the ratio of sympathetic to parasympathetic nervous system activity (as reflected in metrics such as the autonomic index), the level of gastrointestinal motility and absorption may be modulated.

Modulation including down-regulation of the activity of the gastrointestinal tract, through autonomic modulation, as taught in the parent case has application to the treatment of irritable bowel syndrome. Said autonomic modulation includes but is not limited to inhibition or blocking of sympathetic nervous system activity and to enhancement or stimulation of parasympathetic nervous system activity.

The response of the gastrointestinal system to sympathetic stimulation, such as that induced by stress or sympathomimetic agents including caffeine, may include symptoms such as elevated motility and altered absorption. Modulation of gastrointestinal physiology is taught for applications including but not limited to the maintenance of baseline levels of gastrointestinal motility, secretion, absorption, and hormone release. Modulation of gastrointestinal physiology is also taught for applications including but not limited to the real-time control of levels of gastrointestinal motility, secretion, absorption, and hormone release, in response to physiological needs as well as in response to perturbations. Such external perturbation that can induce symptoms that are alleviated by the present invention include but are not limited to stress, consumption of caffeine, alcohol, or other substance, consumption of allergenic substance, or consumption of infectious or toxic agent. By intervening with the application of autonomic modulation to counter these undesirable autonomic responses to external agents, these side effects are reduced or prevented.

F. Immunomodulation. An object of the present invention, as taught in the parent case, is the modulation of the autonomic nervous system for physiologic modulation, including modulation of immune system physiology. By altering the level of sympathetic nervous system activity, or the level of parasympathetic nervous system activity, or the ratio of sympathetic to parasympathetic nervous system activity (as reflected in metrics such as the autonomic index), the level of activity of the immune system may be modulated. Both polarities of modulation have efficacy in the treatment of disease as well as in prophylactic applications.

Modulation, including up-regulation of the immune system, through autonomic modulation, as taught in the parent case invention has application to the treatment of infection, cancer, autoimmune immunodeficiency syndrome (AIDS), human immunodeficiency virus) infection (HIV), severe combined immunodeficiency (SCID), other causes of immunodeficiency, other causes of immunosuppression, mitigation of effects of iatrogenic immunosuppression (including that used with organ transplantation or for treating autoimmune disorders), and other causes of decreased immune system activity.

Modulation, including down-regulation, of the immune system, through autonomic modulation, as taught in the parent case invention has application to the treatment of autoimmune disease, including but not limited to multiple sclerosis, reflex sympathetic dystrophy (RSD), type I diabetes (the pathophysiology of which may include an autoimmune component), rheumatoid arthritis, graft versus host disease, psoriasis, allergic reactions, dermatitis, other allergic conditions, other diseases involving signs or symptoms due to an autoimmune or other immune pathology, and other diseases with untoward effects arising from excessive or detrimental immune responses.

Modulation, including down-regulation, of the immune system, through autonomic modulation, as taught in the parent case invention has application to the treatment of some complications from infection, including but not limited to Lyme disease, streptococcal pharyngitis (strep throat), rheumatic heart disease, fungal infections, parasitic infections, bacterial infections, viral infections, other infections, and other exposures to infectious or allergenic agents.

Modulation, including down-regulation, of the immune system, through autonomic modulation, as taught in the parent case invention has application to the augmentation of other therapies, and may be used to suppress immune function in patients with organ transplantation.

G. Asthma. An object of the present invention, as taught in the parent case, is the modulation of the autonomic nervous system for physiologic modulation, including modulation of pulmonary physiology. By altering the level of sympathetic nervous system activity, or the level of parasympathetic nervous system activity, or the ratio of sympathetic to parasympathetic nervous system activity (as reflected in metrics such as the autonomic index), the level of activity of the immune system may be modulated. Both polarities of modulation have efficacy in the treatment of disease as well as in prophylactic applications.

Modulation, including stimulation of the sympathetic nervous system, as taught in the parent case invention has application to the treatment of asthma, including exercise induced asthma and other forms of asthma. Through stimulation of the sympathetic nervous system, the beta-2 efferent pathways of the sympathetic nervous system are activated, effecting bronchodilation, providing a therapeutic action opposing the bronchoconstrictive process that underlies the increased airway resistance which results in the potentially life-threatening signs and symptoms of this disease. This same therapy is also applied to the treatment of bronchospasm and laryngospasm, in which elevated sympathetic efferent activity mitigates the constrictive effects on the airway.

Modulation, including stimulation of the sympathetic nervous system and stimulation of the parasympathetic nervous system, as taught in the parent case invention has application to the treatment of asthma, including exercise induced asthma through an additional mechanism. Through inhibition of the sympathetic nervous system, the activity of the immune system may be down-regulated, reducing the sensitivity of the pulmonary mast cells to allergens, thereby reducing the susceptibility to and the severity of asthma signs and symptoms.

H. Cardiovascular Disease—Cardiac. An object of the present invention, as taught in the parent case, is the modulation of the autonomic nervous system for physiologic modulation, including modulation of cardiovascular physiology, including cardiac physiology in particular. By altering the level of sympathetic nervous system activity, or the level of parasympathetic nervous system activity, or the ratio of sympathetic to parasympathetic nervous system activity (as reflected in metrics such as the autonomic index), cardiac parameters may be modulated. Both polarities of modulation have efficacy in the treatment of cardiac disease as well as in prophylactic applications.

Modulation, including stimulation of the sympathetic nervous system, inhibition of the parasympathetic system, or increase in the autonomic index, as taught in the parent case invention has application to the treatment of cardiac disease, including hear failure and bradycardia. Through stimulation of the sympathetic nervous system, the beta-1 efferent pathways of the sympathetic nervous system are activated, effecting increase inotropic activity, providing a therapeutic action to mitigate decreased myocardial contractility found in cardiac disease, including congestive heart failure, post myocardial infarction sequelae, and other cardiac disorders. Sympathetic stimulation is also used to effect increased chronotropic behavior, thereby elevating heart rate. This has application to numerous cardiac conditions, including bradycardia and heart block. This has further application to the treatment of hypotension and to neurogenic shock, which may be augmented by autonomic neuromodulation directed toward the vascular system, as described below.

Modulation, including inhibition of the sympathetic nervous system, stimulation of the parasympathetic system, or decrease in the autonomic index, as taught in the parent case invention has application to the treatment of cardiac disease. The negative inotropic effect of such autonomic modulation has application to cardiac disease, including among others, diastolic disease, in which the heart muscle does not fully relax, thereby impairing proper atrial and ventricular filling during the diastolic portion of the cardiac cycle. This additionally has application to the treatment of hypertension, through each of negative inotropic and negative chronotropic effects. This further has application to the prevention and control of the progression of congestive heart failure, through the reduction of the normal sympathetic physiologic response to heart failure, which itself contributes to progression of the disease. The negative chronotropic effect of such modulation also has application to the treatment of tachycardia and other cardiac rhythm abnormalities.

I. Cardiovascular Disease—Vascular. An object of the present invention, as taught in the parent case, is the modulation of the autonomic nervous system for physiologic modulation, including modulation of cardiovascular physiology including vascular physiology in particular. By altering the level of sympathetic nervous system activity, or the level of parasympathetic nervous system activity, or the ratio of sympathetic to parasympathetic nervous system activity (as reflected in metrics such as the autonomic index), the level of activity including the muscular tone of the vascular system may be modulated. Both polarities of modulation have efficacy in the treatment of disease as well as in prophylactic applications.

Modulation, including stimulation of the sympathetic nervous system, inhibition of the parasympathetic nervous system, or increase in the autonomic index, as taught in the parent case invention has application to the treatment of hypotension and neurogenic shock, and other conditions in which vascular tone or blood pressure is below normal. This further has application to therapeutically increase vascular tone or blood pressure, including to levels above normal, such as in the treatment of cerebral vasospasm, ischemic stroke, peripheral vascular disease, or other condition. Through stimulation of the sympathetic nervous system, the alpha-1 efferent pathways of the sympathetic nervous system are activated, effecting vasoconstriction, providing a therapeutic action to correct low blood pressure as well as to provide a normalizing to correct low vascular tone characterizing neurogenic shock as well as to elevate blood pressure to treat the above listed conditions. A particular advantage of this therapy is conveyed by the ability to selectively rather than systemically induce vasoconstriction, thereby elevating systemic blood pressure while avoiding vasoconstriction in selected circulatory regions, as desired in the treatment of cerebral vasospasm.

Modulation, including inhibition of the sympathetic nervous system, stimulation of the parasympathetic nervous system, or decrease in the autonomic index, as taught in the parent case invention has application to the treatment of hypertension, including essential hypertension, renally mediated hypertension, atherosclerosis mediated hypertension, other forms of systemic hypertension, and pulmonary hypertension. Through this therapy, vasodilation is achieved, which is also used to treat coronary artery disease, peripheral vascular disease, cerebral vascular disease, myocardial infarction, and stroke. This has further use in other therapy in which enhanced circulation is desired, such as for enhanced circulation and drug delivery in the treatment of infections and as an adjuvant to accelerate healing processes, such as ulcers, postoperative wounds, trauma, and other conditions.

J. Headaches. An object of the present invention, as taught in the parent case, is the modulation of the autonomic nervous system for physiologic modulation, including modulation of cerebral vascular physiology, including intraparenchymal circulation and meningeal circulation. By altering the level of sympathetic nervous system activity, or the level of parasympathetic nervous system activity, or the ratio of sympathetic to parasympathetic nervous system activity (as reflected in metrics such as the autonomic index), the level of activity of the cerebral vascular system may be modulated. Both polarities of modulation have efficacy in the treatment of headaches as well as in prophylactic applications.

Modulation, including stimulation of the sympathetic nervous system, inhibition of the parasympathetic nervous system, or increase in the autonomic index, as taught in the parent case invention has application to the treatment of headaches, including migraine headaches, cluster headaches, and other headaches. Through stimulation of the sympathetic nervous system, the alpha-1 efferent pathways of the sympathetic nervous system are activated, effecting cerebral vasoconstriction, providing decrease in the blood volume within the intracranial vascular structures as well as the remainder of the intracranial compartment. This acts through additional mechanisms including but not limited to reduction of the mechanical tension on the dura, reduction of the intracranial pressure, and alteration in the blood flow and neural activity within the brain, altering neural and vascular patterns that can progress to generate headaches or other undesirable neural states.

Modulation, including inhibition of the sympathetic nervous system, stimulation of the parasympathetic nervous system, or decrease in the autonomic index, as taught in the parent case invention has application to the prophylaxis and treatment of headaches, including migraine headaches, cluster headaches, and other headaches. Through inhibition of the sympathetic nervous system, the activity of alpha-1 efferent pathways of the sympathetic nervous system are reduced, effecting cerebral vasodilation, providing variation in the vascular tone as well as altered blood flow and neural activity, which has application to disrupt neural and vascular patterns that can generate headaches or other undesirable neural states.

K. Smoking Cessation and Drug Withdrawal. An object of the present invention, as taught in the parent case, is the modulation of the autonomic nervous system, which has application to stabilize or oppose the physiologic response to the introduction or withdrawal of pharmacological or other bioactive agents, including nicotine, caffeine, stimulants, depressants, and other medical and recreational drugs.

When patients cease smoking, the nicotine plasma levels drop, reducing the level of stimulation of the nicotinic receptors in the sympathetic nervous system. This alteration causes a physiologic response characterized by significant levels of anxiety and a withdrawal response in the person. By modulating the sympathetic nervous system activity using the method and apparatus taught in the parent case or using variants thereof, this response can be mitigated. This has application to controlling addiction to nicotine and in the facilitation of smoking cessation.

When patients cease intake of alcohol, narcotics, sedatives, hypnotics, or other drugs to which they may be addicted, a withdrawal response ensues. This response can be life threatening. In alcohol withdrawal, delirium tremens can be accompanied by dangerous elevations in heart rate. By modulating sympathetic and/or parasympathetic activity to control the autonomic index, this response can be reduced or prevented.

L. Hyperhidrosis. An object of the present invention, as taught in the parent case, is the modulation of the autonomic nervous system, which has application to prevent or control the symptoms of hyperhidrosis.

In hyperhidrosis, an abnormally active or responsive sympathetic nervous system results is excessive perspiration, typically most problematic when involving the hands and axillae. Current treatments employ surgical ablation of the corresponding region of the sympathetic trunk, which results in irreversible cessation of sympathetic activity in the corresponding anatomical region. By modulating the sympathetic nervous system activity using the method and apparatus taught in the parent case or using variants thereof, the symptoms arising from this condition can be prevented or reduced.

M. Reflex Sympathetic Dystrophy and Pain. An object of the present invention, as taught in the parent case, is the modulation of the autonomic nervous system, which has application to prevent the development or progression of reflex sympathetic dystrophy and to control the symptoms once the condition has developed.

Reflex sympathetic dystrophy is a potentially debilitating condition that typically develops following trauma to a peripheral nerve, in which a crush or transection injury disrupts the afferent pain fibers and the sympathetic efferent fibers. The most widely accepted theory as to the etiology underlying this condition holds that during the healing phase, sympathetic efferent fibers develop connections with the pain carrying afferent fibbers, resulting in the perception of pain in response to sympathetic activity. Current therapy involves pharmacologic agents and is largely ineffective, leaving a population of otherwise often healthy people who are debilitated by severe chronic medication refractory pain. By modulating the sympathetic nervous system activity using the method and apparatus taught in the parent case or using variants thereof, the symptoms arising from reflex sympathetic dystrophy can be prevented or reduced.

Inhibition of sympathetic system activity is used to reduce the level of neural activity that is pathologically fed back into pain afferent fibers, thereby reducing symptoms. This therapy may be applied preventatively to modulate sympathetic nervous system activity and minimize the degree of neural connection between the sympathetic efferent neurons and the pain carrying afferent neurons.

N. General—Control and Temporal Modulation. Various forms of temporal modulation may be performed to achieve the desired efficacy in the treatment of these and other diseases, conditions, or augmentation applications. Constant intensity modulation, time varying modulation, cyclical modulation, altering polarity modulation, up-regulation interspersed with down-regulation, intermittent modulation, and other permutations are include in the present invention. The use of a single or multiplicity of these temporal profiles provides resistance of the treatment or enhancement to habituation by the nervous system, thereby preserving or prolonging the effect of the modulation. The use of a multiplicity of modulation sites provides resistance of the treatment or enhancement to habituation by the nervous system, thereby preserving or prolonging the effect of the modulation; by distributing or varying the intensity of the neuromodulation among a plurality of sites enables the delivery of therapy or augmentation that is more resistant to adaptation or habituation by the nervous system. Furthermore, the control of neural state, including level of sympathetic nervous system activity, level of parasympathetic nervous system activity, autonomic index, or other characteristic or metric of neural function in either or both of an open-loop or closed-loop manner is taught herein. The use of open-loop or closed-loop control to maintain at least one neural state at a constant or time varying target level is used to better control physiology, reduce habituation, reduce side effects, apportion side effect to preferable time windows such as while sleeping), and optimize response to therapy.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

Furthermore, all patents claiming priority to U.S. Provisional Application 60/095,413 and U.S. application Ser. No. 09/340,326, now U.S. Pat. No. 6,366,813, which were filed prior to Dec. 15, 2004 or which are in content continuations of these are incorporated by reference. Specifically, this includes U.S. Provisional Application 60/095,413 filed Aug. 5, 1998, U.S. Utility application Ser. No. 09/340,326 filed Jun. 25, 1999 and now U.S. Pat. No. 6,366,813, U.S. Utility application Ser. No. 10/008,576 filed Nov. 11, 2001 and now U.S. Pat. No. 6,819,956, U.S. Provisional Application 60/427,699 filed Nov. 20, 2002, U.S. Provisional Application 60/436,792 filed Dec. 27, 2002, U.S. Utility application Ser. No. 10/718,248 filed Nov. 20, 2003, U.S. Provisional Application 60/438,286 filed Jan. 6, 2003, U.S. Utility application Ser. No. 10/753,205 filed Jan. 6, 2004, U.S. Provisional Application 60/460,140 filed Apr. 3, 2003, U.S. Utility application Ser. No. 10/818,333 filed Apr. 5, 2004, U.S. Provisional Application 60/562,487 filed Apr. 14, 2004, U.S. application Ser. No. 10/889,844 filed Jul. 12, 2004, U.S. Application 60/614,241 filed Sep. 28, 2004, U.S. Provisional Application 60/307,124 Jul. 23, 2001, U.S. application Ser. No. 10/198,871 filed Jul. 19, 2002, and other which may not have published yet.

### BRIEF DESCRIPTION OF DRAWINGS

FIG. 1 depicts GastroPace™ implanted along the Superior Greater Curvature of the stomach for both Neural Afferent and Neuromuscular Modulation.

FIG. 2 depicts GastroPace™ implanted along the Inferior Greater Curvature of the stomach for both Neural Afferent and Neuromuscular Modulation.

FIG. 3 depicts GastroPace™ implanted along the Pyloric Antrum of the stomach for both Neural Afferent and Neuromuscular Modulation.

FIG. 4 depicts GastroPace™ implanted adjacent to the Gastric Pylorus for modulation of pylorus activity and consequent control of gastric food efflux and intraluminal pressure.

FIG. 5 depicts GastroPace™ implanted along the Pyloric Antrum of the stomach with modulators positioned for stimulation of Neural and Neuromuscular structures of the Pylorus and Pyloric Antrum of the Stomach.

FIG. 6 depicts GastroPace™ implanted along the Pyloric Antrum of the stomach with modulators positioned for stimulation of Neural and Neuromuscular structures of the Pylorus, Pyloric Antrum, Greater Curvature, and Lesser Curvature of the Stomach.

FIG. 7 depicts the Nerve Cuff Electrode, comprising the Epineural Electrode Nerve Cuff Design.

FIG. 8 depicts the Nerve Cuff Electrode, comprising the Axial Electrode Blind End Port Design.

FIG. 9 depicts the Nerve Cuff Electrode, comprising the Axial Electrode Regeneration Port Design.

FIG. 10 depicts the Nerve Cuff Electrode, comprising the Axial Regeneration Tube Design.

FIG. 11 depicts GastroPace™ implanted along the Pyloric Antrum of the stomach with modulators positioned for stimulation of Afferent Neural Structures, including sympathetic and parasympathetic fibers.

FIG. 12 depicts GastroPace™ implanted along the Pyloric Antrum of the stomach with modulators positioned for stimulation of Neural and Neuromuscular structures of the Pylorus, Pyloric Antrum, Greater Curvature, and Lesser Curvature of the Stomach and with modulators positioned for stimulation of Afferent Neural Structures, including sympathetic and parasympathetic fibers.

FIG. 13 depicts the Normal Thoracoabdominal anatomy as seen via a sagittal view of an open dissection.

FIG. 14 depicts modulators for GastroPace™ positioned on the sympathetic trunk and on the greater and lesser splanchnic nerves, both supradiaphragmatically and infradiaphragmatically, for afferent and efferent neural modulation.

FIG. 15 depicts GastroPace™ configured with multiple pulse generators, their connecting cables, and multiple modulators positioned on the sympathetic trunk and on the greater and lesser splanchnic nerves, both supradiaphragmatically and infradiaphragmatically, for afferent and efferent neural modulation.

FIG. 16 depicts GastroPace™ configured with multiple pulse generators, their connecting cables, and multiple modulators positioned on the sympathetic trunk and on the greater and lesser splanchnic nerves, both supradiaphragmatically and infradiaphragmatically, for afferent and efferent neural modulation and with modulators positioned for stimulation of Neural and Neuromuscular structures of the Pylorus, Pyloric Antrum, Greater Curvature, and Lesser Curvature of the Stomach.

FIG. 17 depicts the Normal Spinal Cord Anatomy, shown in Transverse Section.

FIG. 18 depicts GastroPace™ implanted with multiple modulators positioned for modulation of Spinal Cord structures

FIG. 19 depicts the three muscle layers of the stomach.

FIG. 20 depicts GastroPace™ with modulators implanted along the surface of the stomach.

FIG. 21 depicts GastroPace™ with an array of modulators implanted along the surface of the stomach.

FIG. 22 depicts a GastroPace™ array, with multiple pulse generators implanted. This figure is exemplary, with two pulse generators shown each in the thorax and abdomen, each connected to modulators.

FIG. 23 depicts GastroPace™, with two pulse generators shown in an exemplary configuration in the abdomen, each connected to modulators.

FIG. 24 depicts GastroPace™, in a close up view of modulators implanted in the abdomen.

FIG. 25 depicts GastroPace™, in a close up view of modulators implanted in the abdomen.

FIG. 26 depicts GastroPace™, in a close up view of modulators and modulator arrays implanted in the abdomen.

FIG. 27 depicts GastroPace™, in a close up view of the modulators implanted adjacent to the spinal cord, spinal nerves, dorsal root ganglia, and adjacent structures.

FIG. 28 depicts GastroPace™, in a detailed view of that shown in the parent case in FIG. 15, with more detail of the modulators shown. This figure shows exemplary modulators of the design shown in FIG. 7.

FIG. 29 depicts GastroPace™, in a detailed view of that shown in the parent case in FIG. 15, with more detail of the modulators shown. This figure shows exemplary modulators similar to the catheter design shown in FIG. 35.

FIG. 30 depicts GastroPace™, in a detailed view of that shown in the parent case in FIG. 15, with more detail of the modulators shown. This figure shows exemplary modulators a wireless catheter design.

FIG. 31 depicts GastroPace™, in a detailed view of that shown in the parent case in FIG. 15, with more detail of the modulators shown. This figure shows exemplary modulators a wireless cylindrical or injectable implant design.

FIG. 32 depicts GastroPace™, in a detailed view of that shown in the parent case in FIG. 15, with more detail of the modulators shown. This figure shows exemplary modulators similar to the catheter design shown in FIG. 35.

FIG. 33 depicts electrode catheter being implanted with surgical tools.

FIG. 34 depicts electrode catheter being implanted with surgical tools.

FIG. 35 depicts neuromodulatory interface array catheter in detailed view.

FIG. 36 depicts neurophysiological effects of GastroPace™ functions, with view of time course of response of autonomic index to modulation of at least one of sympathetic and parasympathetic nervous systems.

FIG. 37 is a schematic diagram of one embodiment of the present invention implanted in a human patient.

FIG. 38 is an architectural block diagram of one embodiment of the neurological control system of the present invention.

FIG. 39 is a schematic diagram of electrical stimulation waveforms for neural modulation.

FIG. 40 is a schematic diagram of one example of the recorded waveforms.

FIG. 41 is a diagram that depicts metabolic modulation.

FIG. 42 is a diagram depicting satiety modulation or appetite modulation.

FIGS. 43, 44, and 45 show preclinical animal data from an experiment designed to characterize efficacy of the invention taught in the parent and subsequent patent applications.

FIG. 46 depicts a detailed view of a wireless implementation of the invention taught in the parent case and subsequent cases case in FIG. 15, with detail of the wireless neuromodulators.

FIG. 47 depicts instruments and other apparatus in use during a procedure in which neuromodulators are being implanted in regions including but not limited to the epidural or subdural spaces.

FIG. 48 depicts instruments and other apparatus in use during a procedure in which neuromodulators are being implanted in regions including but not limited to the epidural or subdural spaces.

FIG. 49 depicts instruments and other apparatus in use during a procedure in which neuromodulators are being implanted in regions including but not limited to the epidural or subdural spaces.

FIG. 50 depicts neuromodulators, instruments, and other apparatus in use during a procedure in which neuromodulators are being implanted in regions including but not limited to at least one of the prevertebral, paravertebral, retroperitoneal, intraperitoneal, retropleural, intrapleural, cervical, thoracic, abdominal, and pelvic spaces.

FIG. 51 is a system block diagram of Neuromodulatory System (NMS).

FIG. 52 is a table listing various biomarkers that may be used in conjunction with the present invention

FIG. 53 depicts neuromodulators, instruments, and other apparatus including the in use during a procedure in which minimally invasive neuromodulators are being implanted in regions including but not limited to at least one of the prevertebral, paravertebral, retroperitoneal, intraperitoneal, retropleural, intrapleural, cervical, thoracic, abdominal, and pelvic spaces.

FIG. A02 depicts biochemical pathways involved in mechanism of action of the effect of sympathetic nervous system modulation on adipose tissue and lipolysis.

FIG. 54 depicts implanted or nonimplanted neuromodulators configured to be in communication with regions including but not limited to at least one of the prevertebral, paravertebral, retroperitoneal, intraperitoneal, retropleural, intrapleural, cervical, thoracic, abdominal, and pelvic spaces.

FIG. 55 depicts implanted or nonimplanted neuromodulators configured to be in communication with regions including but not limited to at least one of the prevertebral, paravertebral, retroperitoneal, intraperitoneal, retropleural, intrapleural, cervical, thoracic, abdominal, and pelvic spaces.

FIG. 56 depicts one preferred or exemplary configuration of nonimplanted or implanted neuromodulators comprising Autonomic Vector control System (AVCS) in communication with neural or other target structures.

FIG. 57 depicts one preferred or exemplary configuration of nonimplanted or implanted neuromodulators comprising Autonomic Vector control System (AVCS) in communication with neural or other target structures.

### DETAILED DESCRIPTION OF THE INVENTION

The present invention encompasses a multimodality technique, method, and apparatus for the treatment of several diseases, including but not limited to obesity, eating disorders, depression, epilepsy, and diabetes.

These modalities may be used for diagnostic and therapeutic uses, and these modalities include but are not limited to stimulation of gastric tissue, stimulation of gastric musculature, stimulation of gastric neural tissue, stimulation of sympathetic nervous tissue, stimulation of parasympathetic nervous tissue, stimulation of peripheral nervous tissue, stimulation of central nervous tissue, stimulation of cranial nervous tissue, stimulation of skin receptors, including Pacinian corpuscles, nociceptors, Golgi tendons, and other sensory tissues in the skin, subcutaneous tissue, muscles, and joints.

Stimulation may be accomplished by electrical means, optical means, electromagnetic means, radiofrequency means, electrostatic means, magnetic means, vibrotactile means, pressure means, pharmacologic means, chemical means, electrolytic concentration means, thermal means, or other means for altering tissue activity.

Already encompassed in the above description are several specific applications of this broad technology. These specific applications include electrical stimulation of gastric tissue, including at least one of muscle and neural, for the control of appetite and satiety, and for the treatment of obesity. Additional specific uses include electrical stimulation of gastric tissue for the treatment of depression. Further uses include electrical stimulation of pancreatic tissue for the treatment of diabetes.

A. Satiety Modulation.

A1. Sympathetic Afferent Stimulation. Selected stimulation of the sympathetic nervous system is an objective of the present invention. A variety of modulator designs and configurations are included in the present invention and other designs and configurations may be apparent to those skilled in the art and these are also included in the present invention. Said modulator may take the form of electrode or electrical source, optical source, electromagnetic source, radiofrequency source, electrostatic source, magnetic source, vibrotactile source, pressure source, pharmacologic source, chemical source, electrolyte source, thermal source, or other energy or stimulus source.

One objective of the modulator design for selective sympathetic nervous system stimulation is the avoidance of stimulation of the vagus nerve. Stimulation of the vagus nerve poses the risk enhanced propensity for development of gastric or duodenal ulcers.

Other techniques in which electrical stimulation has been used for the treatment of obesity have included stimulation of central nervous system structures or peripheral nervous system structures. Other techniques have used sequential stimulation of the gastric tissue to interrupt peristalsis; however, this broad stimulation of gastric tissue necessarily overlaps regions heavily innervated by the vagus nerve and consequently poses the same risks of gastric and duodenal ulcers that stimulation of the vagus nerve does.

One objective of the present invention is the selective stimulation of said afferent neural fibers that innervate gastric tissue. Avoidance of vagus nerve stimulation is an object of this modulator configuration. Other alternative approaches to gastric pacing involving gastric muscle stimulation secondarily cause stimulation of the vagus nerve as well as stimulation of gastric tissues in acid-secreting regions, consequently posing the undesirable side effects of gastric and duodenal ulcers secondary to activation of gastric acid stimulation.

There are a number of approaches to selective stimulation of the sympathetic nervous system. This invention includes stimulation of the sympathetic fibers at sites including the zones of innervation of the stomach, the gastric innervation zones excluding those innervated by vagus branches, the distal sympathetic branches proximal to the stomach, the sympathetic trunk, the intermediolateral nucleus, the locus ceruleus, the hypothalamus, and other structures comprising or influencing sympathetic afferent activity.

Stimulation of the sympathetic afferent fibers elicits the perception of satiety, and achievement of chronic, safe, and efficacious modulation of sympathetic afferents is one of the major objectives of the present invention.

Alternating and augmenting stimulation of the sympathetic nervous system and vagus nerve is included in the present invention. By alternating stimulation of the vagus nerve and the sympathetic afferent fibers, one may induce the sensation of satiety in the implanted patient while minimizing the potential risk for gastric and duodenal ulcers.

Since vagus and sympathetic afferent fibers carry information that is related to gastric distention, a major objective of the present invention is the optimization stimulation of the biggest fibers, the afferent sympathetic nervous system fibers, and other afferent pathways such that a maximal sensation of satiety is perceived in the implanted individual and such that habituation of this sensation of satiety is minimized. This optimization is performed in any combination of matters including temporal patterning of the individual signals to each neural pathway, including but not limited to the vagus nerve and sympathetic afferents, as well as temporal patterning between a multiplicity of stimulation channels involving the same were neural pathways. The present invention teaches a multiplicity of apparatus and method for stimulation of afferent sympathetic fibers, as detailed below. Other techniques and apparatus may become apparent to those skilled in the art, without departing from the present invention.

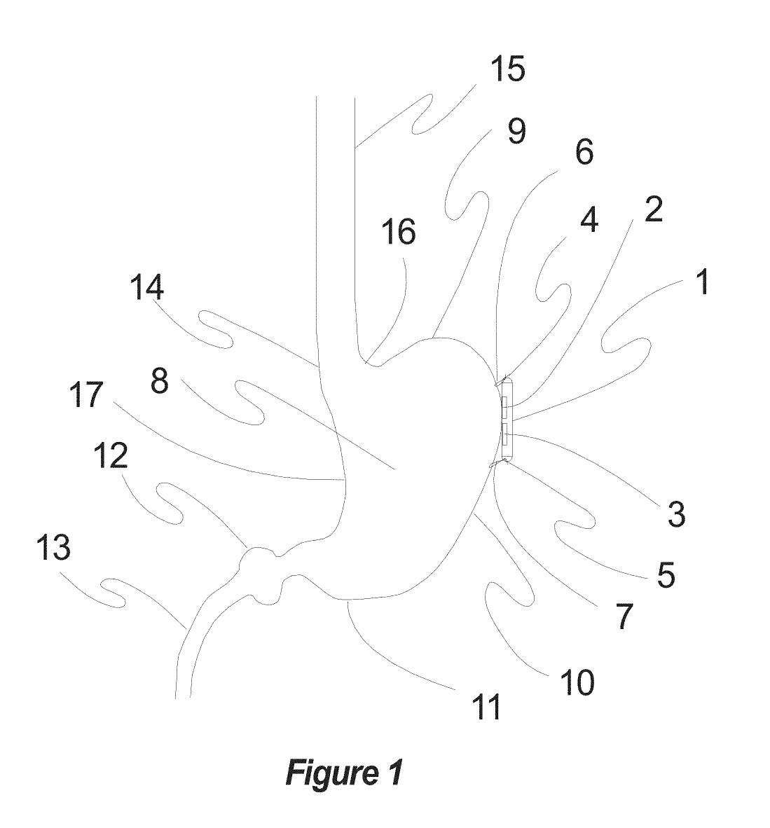

Ala. Sympathetic Afferents—Gastric Region. FIG. 1 through FIG. 3 demonstrate stimulation of gastric tissue, including at least one of neural and muscular tissue. Anatomical structures include esophagus 15, lower esophageal sphincter 14, stomach 8, cardiac notch of stomach 16, gastric fundus 9, greater curvature of stomach 10, pyloric antrum 11, lesser curvature of stomach 17, pylorus 12, and duodenum 13.

Implantable pulse generator 1 is shown with modulator 2 and modulator 3 in contact with the corresponding portion of stomach 8 in the respective figures, detailed below. Implantable pulse generator further comprises attachment fixture 4 and attachment fixture 5. Additional or fewer attachment fixtures may be included without departing from the present invention. Attachment means 6 and attachment means 7 are used to secure attachment fixture 4 and attachment fixture 5, respectively to appropriate portion of stomach 8. Attachment means 6 and attachment means 7 may be comprised from surgical suture material, surgical staples, adhesives, or other means without departing from the present invention.

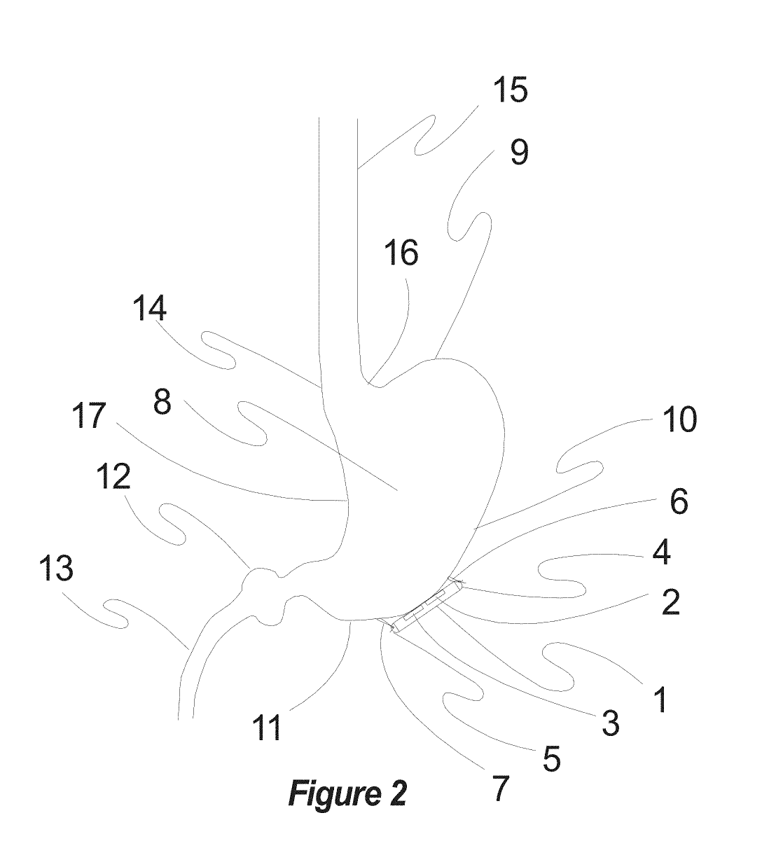

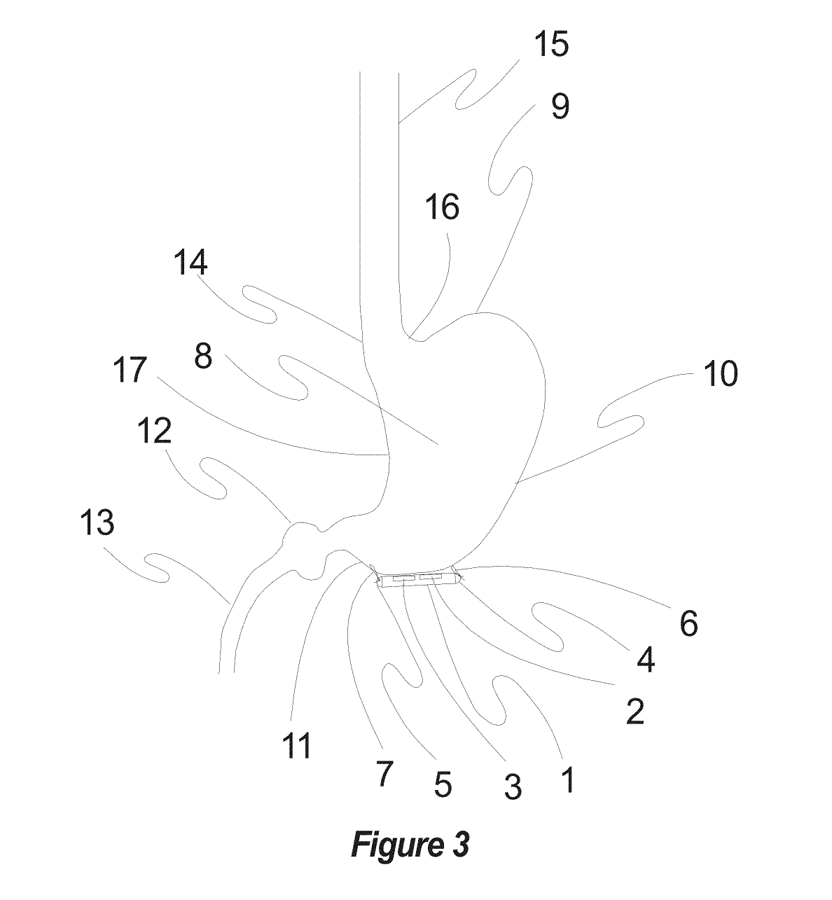

FIGS. 1,2, and 3 show implantable pulse generator 1 in several anatomical positions. In FIG. 1, implantable pulse generator 1 is shown positioned along the superior region of the greater curvature of stomach —10, with modulator 2 and modulator 3 in contact with the tissues comprising the greater curvature of stomach 10. In FIG. 2, implantable pulse generator 1 is shown positioned along the inferior region of the greater curvature of stomach 10, with modulator 2 and modulator 3 in contact with the tissues comprising the greater curvature of stomach 10. In FIG. 3, implantable pulse generator 1 is shown positioned along the pyloric antrum 11, with modulator 2 and modulator 3 in contact with the tissues comprising the pyloric antrum 11.

Modulator 2 and modulator 3 are used to stimulate at least one of gastric longitudinal muscle layer, gastric circular muscle layer, gastric nervous tissue, or other tissue. Modulator 2 and modulator 3 may be fabricated from nonpenetrating material or from penetrating material, including needle tips, arrays of needle tips, wires, conductive sutures, other conductive material, or other material, without departing from the present invention.

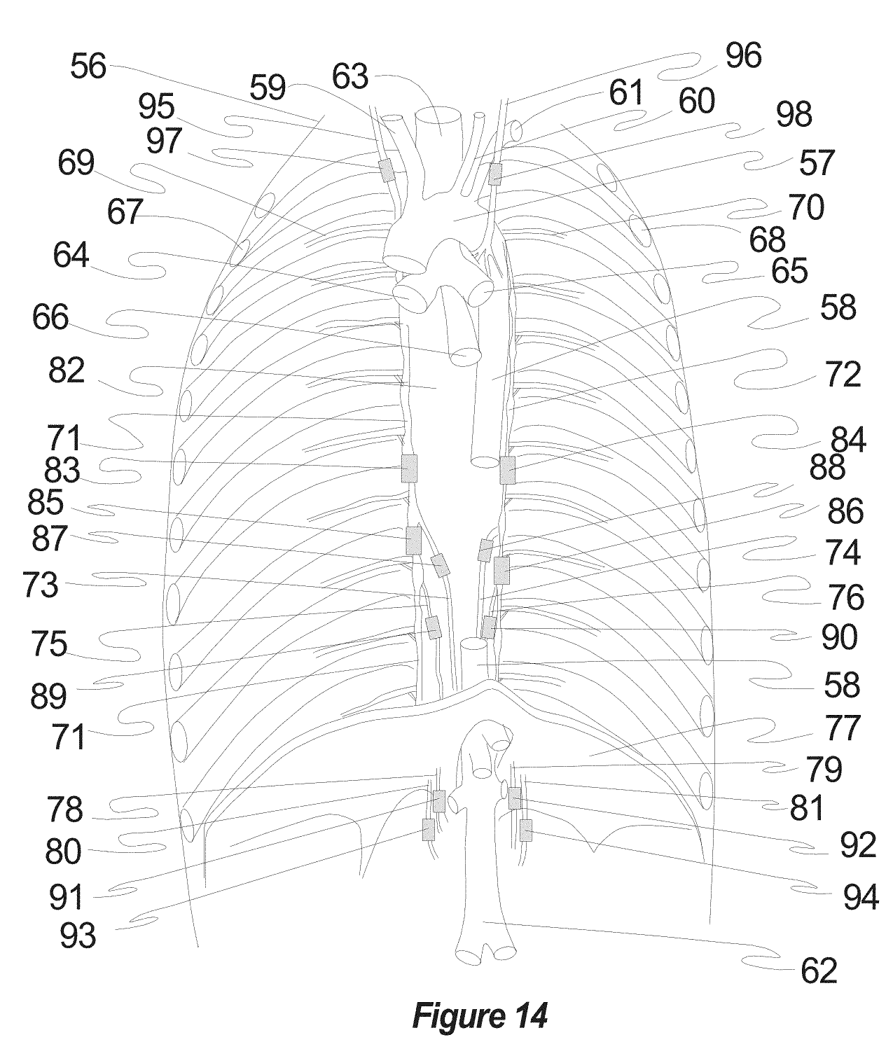

Alb. Sympathetic Afferents—Sympathetic Trunk. The present invention teaches apparatus and method for stimulation of sympathetic afferent fibers using stimulation in the region of the sympathetic trunk. As shown in FIGS. 14, 15, and 16, sympathetic trunk neuromodulatory interface 83 and 85, positioned on right sympathetic trunk 71, and sympathetic trunk neuromodulatory interface 85, 86 positioned on left sympathetic trunk —72, are used to provide stimulation for afferent as well as for efferent sympathetic nervous system modulation. Modulation of efferent sympathetic nervous system is discussed below, and this is used for metabolic modulation.

Alc. Sympathetic Afferents—Other. The present invention teaches apparatus and method for stimulation of sympathetic afferent fibers using stimulation of nerves arising from the sympathetic trunk. As shown in FIGS. 14, 15, and 16, thoracic splanchnic neuromodulatory interface 87, 89, 88, and 90, positioned on right greater splanchnic nerve 73, right lesser splanchnic nerve 75, left greater splanchnic nerve 74, left lesser splanchnic nerve 76, respectively, and are used to provide stimulation for afferent as well as for efferent sympathetic nervous system modulation. Modulation of efferent sympathetic nervous system is discussed below, and this is used for metabolic modulation.

A2. Gastric Musculature Stimulation. A further object of the present invention is the stimulation of the gastric musculature. This may be performed using either or both of closed loop and open loop control. In the present embodiment, a combination of open and closed loop control is employed. The open loop control provides a baseline level of gastric stimulation. This stimulation maintains tone of the gastric musculature. This increases the wall tension the stomach and plays a role in the perception of satiety in the implanted patient. Additionally, stimulation of the gastric musculature causes contraction of the structures, thereby reducing the volume of the stomach. This gastric muscle contraction, and the consequent reduction of stomach volume effectively restricts the amount of food that may be ingested. Surgical techniques have been developed and are known to those practicing in the field of surgical treatment of obesity. Several of these procedures are of the restrictive type, but because of their surgical nature they are fixed in magnitude and difficult if not impossible to reverse. The present invention teaches a technique which employs neural modulation and gastric muscle stimulation which by its nature is the variable and reversible. This offers the advantages postoperative adjustment of magnitude, fine tuning for the individual patient, varying of magnitude to suit the patient's changing needs and changing anatomy over time, and the potential for reversal or termination of treatment. Furthermore, since the gastric wall tension is generated in a physiological manner by the muscle itself, it does not have the substantial risk of gastric wall necrosis and rupture inherent in externally applied pressure, as is the case with gastric banding.

FIGS. 1, 2, and 3 depict placements of the implantable pulse generator 1 that may be used to stimulate gastric muscle tissue. Stimulation of both longitudinal and circular muscle layers is included in the present invention. Stimulation of gastric circular muscle layer causes circumferential contraction of the stomach, and stimulation of gastric longitudinal muscle layer causes longitudinal contraction of the stomach.

This muscle stimulation and contraction accomplishes several objectives: (1) functional reduction in stomach volume, (2) increase in stomach wall tension, (3) reduction in rate of food bolus flow. All of these effects are performed to induce the sensation of satiety.

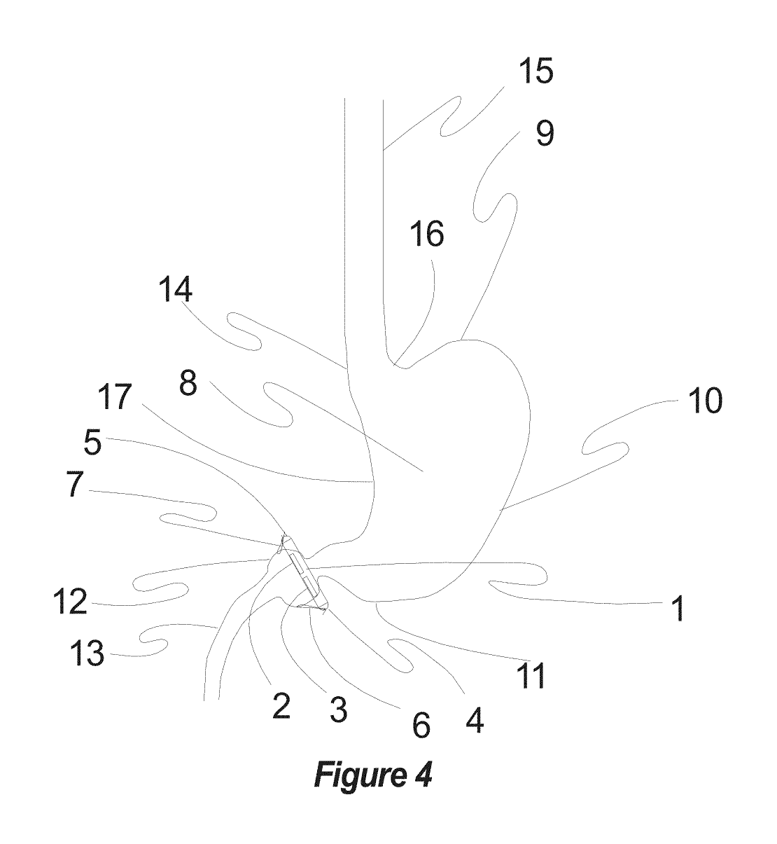

A3. Gastric Pylorus Stimulation. FIG. 4 depicts implantable pulse generator 1 positioned to perform stimulation of the gastric pylorus 12 to induce satiety by restricting outflow of food bolus material from the stomach 8 into the duodenum 13. Stimulation of the pylorus 12 may be continuous, intermittent, or triggered manually or by sensed event or physiological condition. FIG. 4 depicts implantable pulse generator 1 positioned adjacent to the gastric pylorus 12; this position provides secure modulator positioning while eliminating the risk of modulator and wire breakage inherent in other designs in which implantable pulse generator 1 is positioned remote from the gastric pylorus 12.

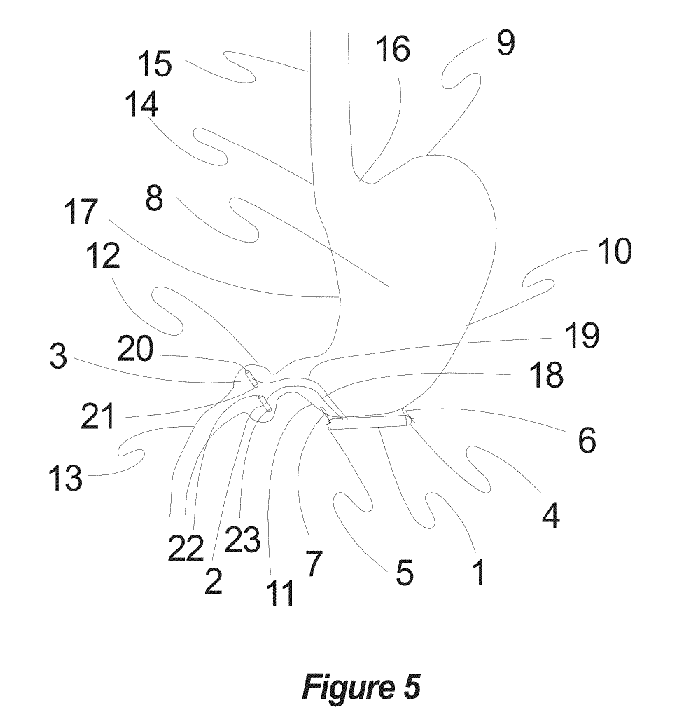

FIG. 5 depicts implantable pulse generator 1 positioned to perform stimulation of the gastric pylorus 12 to induce satiety by restricting outflow of food bolus material from the stomach 8 into the duodenum 13. Stimulation of the pylorus 12 may be continuous, intermittent, or triggered manually or by sensed event or physiological condition. FIG. 5 depicts implantable pulse generator 1 attached to stomach 8, specifically by the pyloric antrum 11; this position facilitates the use of a larger implantable pulse generator 1. The risk of modulator and wire breakage is minimized by the use of appropriate strain relief and stranded wire designs.

A4. Parasympathetic Stimulation. The parasympathetic nervous system is complementary to the sympathetic nervous system and plays a substantial role in controlling digestion and cardiac activity. Several routes are described in the present invention to modulate activity of the parasympathetic nervous system.

A4a. Parasympathetic Stimulation—Vagus Nerve. Others have advocated the use of vagus nerve stimulation for the treatment of a number of disorders including obesity. Zabara and others have described systems in which the vagus nerve in the region of the neck is stimulated. This is plagued with a host of problems, including life-threatening cardiac complications as well as difficulties with speech and discomfort during stimulation. The present invention is a substantial advance over that discussed by Zabara et al, in which unrestricted fiber activation using epineural stimulation is described. That technique results in indiscriminate stimulation of efferent and afferent fibers. With vagus nerve stimulation, efferent fiber activation generates many undesirable side effects, including gastric and duodenal ulcers, cardiac disturbances, and others.

In the present invention, as depicted in FIG. 14, vagus neuromodulatory interface 97 and 98 are implanted adjacent to and in communication with right vagus nerve 95 and left vagus nerve 96. The neuromodulatory interface 97 and 98 overcomes these limitations that have persisted for over a decade with indiscriminate vagus nerve stimulation, by selectively stimulating afferent fibers of the at least one of the vagus nerve, the sympathetic nerves, and other nerves. The present invention includes the selective stimulation of afferent fibers using a technique in which electrical stimulation is used to block anterograde propagation of action potentials along the efferent fibers. The present invention includes the selective stimulation of afferent fibers using a technique in which stimulation is performed proximal to a nerve transection and in which the viability of the afferent fibers is maintained. One such implementation involves use of at least one of neuromodulatory interface 34 which is of the form shown in at least one of Longitudinal Electrode Neuromodulatory Interface 118, Longitudinal Electrode Regeneration Port Neuromodulatory Interface 119, Regeneration Tube Neuromodulatory Interface 120, neuromodulatory interface array catheter 284 or other design which may become apparent to one skilled in the art, including designs in which a subset of the neuronal population is modulated.

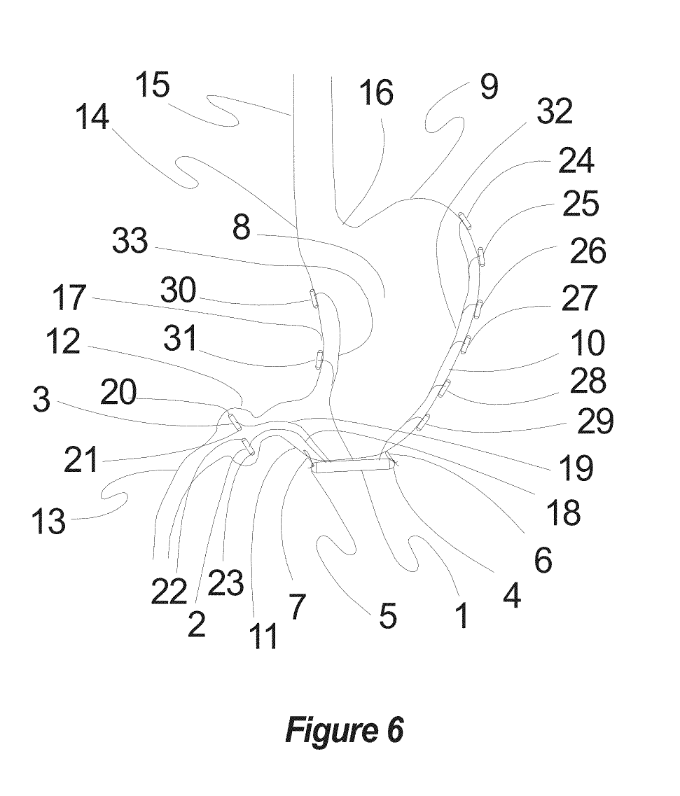

A.4.a.i. Innovative Stimulation Anatomy. FIG. 6 depicts multimodal treatment for the generation of satiety, using sympathetic stimulation, gastric muscle stimulation, gastric pylorus stimulation, and vagus nerve stimulation. This is described in more detail below. Modulators 30 and 31 are positioned in the general region of the lesser curvature of stomach 17. Stimulation in this region results in activation of vagus nerve afferent fibers. Stimulation of other regions may be performed without departing from the present invention. In this manner, selective afferent vagus nerve stimulation may be achieved, without the detrimental effects inherent in efferent vagus nerve stimulation, including cardiac rhythm disruption and induction of gastric ulcers.

A.4.a.ii. Innovative Stimulation Device. The present invention further includes devices designed specifically for the stimulation of afferent fibers.

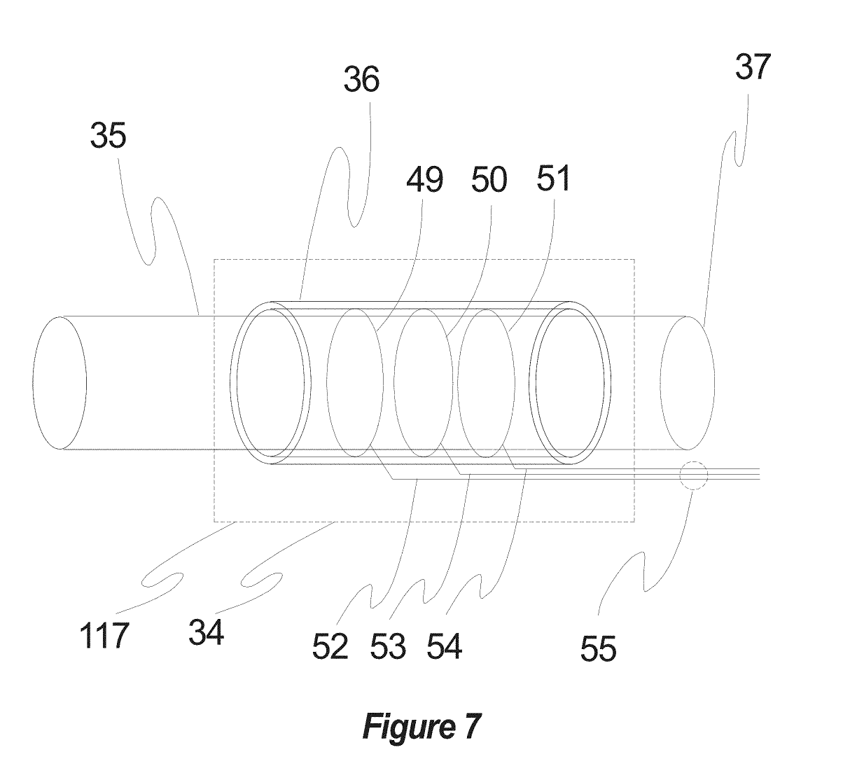

FIG. 7 depicts epineural cuff electrode neuromodulatory interface 117, one of several designs for neuromodulatory interface 34 included in the present invention. Nerve 35 is shown inserted through nerve cuff 36. For selective afferent stimulation, the nerve 35 is transected distal to the epineural cuff electrode neuromodulatory interface 117. This case is depicted here, in which transected nerve end 37 is seen distal to epineural cuff electrode neuromodulatory interface 117. Epineural electrode 49, 50, and 51 are mounted along the inner surface of nerve cuff 36 and in contact or close proximity to nerve 35. Epineural electrode connecting wire 52, 53, 54 are electrically connected on one end to epineural electrode 49, 50, and 51, respectively, and merge together on the other end to form connecting cable 55.

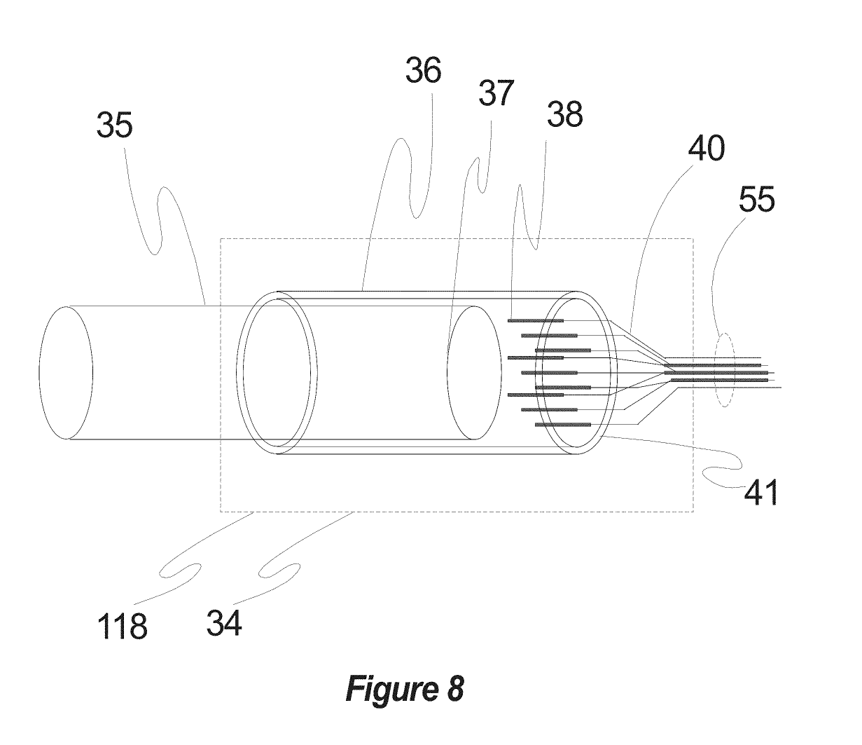

FIG. 8 depicts longitudinal electrode neuromodulatory interface 118, one of several designs for neuromodulatory interface 34 included in the present invention. Nerve 35 is shown inserted into nerve cuff 36. For selective afferent stimulation, the nerve 35 is transected prior to surgical insertion into nerve cuff 36. Longitudinal electrode array 38 is mounted within nerve cuff 36 and in contact or close proximity to nerve 35. Connecting wire array 40 provides electrical connection from each element of longitudinal electrode array 38 to connecting cable 55. Nerve cuff end plate 41 is attached to the distal end of nerve cuff 36. Nerve 35 may be advanced sufficiently far into longitudinal electrode array 38 such that elements of longitudinal electrode array 38 penetrate into nerve 35. Alternatively, nerve 35 may be placed with a gap between transected nerve end 37 and longitudinal electrode array —38 such that neural regeneration occurs from transected nerve end 37 toward and in close proximity to elements of longitudinal electrode array 38.

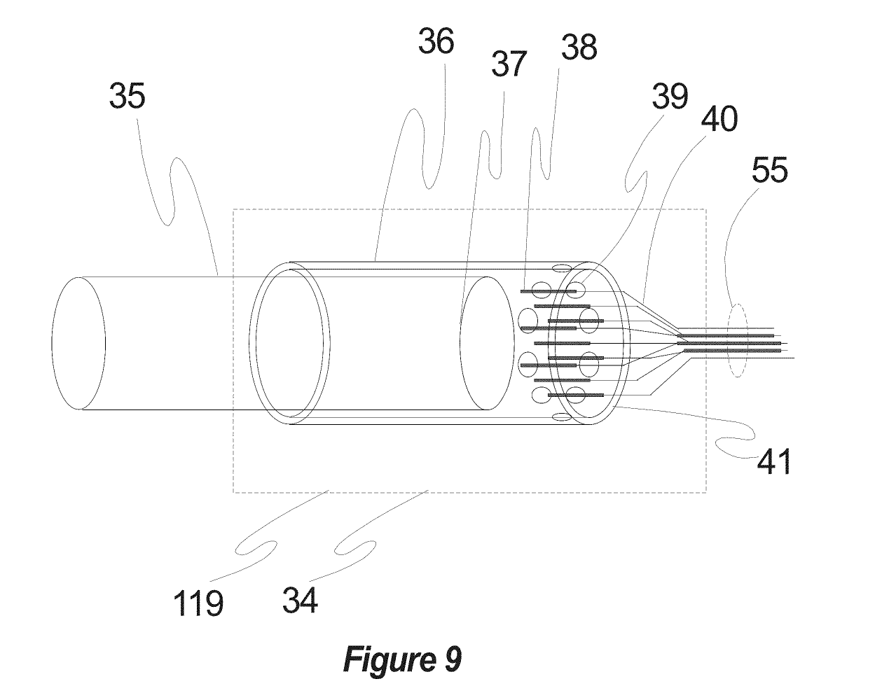

FIG. 9 depicts longitudinal electrode regeneration port neuromodulatory interface 119, an improved design for neuromodulatory interface 34 included in the present invention. Nerve 35 is shown inserted into nerve cuff 36. For selective afferent stimulation, the nerve 35 is transected prior to surgical insertion into nerve cuff 36. Longitudinal electrode array 38 is mounted within nerve cuff 36 and in contact or close proximity to nerve 35. Connecting wire array 40. provides electrical connection from each element of longitudinal electrode array 38 to connecting cable 55. Nerve cuff end plate 41 is attached to the distal end of nerve cuff 36. Nerve 35 may be advanced sufficiently far into longitudinal electrode array 38 such that elements of longitudinal electrode array 38 penetrate into nerve 35. Alternatively, nerve 35 may be placed with a gap between transected nerve end 37 and longitudinal electrode array 38 such that neural regeneration occurs from transected nerve end 37 toward and in close proximity to elements of longitudinal electrode array 38. At least one of nerve cuff 36 and nerve cuff end plate 41 are perforated with one or a multiplicity of regeneration port 39 to facilitate and enhance regeneration of nerve fibers from transected nerve end 37.

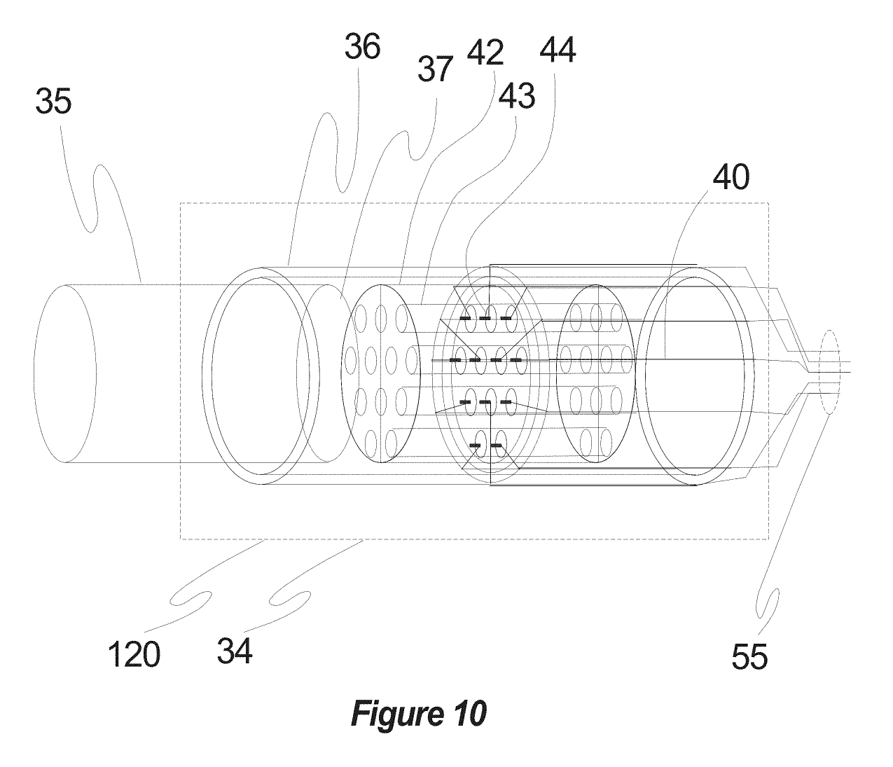

FIG. 10 depicts regeneration tube neuromodulatory interface 120, an advanced design for neuromodulatory interface 34 included in the present invention. Nerve 35 is shown inserted into nerve cuff 36. For selective afferent stimulation, the nerve 35 is transected prior to surgical insertion into nerve cuff 36. Regeneration electrode array 44 is mounted within regeneration tube array 42, which is contained within nerve cuff 36. Each regeneration tube 43 contains at least one element of regeneration electrode array 44. Each element of regeneration electrode array 44 is electrically connected by at least one element of connecting wire array 40 to connecting cable —55. Nerve 35 may be surgically inserted into nerve cuff 36 sufficiently far to be adjacent to regeneration tube array —42 or may be placed with a gap between transected nerve end 37 and regeneration tube array 42. Neural regeneration occurs from transected nerve end 37 toward and through regeneration tube 43 elements regeneration tube array 42.

The present invention further includes stimulation of other tissues that influence vagus nerve activity. These include tissues of the esophagus, stomach, small and large intestine, pancreas, liver, gallbladder, kidney, mesentery, appendix, bladder, uterus, and other intraabdominal tissues. Stimulation of one or a multiplicity of these tissues modulates activity of the vagus nerve afferent fibers without significantly altering activity of efferent fibers. This method and the associated apparatus facilitate the stimulation of vagus nerve afferent fibers without activating vagus nerve efferent fibers, thereby overcoming the ulcerogenic and cardiac side effects of nonselective vagus nerve stimulation. This represents a major advance in vagus nerve modulation and overcomes the potentially life-threatening complications of nonselective stimulation of the vagus nerve.

A4b. Parasympathetic Stimulation—Other. The present invention teaches stimulation of the cervical nerves or their roots or branches for modulation of the parasympathetic nervous system. Additionally, the present invention teaches stimulation of the sacral nerves or their roots or branches for modulation of the parasympathetic nervous system.

A5. Multichannel Satiety Modulation. FIG. 6 depicts apparatus and method for performing multichannel modulation of satiety. Implantable pulse generator 1 is attached to stomach 8, via attachment means 6 and 7 connected from stomach 8 to attachment fixture 4 and 5, respectively. Implantable pulse generator 1 is electrically connected via modulator cable 32 to modulators 24, 25, 26, 27, 28, and 29, which are affixed to the stomach 8 preferably along the region of the greater curvature of stomach 10. Implantable pulse generator 1 is additionally electrically connected via modulator cable 33 to modulators 30 and 31, which are affixed to the stomach 8 preferably along the region of the lesser curvature of stomach 17. Implantable pulse generator 1 is furthermore electrically connected via modulator cable 18 and 19 to modulators 2 and 3, respectively, which are affixed to the gastric pylorus 12. Modulator 2 is affixed to gastric pylorus via modulator attachment fixture 22 and 23, and modulator 3 is affixed to gastric pylorus via modulator attachment fixture 20 and 21.

Using the apparatus depicted in FIG. 6, satiety modulation is achieved through multiple modalities. A multiplicity of modulators, including modulator 30 and 31 facilitate stimulation of vagus and sympathetic afferent fibers directly, as well as through stimulation of tissues, including gastric muscle, that in turn influence activity of the sympathetic and vagus afferent fibers. A multiplicity of modulators, including modulator 24, 25, 26, 27, 28, and 29 facilitate stimulation of sympathetic afferent fibers directly, as well as through stimulation of tissues, including gastric muscle, that in turn influence activity of the sympathetic fibers. Any of these modulators may be used to modulate vagus nerve activity; however, one advancement taught in the present invention is the selective stimulation of sympathetic nerve fiber activation, and this is facilitated by modulators 24, 25, 26, 27, 28, and 29, by virtue of their design for and anatomical placement in regions of the stomach 8 that are not innervated by the vagus nerve or its branches.

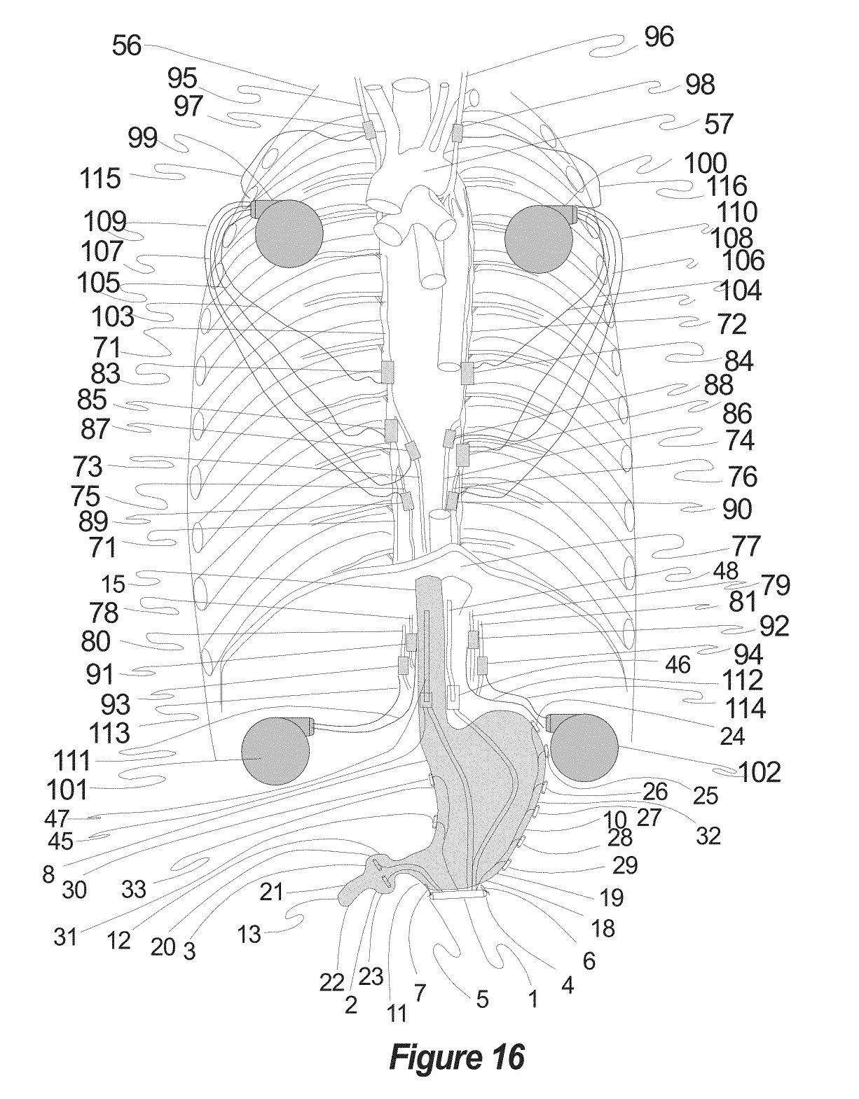

In addition to the apparatus and methods depicted in FIG. 6 for satiety modulation, the present invention further includes satiety modulation performed with the apparatus depicted in FIG. 16, and described previously, using stimulation of right sympathetic trunk 71, left sympathetic trunk 72, right greater splanchnic nerve 73, left greater splanchnic nerve 74, right lesser splanchnic nerve 75, left lesser splanchnic nerve 76 or other branch or the sympathetic nervous system.

B. Metabolic Modulation B.1. Sympathetic Efferent Stimulation. One objective of the modulator configuration employed in the present invention is the selected stimulation of sympathetic efferent nerve fibers. The present invention includes a multiplicity of potential modulator configurations and combinations of thereof. The present embodiment includes modulators placed at a combination of sites to interface with the sympathetic efferent fibers. These sites include the musculature of the stomach, the distal sympathetic branches penetrating into the stomach, postganglionic axons and cell bodies, preganglionic axons and cell bodies, the sympathetic chain and portions thereof, the intermediolateral nucleus, the locus ceruleus, the hypothalamus, and other structures comprising or influencing activity of the sympathetic nervous system.

Stimulation of the sympathetic efferents is performed to elevate the metabolic rate and lipolysis in the adipose tissue, thereby enhancing breakdown of fat and weight loss in the patient.

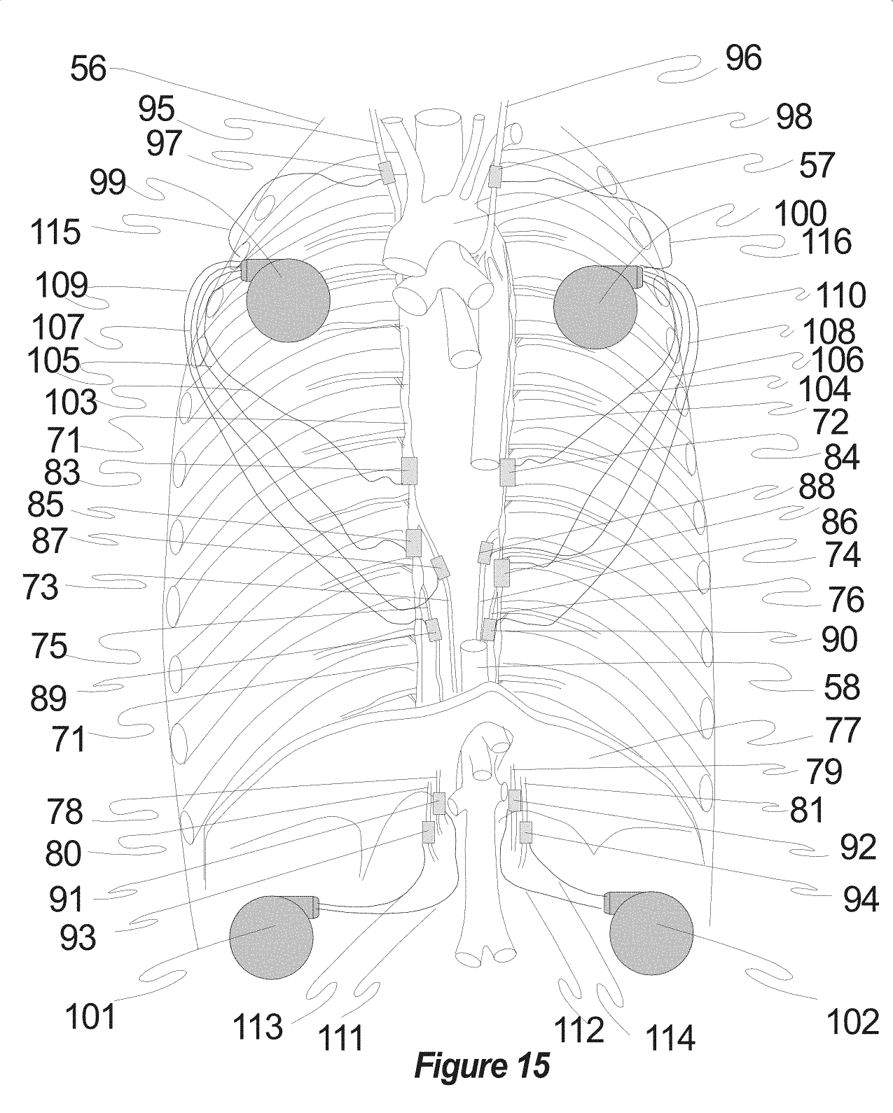

B.1.a. Sympathetic Efferent Stimulation Sympathetic Trunk. FIGS. 14, 15, and 16 depict apparatus for stimulation of the sympathetic nervous system. FIG. 14 depicts a subset of anatomical locations for placement of neuromodulatory interfaces for modulation of the sympathetic nervous system. FIG. 15 depicts the same apparatus with the further addition of a set of implantable pulse generator 1 and connecting cables. FIG. 16 depicts the apparatus shown in FIG. 15 with the further addition of gastric modulation apparatus also depicted in FIG. 6.

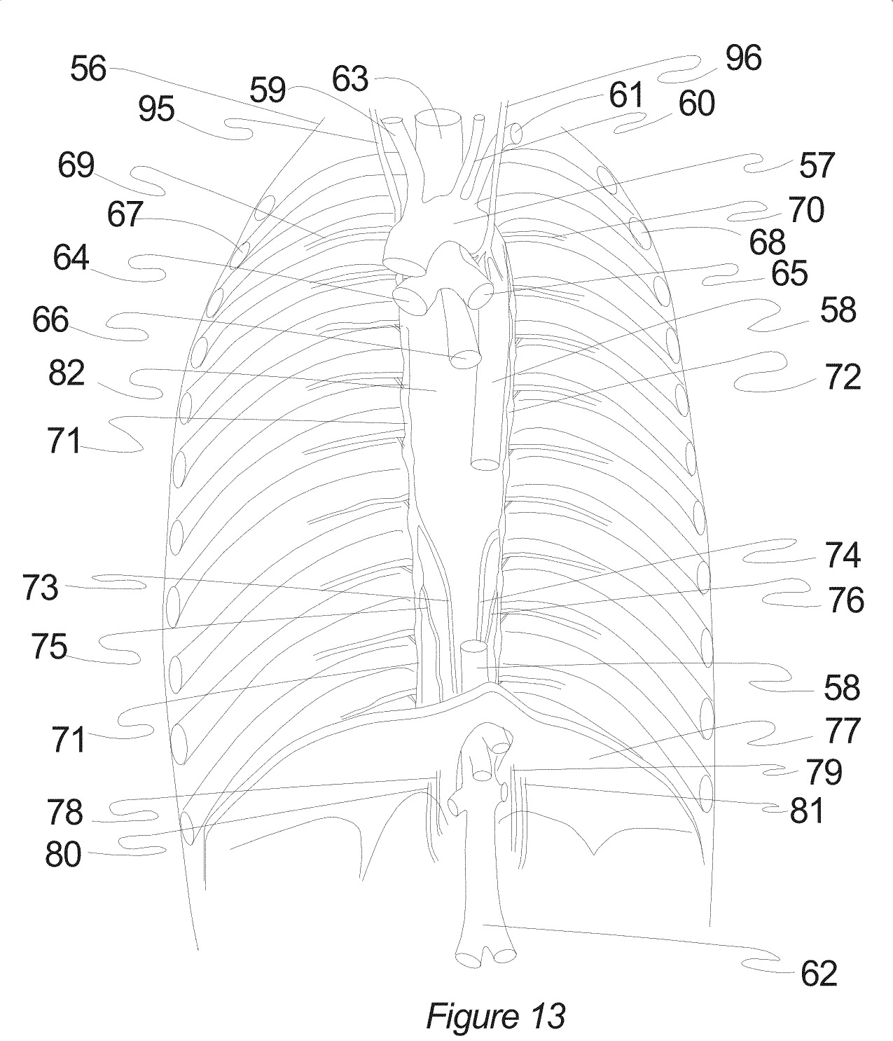

FIG. 13 reveals the normal anatomy of the thoracic region. Trachea 63 is seen posterior to aortic arch 57. Brachiocephalic artery 59, left common carotid artery 60 arise from aortic arch 57, and left subclavian artery 61 arises from the left common carotid artery 60. Right mainstem bronchus 64 and left mainstem bronchus —65 arise from trachea 63. Thoracic descending aorta 58 extends from aortic arch 57 and is continuous with abdominal aorta 62. Right vagus nerve 95 and left vagus nerve 96 are shown. Intercostal nerve 69 and 70 are shown between respective pairs of ribs, of which rib 67 and rib 68 are labeled.

Right sympathetic trunk 71 and left sympathetic trunk are lateral to mediastinum 82. Right greater splanchnic nerve 73 and right lesser splanchnic nerve 75 arise from right sympathetic trunk 71. Left greater splanchnic nerve 74 and left lesser splanchnic nerve 76 arise from left sympathetic trunk 72. Right subdiaphragmatic greater splanchnic nerve 78, left subdiaphragmatic greater splanchnic nerve 79, right subdiaphragmatic lesser splanchnic nerve 80, and left subdiaphragmatic lesser splanchnic nerve 81 are extensions below the diaphragm 77 of the right greater splanchnic nerve 73, left greater splanchnic nerve 74, right lesser splanchnic nerve 75, and left lesser splanchnic nerve 76, respectively.

B. 1.b. Sympathetic Efferent Stimulation—Splanchnic Nerves. FIG. 14 depicts multichannel sympathetic modulation implanted with relevant anatomical structures. Sympathetic trunk neuromodulatory interface 83 and 85 are implanted adjacent to and in communication with right sympathetic trunk 71. Sympathetic trunk neuromodulatory interface 84 and 86 are implanted adjacent to and in communication with left sympathetic trunk 72. Sympathetic trunk neuromodulatory interface 83, 84, 85, and 86 are implanted superior to their respective sympathetic trunk levels at which the right greater splanchnic nerve 73, left greater splanchnic nerve 74, right lesser splanchnic nerve 75, and left lesser splanchnic nerve 76, arise, respectively.

Thoracic splanchnic nerve interface 87, 88, 89, 90 are implanted adjacent to and in communication with the right greater splanchnic nerve 73, left greater splanchnic nerve 74, right lesser splanchnic nerve 75, and left lesser splanchnic nerve 76, arise, respectively. Abdominal splanchnic nerve interface 91, 92, 93, and 94 are implanted adjacent to and in communication with the right subdiaphragmatic greater splanchnic nerve 78, left subdiaphragmatic greater splanchnic nerve 79, right subdiaphragmatic lesser splanchnic nerve 80, and left subdiaphragmatic lesser splanchnic nerve 81, respectively.

Stimulation of at least one of right sympathetic trunk 71, left sympathetic trunk 72, right greater splanchnic nerve 73, left greater splanchnic nerve 74, right lesser splanchnic nerve 75, and left lesser splanchnic nerve 76, right subdiaphragmatic greater splanchnic nerve 78, left subdiaphragmatic greater splanchnic nerve 79, right subdiaphragmatic lesser splanchnic nerve 80, and left subdiaphragmatic lesser splanchnic nerve 81 enhances metabolism of adipose tissue. Stimulation of these structures may be performed using at least one of electrical energy, electrical fields, optical energy, mechanical energy, magnetic energy, chemical compounds, pharmacological compounds, thermal energy, vibratory energy, or other means for modulating neural activity.

FIG. 15 depicts the implanted neuromodulatory interfaces as in FIG. 14, with the addition of the implanted pulse generators. Implantable pulse generator 99 is connected via connecting cable 103, 105, 107, —109, 115, to sympathetic trunk neuromodulatory interface 83. and 85, and thoracic splanchnic neuromodulatory interface 87 and 89, and vagus neuromodulatory interface 97, respectively. Implantable pulse generator 100 is connected via connecting cable 104, 106, 108, 110, 116, to sympathetic trunk neuromodulatory interface 83. and 85, and thoracic splanchnic neuromodulatory interface 88 and 90, and vagus neuromodulatory interface 98, respectively. Implantable pulse generator 101 is connected via connecting cable 111 and 113 to abdominal splanchnic neuromodulatory interface 91 and 93, respectively. Implantable pulse generator 102 is connected via connecting cable 112 and 114 to abdominal splanchnic neuromodulatory interface 92 and 94, respectively.

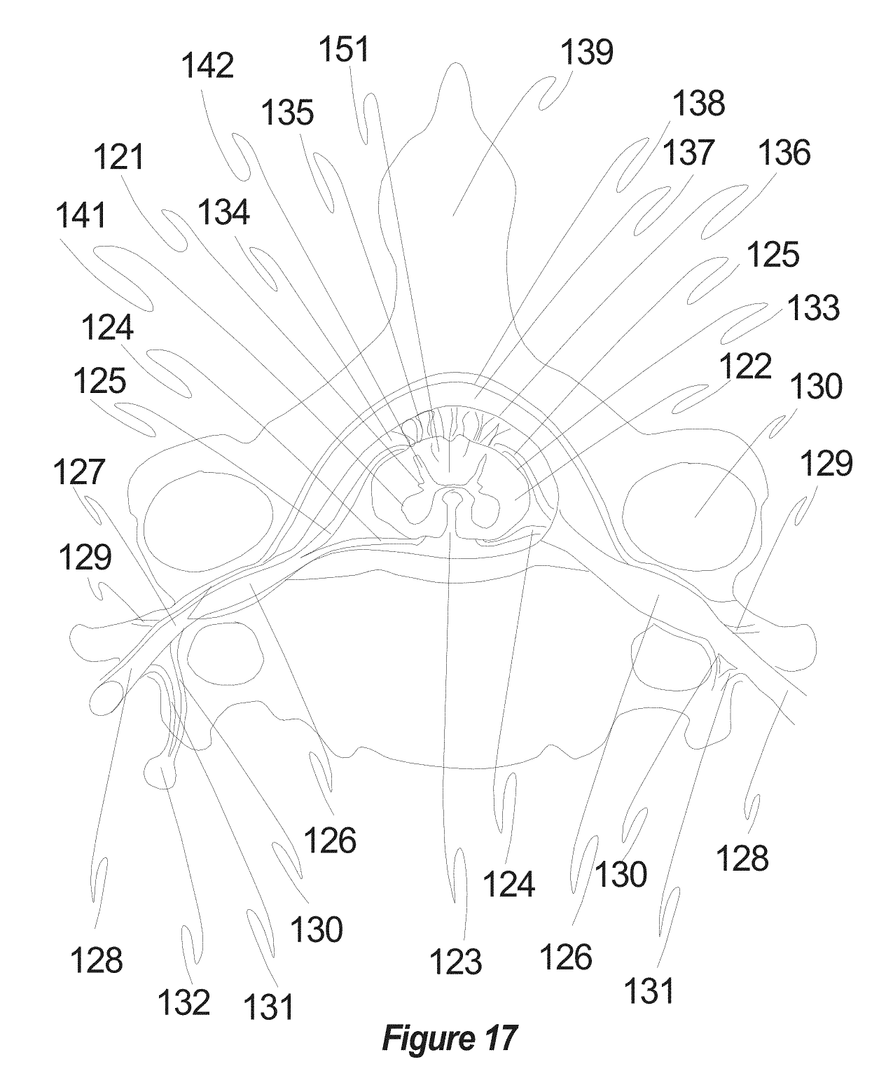

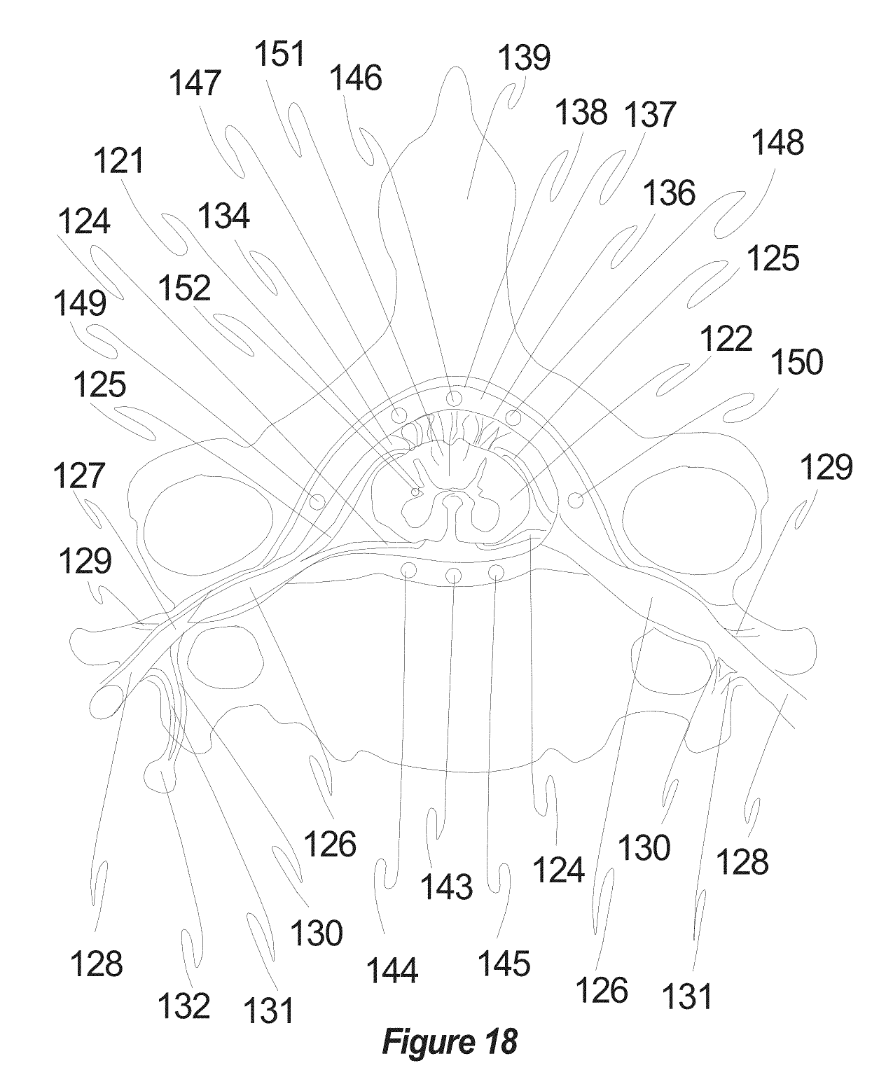

B.1.c. Sympathetic Efferent Stimulation—Spinal Cord. FIGS. 17 and 18 depicts the normal cross-sectional anatomy of the spinal cord 151 and anatomy with implanted neuromodulatory interfaces, respectively.

FIG. 17 depicts the normal anatomical structures of the spinal cord 151, including several of its component structures such as the intermediolateral nucleus 121, ventral horn of spinal gray matter 141, dorsal horn of spinal gray matter 142, spinal cord white matter 122, anterior median fissure 123. Other structures adjacent to or surrounding spinal cord 151 include ventral spinal root 124, dorsal spinal root 125, spinal ganglion 126, spinal nerve 127, spinal nerve anterior ramus 128, spinal nerve posterior ramus 129, gray ramus communicantes 130, white ramus communicantes 131, sympathetic trunk 132, pia mater 133, subarachnoid space 134, arachnoid 135, meningeal layer of dura mater 136, epidural space 137, periosteal layer of dura mater 138, and vertebral spinous process 139, and vertebral facet 140.

FIG. 17 depicts the normal anatomy of the spinal cord seen in transverse section. Spinal cord and related neural structures include intermediolateral nucleus 121, spinal cord white matter 122, anterior median fissure 123, ventral spinal root 124, dorsal spinal root 125, spinal ganglion 126, spinal nerve 127, spinal nerve anterior ramus 128, spinal nerve posterior ramus 129, grey ramus communicantes 130, white ramus communicantes 131, sympathetic trunk 132, pia mater 133, subarachnoid space 134, arachnoid 135, meningeal layer of dura 136, epidural space 137, periosteal layer of dura mater 138, vertebral spinous process 139, vertebral facet 140, ventral horn of spinal gray matter 141, and dorsal horn of spinal gray matter 142.

FIG. 18 depicts the spinal neuromodulatory interfaces positioned in the vicinity of spinal cord —151. Neuromodulatory interfaces positioned anterior to spinal cord 151 include anterior central spinal neuromodulatory interface 143, anterior right lateral spinal neuromodulatory interface 144, and anterior left lateral spinal neuromodulatory interface 145. Neuromodulatory interfaces positioned posterior to spinal cord 151 include posterior central spinal neuromodulatory interface 146, posterior right lateral spinal neuromodulatory interface 147, and posterior left lateral spinal neuromodulatory interface 148. Neuromodulatory interfaces positioned lateral to spinal cord 151 include right lateral spinal neuromodulatory interface 149 and left lateral spinal neuromodulatory interface 150. Neuromodulatory interfaces positioned within the spinal cord 151 include intermediolateral nucleus neuromodulatory interface 152.

Stimulation, inhibition, or other modulation of the spinal cord 151 is used to modulate fibers of the sympathetic nervous system, including those in the intermediolateral nucleus 121 and efferent and efferent fibers connected to the intermediolateral nucleus 121. Modulation of at least one of portions of the spinal cord 151, intermediolateral nucleus 121, ventral spinal root 124, dorsal spinal root 125, spinal ganglion 126, spinal nerve 127, gray ramus communicantes 130, white ramus communicantes 131 and other structures facilitates modulation of activity of the sympathetic trunk 132. Modulation of activity of the sympathetic trunk 132, in turn, is used to modulate at least one of metabolic activity, satiety, and appetite. This may be achieved using intermediolateral nucleus neuromodulatory interface 152, placed in or adjacent to the intermediolateral nucleus 121. The less invasive design employing neuromodulatory interfaces (144, 145, 146, 147, 148, 149, 150) shown positioned in the in epidural space 137 is taught in the present invention.

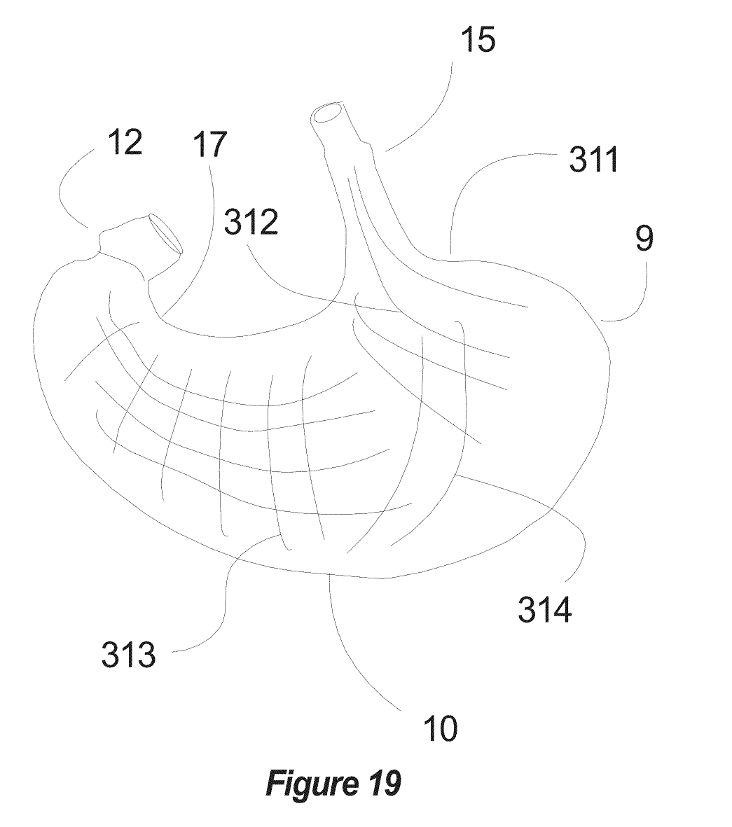

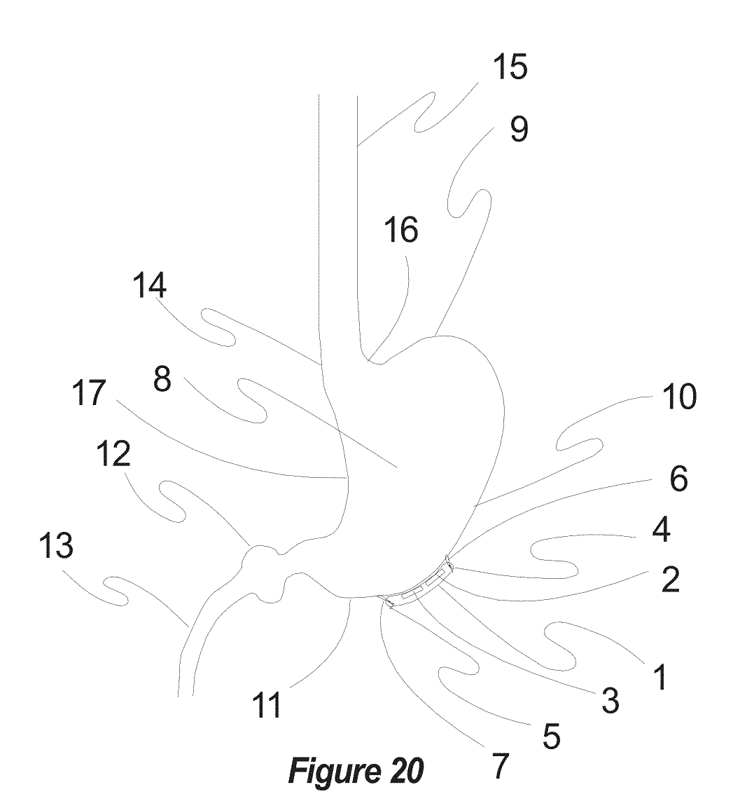

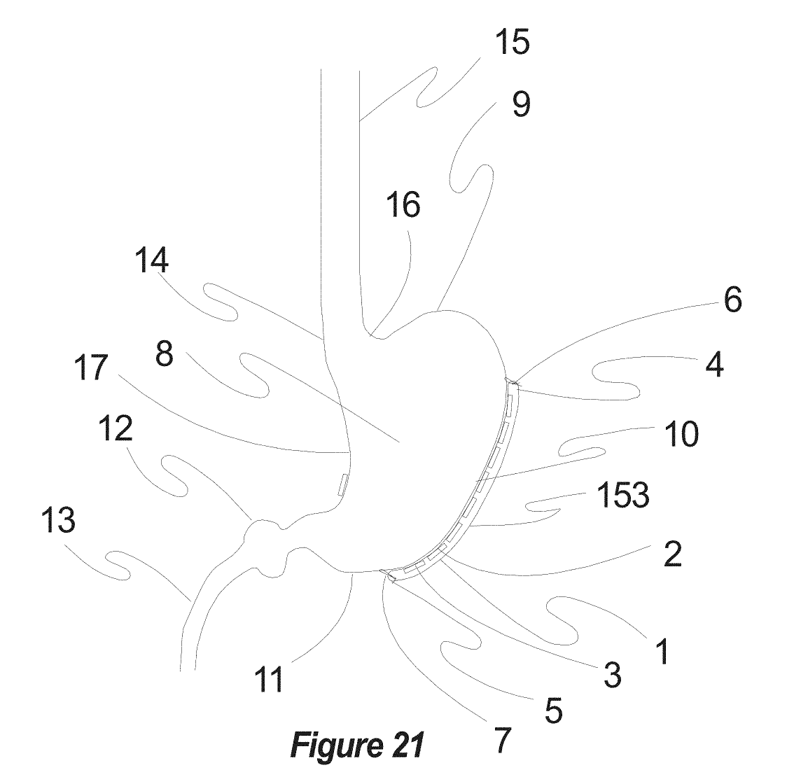

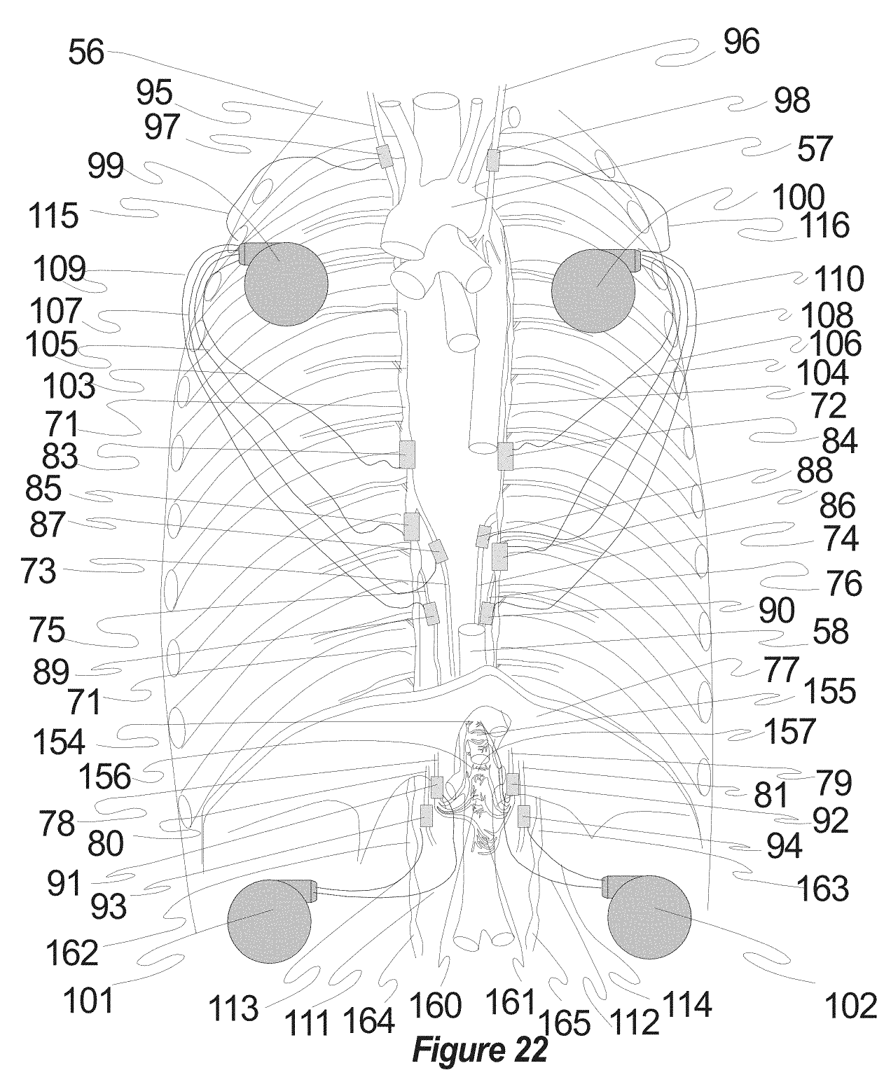

FIG. 19 depicts a cut away view of the stomach, revealing the four coats: serous, muscular, areolar, and mucous. The gastric muscular coat 311 is comprised of 3 layers, the gastric longitudinal fibers 311, gastric circular fibers 312, and gastric oblique fibers 313. Gastric longitudinal fibers 311 are most superficial; they are continuous with the longitudinal fibers of the esophagus 15, radiating in a stellate manner from the cardiac orifice. They are most distinct along the curvatures, especially the lesser, but are very thinly distributed over the surfaces. At the pyloric end, they are more thickly distributed and are continuous with the longitudinal fibers of the small intestine. Gastric circular fibers 313 form a uniform layer over the whole extent of the stomach beneath the gastric longitudinal fibers 311. At the gastric pylorus 12 they are most abundant and are aggregated into a circular ring, which projects into the lumen and forms, with the fold of mucous membrane covering its surface, the pyloric valve. They are continuous with the circular layers of the esophagus 15. The gastric oblique fibers 314 are beneath the gastric circular fibers 313. Stimulation of afferent neural fibers innervating stretch receptors in these muscle layers is taught in the parent case. This figure merely depicts anatomical detail.

B.1.d. Sympathetic Efferent Stimulation—Other. The present invention further includes modulation of all sympathetic efferent nerves, nerve fibers, and neural structures. These sympathetic efferent neural structures include but are not limited to distal sympathetic nerve branches, mesenteric nerves, sympathetic efferent fibers at all spinal levels, rami communicantes at all spinal levels, paravertebral nuclei, prevertebral nuclei, and other sympathetic structures.

B.2. Noninvasive Stimulation. The present invention teaches a device for metabolic control using tactile stimulation. Tactile stimulation of afferent neurons causes alterations in activity of sympathetic neurons which influence metabolic activity of adipose tissue. The present invention teaches tactile stimulation of skin, dermal and epidermal sensory structures, subcutaneous tissues and structures, and deeper tissues to modulate activity of afferent neurons.

This device for metabolic control employs vibratory actuators. Alternatively, electrical stimulation, mechanical stimulation, optical stimulation, acoustic stimulation, pressure stimulation, and other forms of energy that modulate afferent neural activity, are used.

C. Multimodal Metabolic Modulation. To maximize efficacy while tailoring treatment to minimize side effects, the preferred embodiment includes a multiplicity of treatment modalities, including afferent, efferent, and neuromuscular modulation.

Afferent signals are generated to simulate satiety. This is accomplished through neural, neuromuscular, and hydrostatic mechanisms. Electrical stimulation of the vagus via vagus nerve interface 45 afferents provides one such channel to transmit information to the central nervous system for the purpose of eliciting satiety. Electrical stimulation of the sympathetic afferents via sympathetic nerve interface 46 provides another such channel to transmit information to the central nervous system for the purpose of eliciting satiety. Electrical stimulation of gastric circular muscle layer may also be employed to induce satiety.

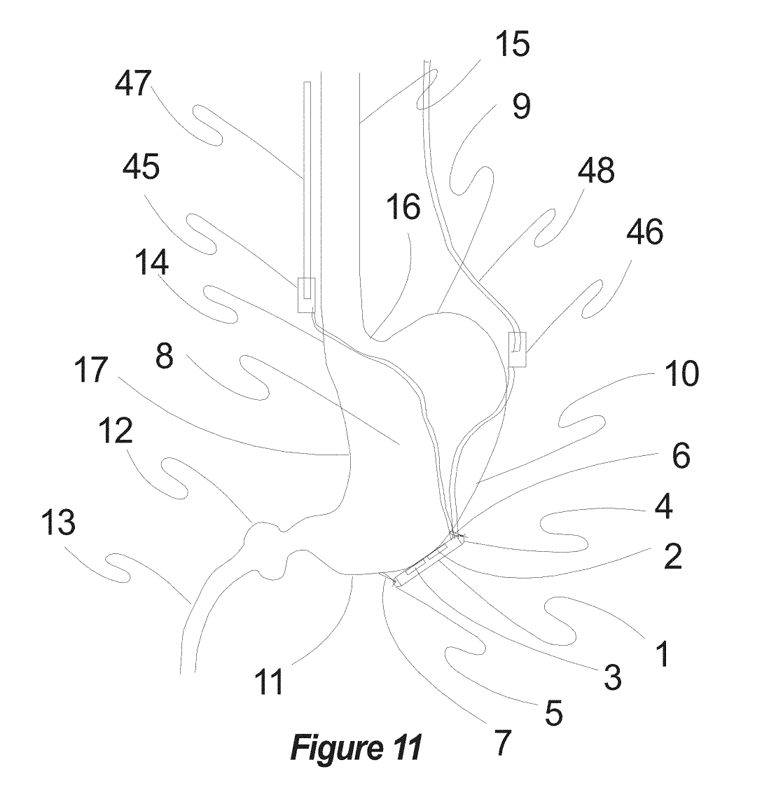

In FIG. 11, multimodal stimulation is depicted, including stimulation of gastric musculature using modulators 2 and 3, as well as stimulation of afferent fibers of the proximal stump of vagus nerve 47 using vagus nerve modulator 45 and stimulation of afferent fibers of sympathetic nerve branch 48.

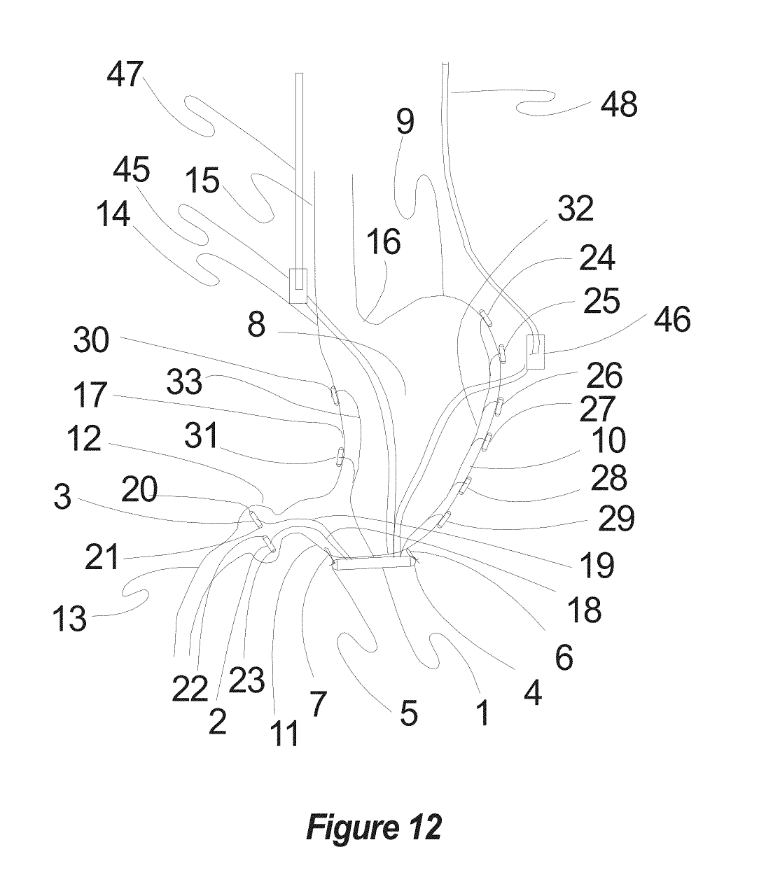

In FIG. 12, expanded multimodal stimulation is depicted, including those modalities shown in FIG. 11, including stimulation of gastric musculature using modulators 2 and 3, as well as stimulation of afferent fibers of the proximal stump of vagus nerve 47 using vagus nerve modulator 45 and stimulation of afferent fibers of sympathetic nerve branch 48., in addition to those modalities shown in FIG. 6, explained in detail above, including modulation of gastric muscular fibers, sympathetic afferent fibers innervating gastric tissues, and vagus afferent fibers innervating gastric tissues.

In FIG. 16, further expanded multimodal modulation is depicted, including modalities encompassed and described above and depicted in FIG. 15 and FIG. 12. This includes modulation of gastric muscle fibers, fibers of the sympathetic nerve branch 48 and vagus nerve 47 that innervate gastric tissues, and a multiplicity of structures in the sympathetic nervous system and vagus nerve 47.

E. System/Pulse Generator Design. Neuromodulatory interfaces that use electrical energy to modulate neural activity may deliver a broad spectrum of electrical waveforms. One preferred set of neural stimulation parameter sets includes pulse frequencies ranging from 0.1 Hertz to 1000 Hertz, pulse widths from 1 microsecond to 500 milliseconds. Pulses are charge balanced to insure no net direct current charge delivery. The preferred waveform is bipolar pulse pair, with an interpulse interval of 1 microsecond to 1000 milliseconds. Current regulated stimulation is preferred and includes pulse current amplitudes ranging from 1 microamp to 1000 milliamps. Alternatively, voltage regulation may be used, and pulse voltage amplitudes ranging from 1 microamp to 1000 milliamps. These parameters are provided as exemplary of some of the ranges included in the present invention; variations from these parameter sets are included in the present invention.

FIG. 22 shows the same invention taught in the parent case. In this figure, the distal portion of the sympathetic nervous system is shown in more detail. In the parent case, modulation of the sympathetic nervous system was taught for the treatment of disease. When a portion of the nervous system is modulated, connected neural structures are likewise modulated. Neural structures proximal and distal to the location of the modulator are modulated by the action of the modulator. A multiplicity of locations for neuromodulators are presented in the parent case, and other locations may be selected without departing from the parent case invention. The addition of more detail of the nervous system renders obvious to the reader of the parent application additional locations for placement of neural modulators.

In FIG. 22, additional anatomical structures shown include celiac plexus 154, celiac ganglion 155, superior mesenteric plexus 156, superior mesenteric ganglion 157, renal plexus 158, renal ganglion 159, inferior mesenteric plexus 160, iliac plexus 161, right lumbar sympathetic ganglia 162, left lumbar sympathetic ganglia 163, right sacral sympathetic ganglia 164, and left sacral sympathetic ganglia 165.

It is obvious to the reader that modulation of the right greater splanchnic nerve 73, the performance of which is exemplified by Abdominal Splanchnic Neuromodulatory Interface 91, will in turn effect modulation of connected structures, including proximal and distal portions of Right Subdiaphragmatic Greater Splanchnic Nerve 78. Proximal or retrograde conduction of neural signals will effect modulation of Right Greater Splanchnic Nerve 73 and more proximal structures. Distal or anterograde conduction of neural signals will effect modulation of distal structures including but not limited to celiac plexus 154, celiac ganglion 155, superior mesenteric plexus 156, superior mesenteric ganglion 157, renal plexus 158, renal ganglion 159, inferior mesenteric plexus 160, iliac plexus 161, and other structures connected by neural pathways.

It is obvious to the reader that modulation of the left greater splanchnic nerve 74, the performance of which is exemplified by Abdominal Splanchnic Neuromodulatory Interface 92, will in turn effect modulation of connected structures, including proximal and distal portions of Left Subdiaphragmatic Greater Splanchnic Nerve 79. Proximal or retrograde conduction of neural signals will effect modulation of Left Greater Splanchnic Nerve 74 and more proximal structures. Distal or anterograde conduction of neural signals will effect modulation of distal structures including but not limited to celiac plexus 154, celiac ganglion 155, superior mesenteric plexus 156, superior mesenteric ganglion 157, renal plexus 158, renal ganglion 159, inferior mesenteric plexus 160, iliac plexus 161, and other structures connected by neural pathways.

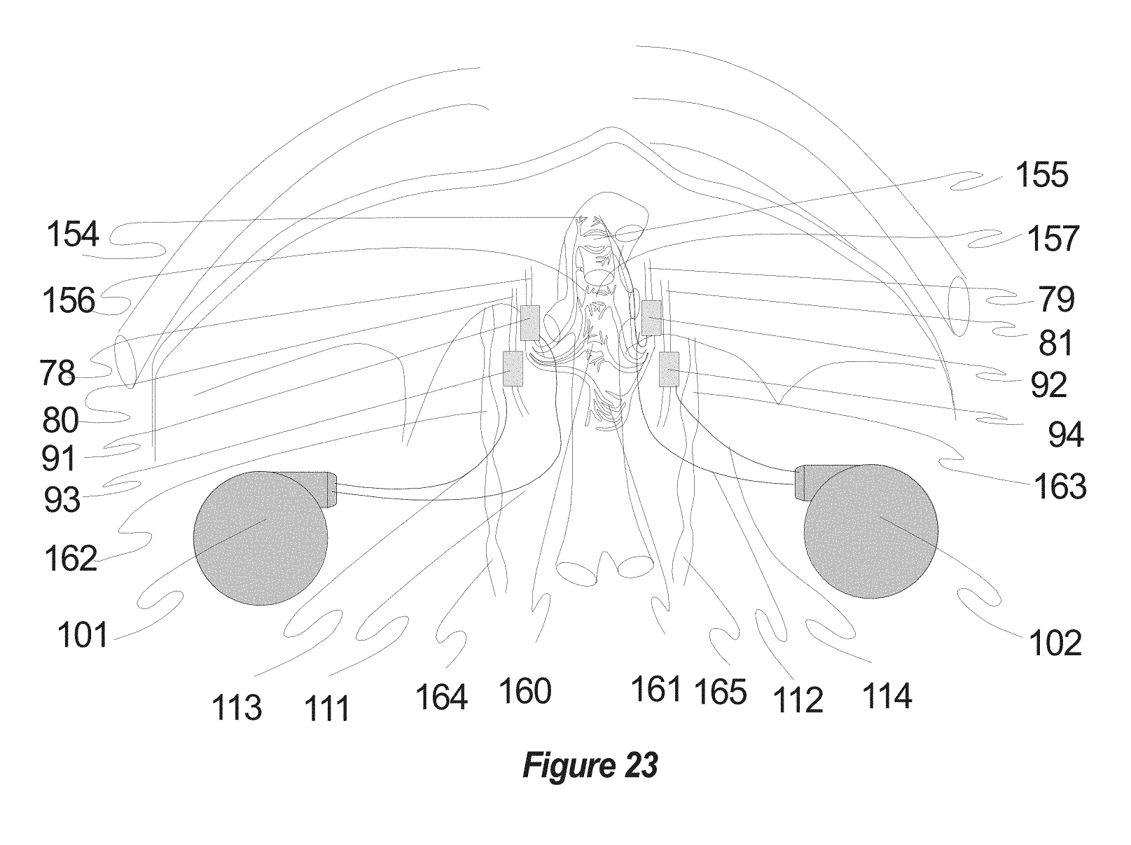

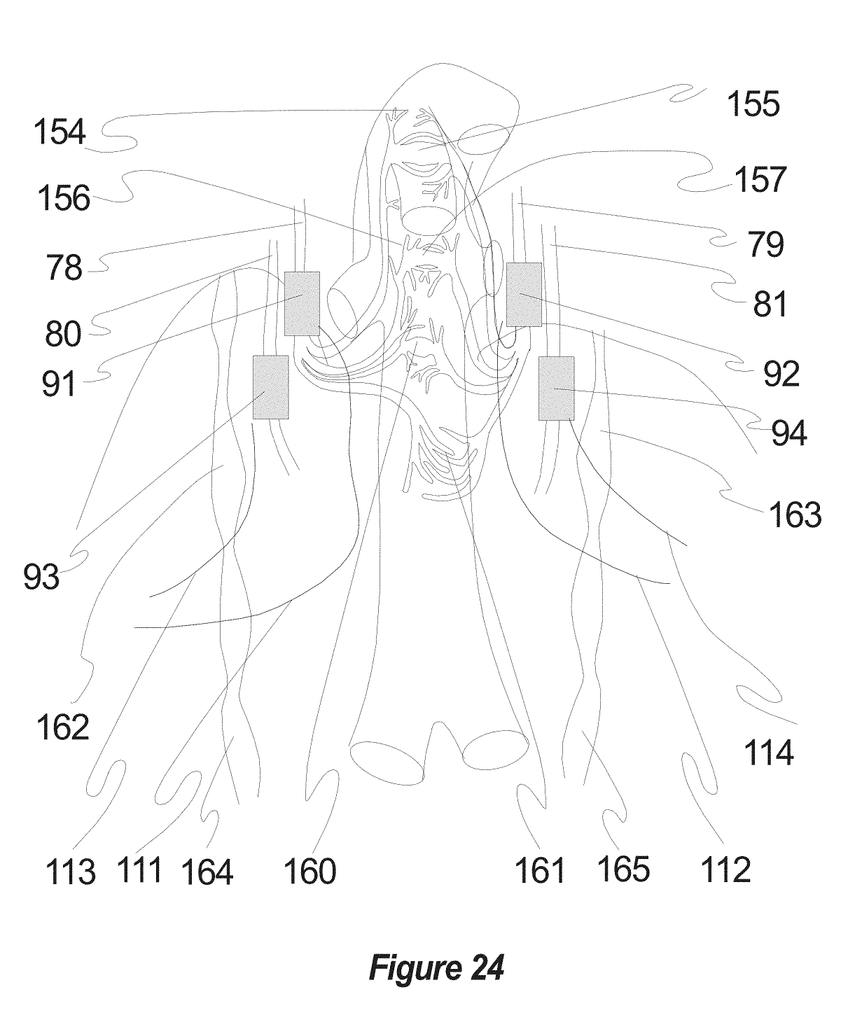

FIG. 23 and FIG. 24 show Abdominal Splanchnic Neuromodulatory Interface 91, Abdominal Splanchnic Neuromodulatory Interface 92, Abdominal Splanchnic Neuromodulatory Interface 93, Abdominal Splanchnic Neuromodulatory Interface 94 and surrounding anatomical structures, as described above, at larger magnification.

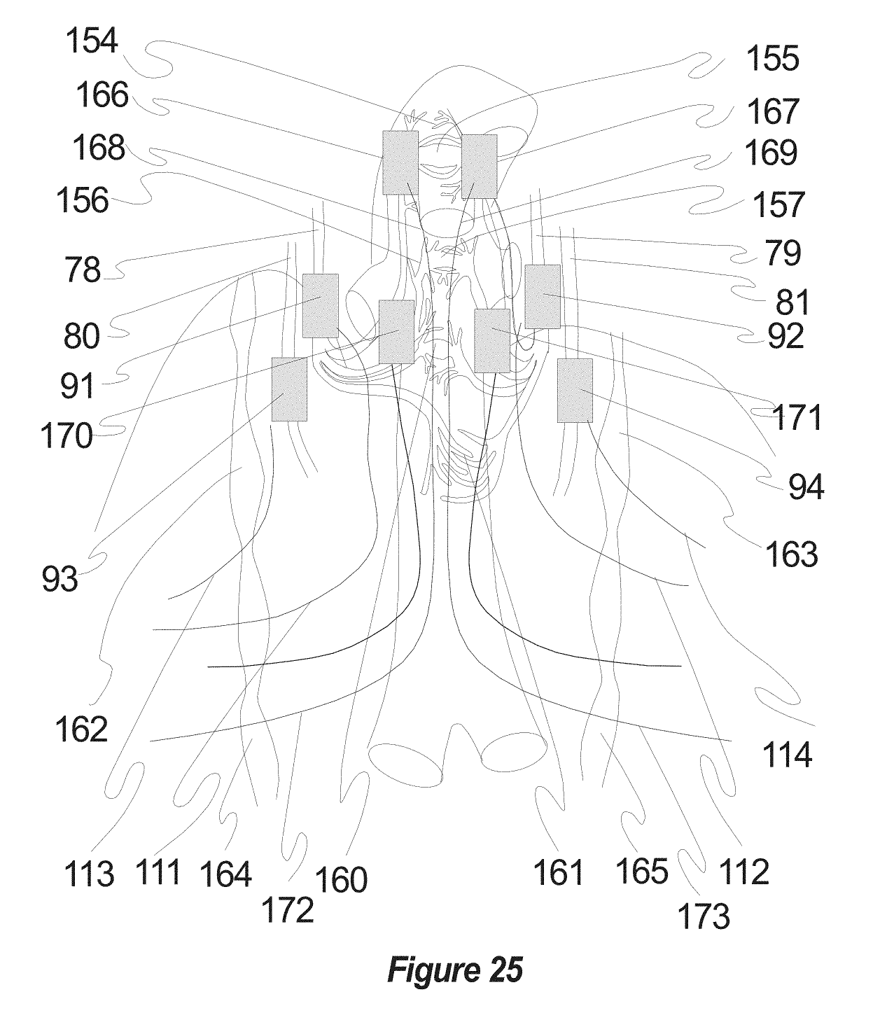

FIG. 25 shows Abdominal Splanchnic Neuromodulatory Interface 166, Abdominal Splanchnic Neuromodulatory Interface 167, Abdominal Splanchnic Neuromodulatory Interface 170, and Abdominal Splanchnic Neuromodulatory Interface 171 in proximity to neural structures distal to and in neural communication with each of the right greater splanchnic nerve 73 and left greater splanchnic nerve 73.

Pulse generator 101 generates neuromodulatory signal which is transmitted by connecting cable 168 to abdominal splanchnic neuromodulatory interface 166, which modulates at least one of celiac plexus 154 and celiac ganglion 155. Implantable Pulse generator 102 generates neuromodulatory signal which is transmitted by connecting cable 169 to abdominal splanchnic neuromodulatory interface 167, which modulates at least one of celiac plexus 154 and celiac ganglion 155.

Pulse generator 101 generates neuromodulatory signal which is transmitted by connecting cable 172 to abdominal splanchnic neuromodulatory interface 170, which modulates at least one of superior mesenteric plexus 156, superior mesenteric ganglion 157, renal plexus 158, renal ganglion 159, inferior mesenteric plexus 160, and iliac plexus 161. Pulse generator 102 generates neuromodulatory signal which is transmitted by connecting cable 173 to abdominal splanchnic neuromodulatory interface 171, which modulates at least one of superior mesenteric plexus 156, superior mesenteric ganglion 157, renal plexus 158, renal ganglion 159, inferior mesenteric plexus 160, and iliac plexus 161.

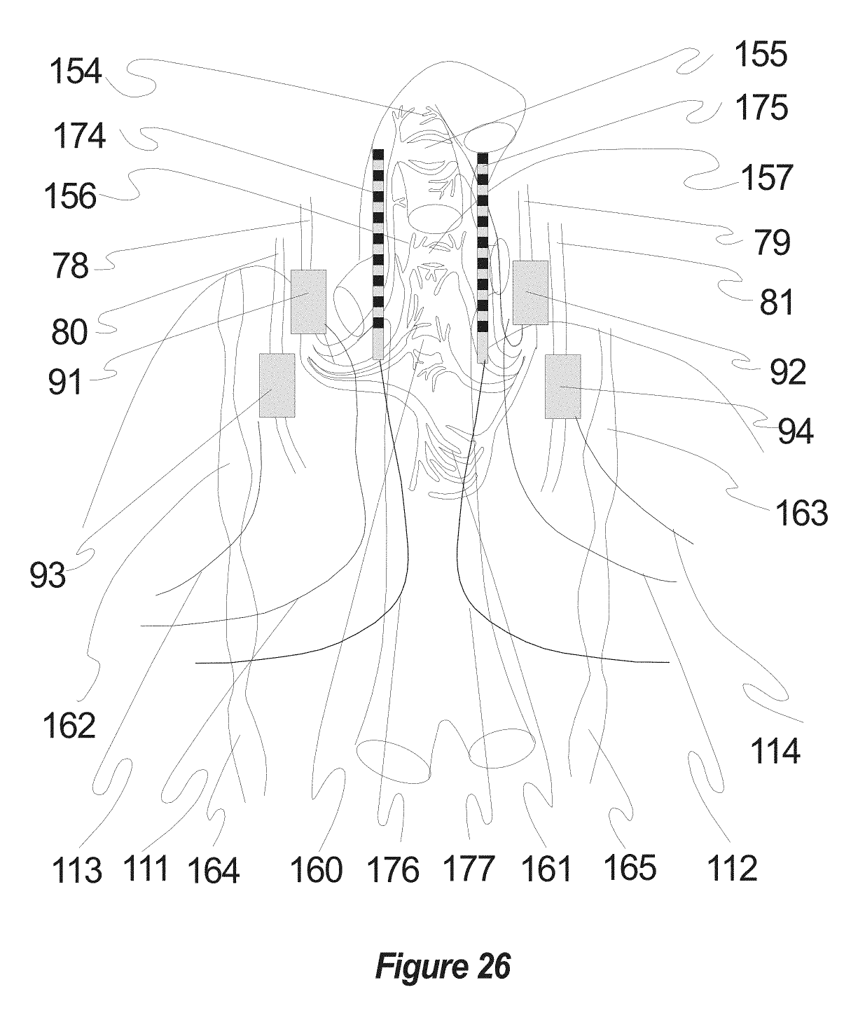

FIG. 26 shows neuromodulator array 174 and neuromodulator array 175 in proximity to neural structures distal to and in neural communication with each of the right greater splanchnic nerve 73 and left greater splanchnic nerve 73.

Pulse generator 101 generates neuromodulatory signal which is transmitted by connecting cable 176 to neuromodulator array 174, which modulates at least one of celiac plexus 154, celiac ganglion 155, superior mesenteric plexus 156, superior mesenteric ganglion 157, renal plexus 158, renal ganglion 159, inferior mesenteric plexus 160, and iliac plexus 161.

Pulse generator 102 generates neuromodulatory signal which is transmitted by connecting cable 177 to neuromodulator array 175, which modulates at least one of celiac plexus 154, celiac ganglion 155, superior mesenteric plexus 156, superior mesenteric ganglion 157, renal plexus 158, renal ganglion 159, inferior mesenteric plexus 160, and iliac plexus 161.

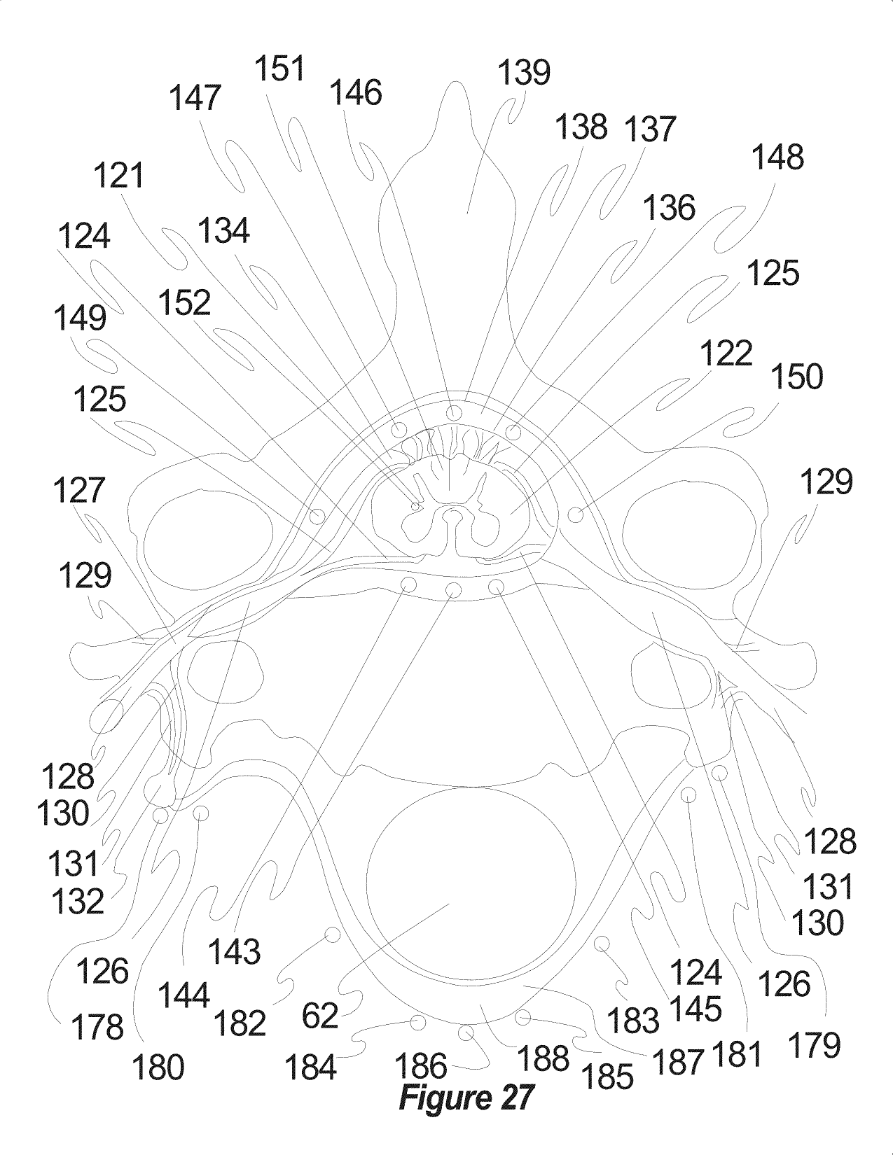

FIG. 27 shows a transverse section through the spinal canal, vertebral columns, and adjacent structures in the lumbar region. The components described may be positioned at a higher level, including cervical and thoracic, or a lower level including sacral and coccygeal, without departing from the present invention. Perispinal neuromodulatory interfaces are described in the description for FIG. 18. Abdominal aorta 62 is shown.

Abdominal Splanchnic Neuromodulatory Interface 178 modulate at least one of sympathetic trunk, 132, Right Lumbar Sympathetic Ganglia 162, and Right Sacral Sympathetic Ganglia 164. Abdominal Splanchnic Neuromodulatory Interface 179 modulates at least one of sympathetic trunk, 132, Left Lumbar Sympathetic Ganglia 163, and Left Sacral Sympathetic Ganglia 165

Abdominal Splanchnic Neuromodulatory Interface 180 modulates at least one neural structure in neural connection to sympathetic trunk 132, including but not limited to right greater splanchnic nerve 73, right lesser splanchnic nerve 75, right least splanchnic nerve, or other structure. Abdominal Splanchnic Neuromodulatory Interface 181 modulates at least one neural structure in neural connection to sympathetic trunk 132, including but not limited to left greater splanchnic nerve 74, left lesser splanchnic nerve 76, left least splanchnic nerve, or other structure.

Abdominal Splanchnic Neuromodulatory Interface 182, Abdominal Splanchnic Neuromodulatory Interface 183, Abdominal Splanchnic Neuromodulatory Interface 184, Abdominal Splanchnic Neuromodulatory Interface 185, and Abdominal Splanchnic Neuromodulatory Interface 186 each modulate abdominal structures including but not limited to celiac plexus 154, celiac ganglion 155, superior mesenteric plexus 156, superior mesenteric ganglion 157, renal plexus 158, renal ganglion 159, inferior mesenteric plexus 160, and iliac plexus 161.

Modulation is performed to modulate metabolic rate, satiety, blood pressure, heart rate, peristalsis, insulin release, CCK release, and other gastrointestinal functions. Modulation using the system and method taught, as well as equivalent modifications and variations thereof, allows the treatment of disease including obesity, bulimia, anorexia, diabetes, hypoglycemia, hyperglycemia, irritable bowel syndrome, hypertension, hypotension, shock, gastroparesis, and other disorders. Modulation includes at least one of stimulatory and inhibitory effect on neural structures.

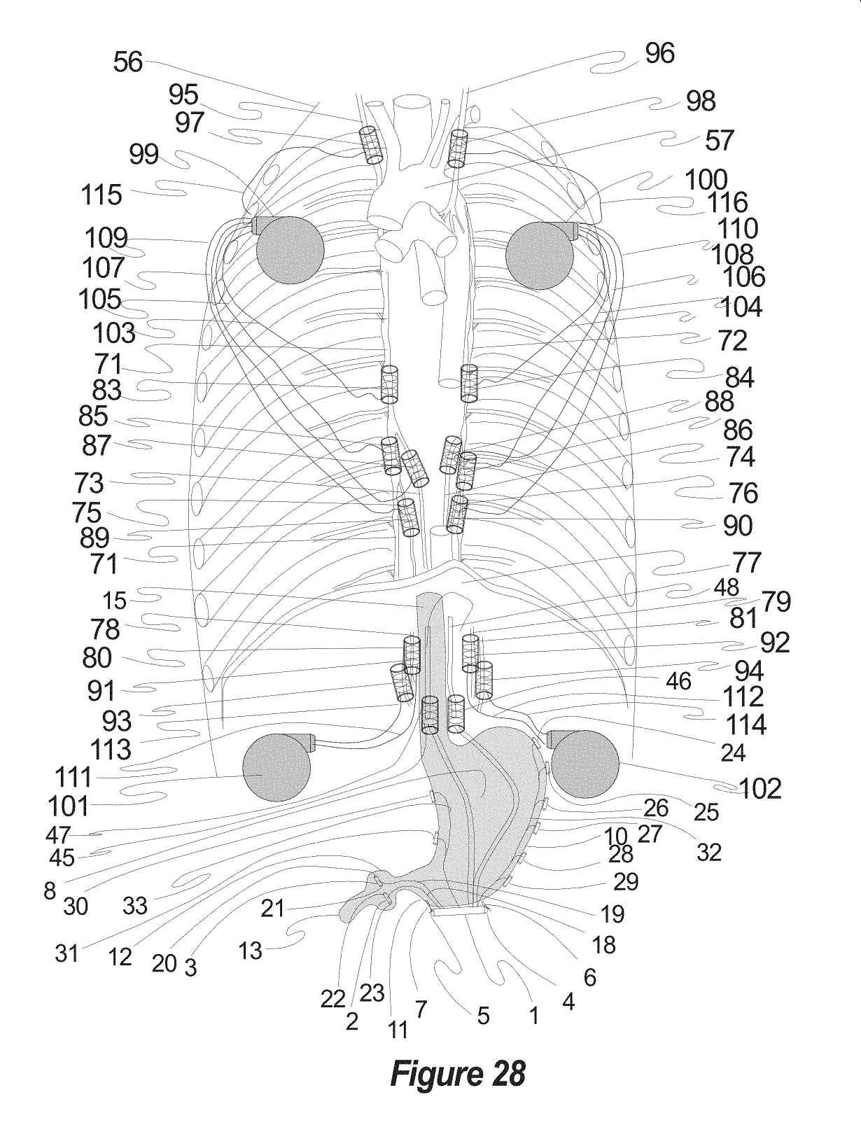

FIG. 28 shows the same invention taught in the parent case and shown in FIG. 16, with detail shown for the nerve cuff electrode implementation for the neuromodulatory interfaces. In this figure, the distal portion of the sympathetic nervous system is shown in more detail. In the parent case, modulation of the sympathetic nervous system was taught for the treatment of disease, and several nerve cuff electrode designs were presented in FIGS. 7, 8, 9, and 10 as a subset of many possible implementations of a neuromodulator or neuromodulatory interface. This FIG. 28 shows one of many potential arrangements of these components shown in the parent case; numerous other arrangements will be apparent to one skilled in the art upon reading the parent patent specification and figures.

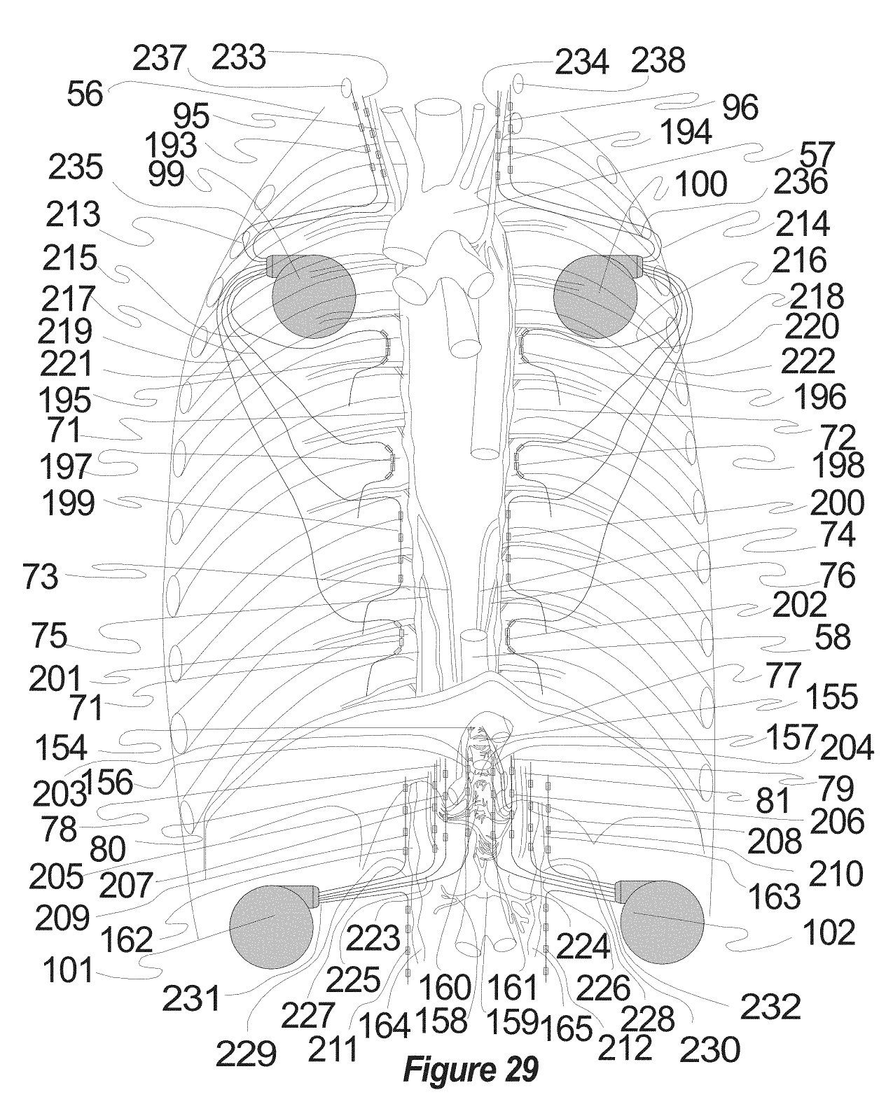

FIG. 29 shows the same invention taught in the parent case and shown in FIG. 16, with detail shown for an electrode catheter, a linear catheter-based electrode implementation for the neuromodulatory interfaces. In this figure, the distal portion of the sympathetic nervous system is shown in more detail. In the parent case, modulation of the sympathetic nervous system was taught for the treatment of disease. This FIG. 29 shows another potential arrangement of electrodes that become apparent to one skilled in the art upon reading the parent patent specification and figures.

Implantable pulse generator 99 is connected via connecting cable 213, 215, 217, 219, 221, and 235 to Right Cervical Plexus Neuromodulator Array 193, Right Intercostal Neuromodulator Array 195, Right Intercostal Neuromodulator Array 197, Right Intercostal Neuromodulator Array 199, Right Intercostal Neuromodulator Array 201, and Right Vagal Neuromodulator Array 233, respectively.

Implantable pulse generator 100 is connected via connecting cable 214, 216, 218, 220, 222, and 236 to Left Cervical Plexus Neuromodulator Array 194, Left Intercostal Neuromodulator Array 196, Left Intercostal Neuromodulator Array 198, Left Intercostal Neuromodulator Array 200, and Left Intercostal Neuromodulator Array 202, and Left Vagal Neuromodulator Array 234, respectively.

Implantable pulse generator 101 is connected via connecting cable 223, 225, 227, 229, and 231 to Right Abdominal Pam Plexus Neuromodulator Array 203, Right Abdominal Greater Splanchnic Neuromodulator Array 205, Right Abdominal Lesser Splanchnic Neuromodulator Array 207, Right Abdominal Sympathetic Trunk Neuromodulator Array 209, and Right Abdominal Sympathetic Trunk Neuromodulator Array 211, respectively

Implantable pulse generator 102 is connected via connecting cable 224, 226, 228, 230, and 232 to Left Abdominal Para Plexus Neuromodulator Array 204, Left Abdominal Greater Splanchnic Neuromodulator Array 206, Left Abdominal Lesser Splanchnic Neuromodulator Array 208, Left Abdominal Sympathetic Trunk Neuromodulator Array 210, and Left Abdominal Sympathetic Trunk Neuromodulator Array 212, respectively

Right Cervical Plexus Neuromodulator Array 193 modulates neural activity in Right Cervical Plexus 237. Right Intercostal Neuromodulator Array 195, Right Intercostal Neuromodulator Array 197, Right Intercostal Neuromodulator Array 199, and Right Intercostal Neuromodulator Array 201 each modulate neural activity in at least one of Right Sympathetic Trunk 71, Right Greater Splanchnic Nerve 73, and Right Lesser Splanchnic Nerve 75. Right Vagal Neuromodulator Array 233 modulates neural activity in Right Vagus Nerve 95.

Left Cervical Plexus Neuromodulator Array 194 modulates neural activity in Left Cervical Plexus 238. Left Intercostal Neuromodulator Array 196, Left Intercostal Neuromodulator Array 198, Left Intercostal Neuromodulator Array 200, and Left Intercostal Neuromodulator Array 202 each modulate neural activity in at least one of Left Sympathetic Trunk 72, Left Greater Splanchnic Nerve 74, and Left Lesser Splanchnic Nerve 76. Left Vagal Neuromodulator Array 234 modulates neural activity in Left Vagus Nerve 96.

Right Abdominal Para Plexus Neuromodulator Array 203 modulates at least one of Celiac Plexus 154, Celiac Ganglion 155, Superior Mesenteric Plexus 156, Superior Mesenteric Ganglion 157, Renal Plexus 158, Renal Ganglion 159, Inferior Mesenteric Plexus 160, and Iliac Plexus 161. Right Abdominal Greater Splanchnic Neuromodulator Array 205 modulates Right Subdiaphragmatic Greater Splanchnic Nerve 78. Right Abdominal Lesser Splanchnic Neuromodulator Array 207 modulates Right Subdiaphragmatic Lesser Splanchnic Nerve 80. Right Abdominal Sympathetic Trunk Neuromodulator Array 209 and Right Abdominal Sympathetic Trunk Neuromodulator Array 211 each modulate at least one of Right Lumbar Sympathetic Ganglia 162, Right Sacral Sympathetic Ganglia 164, and Right Sympathetic Trunk 71.

Left Abdominal Pam Plexus Neuromodulator Array 204 modulates at least one of Celiac Plexus 154, Celiac Ganglion 155, Superior Mesenteric Plexus 156, Superior Mesenteric Ganglion 157, Renal Plexus 158, Renal Ganglion 159, Inferior Mesenteric Plexus 160, and Iliac Plexus 161. Left Abdominal Greater Splanchnic Neuromodulator Array 206 modulates Left Subdiaphragmatic Greater Splanchnic Nerve 79. Left Abdominal Lesser Splanchnic Neuromodulator Array 208 modulates Left Subdiaphragmatic Lesser Splanchnic Nerve 81. Left Abdominal Sympathetic Trunk Neuromodulator Array 210 and Left Abdominal Sympathetic Trunk Neuromodulator Array 212 each modulate at least one of Left Lumbar Sympathetic Ganglia 163, Left Sacral Sympathetic Ganglia 165, and Left Sympathetic Trunk 72.

Elements comprising neuromodulators and neuromodulator arrays provide at least one of activating or inhibiting influence on neural activity of respective neurological target structures. Additional or fewer connecting cables and neuromodulator arrays may be employed without departing from the present invention.

These connections provided by connecting cables may facilitate communication and/or power transmission via electrical energy, ultrasound energy, optical energy, radiofrequency energy, electromagnetic energy, thermal energy, mechanical energy, chemical agent, pharmacological agent, or other signal or power means without departing from the parent or present invention.

Neuromodulator and neuromodulatory interface may be used interchangeably in this specification. Neuromodulator is a subset of modulator and modulates neural tissue.

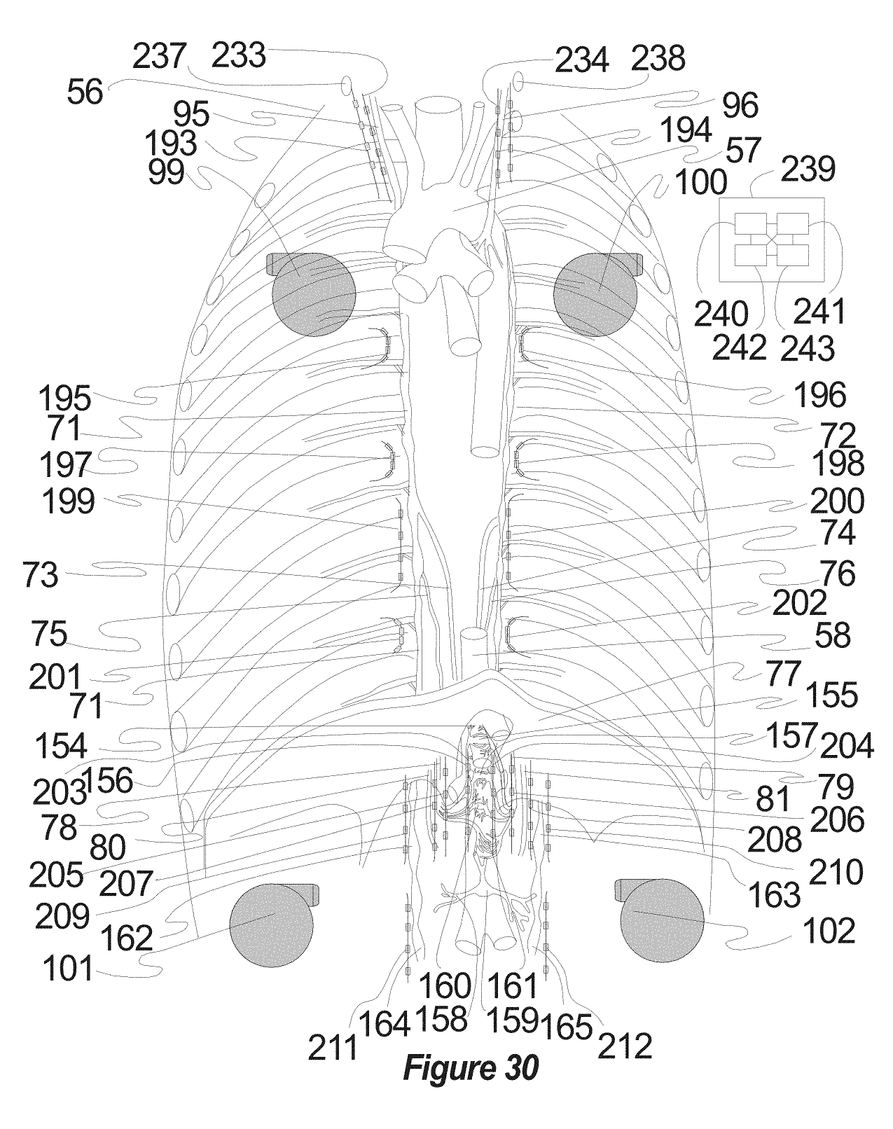

FIG. 30 shows the same invention taught in the parent case and shown in FIG. 16, with detail shown for a telemetrically powered linear catheter-based electrode implementation for the neuromodulatory interfaces. In this FIG. 30, the distal portion of the sympathetic nervous system is shown in more detail. In the parent case, modulation of the sympathetic nervous system was taught for the treatment of disease. This FIG. 30 shows the same neuromodulator configuration shown in FIG. 29, which is a potential arrangement of electrodes that becomes apparent to one skilled in the art upon reading the parent patent specification and figures. Each of the neuromodulator arrays includes a means for bidirectional transmission of information and power to and from at least one of an implantable pulse generator 99. 100, 101, and 102, and an External Transmitting and Receiving Unit 239. Each of the neuromodulator arrays includes a telemetry module, which serves as a means for bidirectional transmission of information and power to and from at least one of an implantable pulse generator 99. 100, 101, and 102 and External Transmitting and Receiving Unit 239. Each of the neuromodulator arrays includes a means for bidirectional transmission of information and power to and from at least one of an External Transmitting and Receiving Unit 239. Each of the implantable pulse generator 99. 100, 101, and 102 includes a means for bidirectional transmission of information and power to and from at least one of an External Transmitting and Receiving Unit 239.

External Transmitting and Receiving Unit 239 comprises modules including Controller 240, Memory 241, Bidirectional Transceiver 242, and User Interface 243. Additional or fewer modules may be included without departing from the present invention.

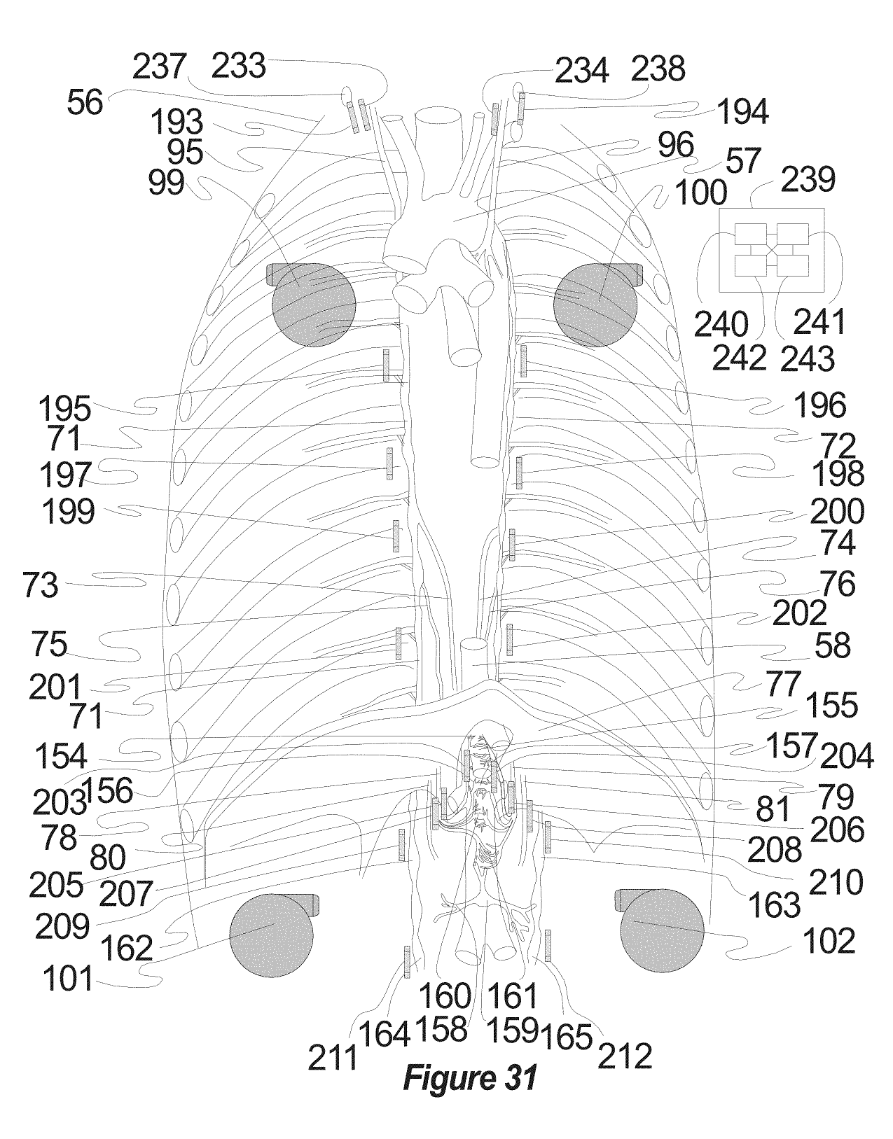

FIG. 31 shows the same invention taught in the parent case and shown in FIG. 16, with detail shown for a telemetrically powered miniature enclosure-based electrode implementation for the neuromodulatory interfaces. In one preferred embodiment, the neuromodulatory interfaces are implemented as injectable cylinders. These may have other cross-sectional shapes, including flat meshes, paddles, or grid arrays, without departing from this invention. These may have other longitudinal profiles, including rectangular, tapered, serrated, convex, biconcave, or disk shapes, without departing from this invention. In this FIG. 31, the distal portion of the sympathetic nervous system is shown in more detail. In the parent case, modulation of the sympathetic nervous system was taught for the treatment of disease. This FIG. 31 shows the same neuromodulator configuration shown in FIG. 29, which is a potential arrangement of electrodes that becomes apparent to one skilled in the art upon reading the parent patent specification and figures. Each of the neuromodulator arrays includes a means for bidirectional transmission of information and power to and from at least one of an implantable pulse generator 99. 100, 101, and 102, and an External Transmitting and Receiving Unit 239. The cylindrical enclosure-based electrode implementation for the neuromodulatory interfaces may further be injectable or implantable via laparoscopic procedure, to facilitate minimally invasive implantation.

Neuromodulatory interfaces include an energy storage element, such as capacitor, battery, or inductor, for storage of power for delivery to at least one of tissue and on-board electronic components.

External Transmitting and Receiving Unit 239 comprises modules including Controller 240, Memory 241, Bidirectional Transceiver 242, and User Interface 243. Additional or fewer modules and additional or fewer neuromodulatory interfaces may be included without departing from the present invention.

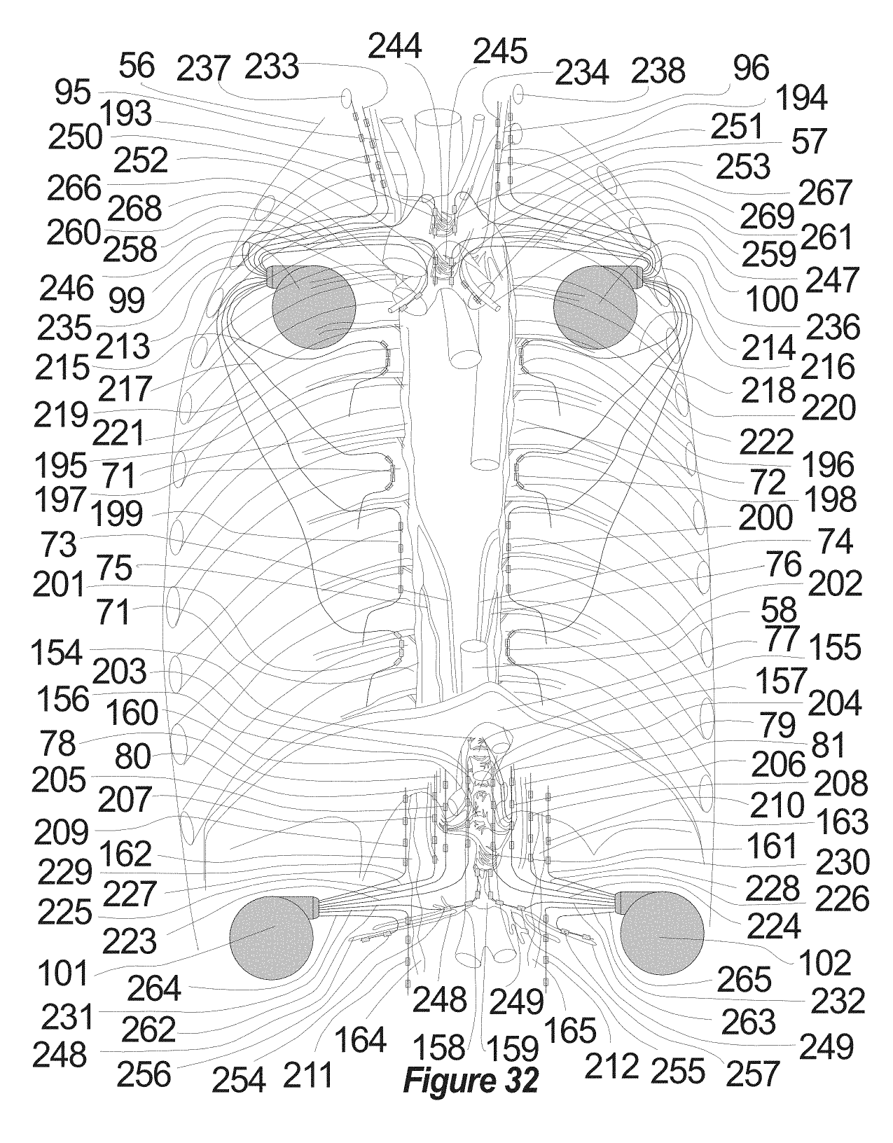

FIG. 32: shows the same invention taught in the parent case and shown in FIG. 16, with more anatomic detail shown for the autonomic nervous system and with placement of neuromodulatory interfaces for modulation of these structures.

In addition to the thoracic anatomical structures shown on FIG. 29, the superficial cardiac plexus 244, deep cardiac plexus 245, right anterior pulmonary nerve 246, and left anterior pulmonary nerve 247 are depicted in FIG. 32.

In addition to the abdominal anatomical structures shown on FIG. 29, the renal plexus 158 and renal ganglion 159 are shown with more branches, including the right renal nerve branch 248, and left renal nerve branch 249.

The activities of these structures are modulated by corresponding neuromodulatory interfaces. Any of the previously described neuromodulatory interfaces in the parent case and the present case may be positioned to modulate these neural structures. Additional or alternate designs for neuromodulatory interfaces may be employed without departing from the present or parent invention.

Implantable pulse generator 99 is connected via connecting cable 213, 215, 217, 219, 221, 235, 258, 260, and 268 to Right Cervical Plexus Neuromodulator Array 193, Right Intercostal Neuromodulator Array 195, Right Intercostal Neuromodulator Array 197, Right Intercostal Neuromodulator Array 199, Right Intercostal Neuromodulator Array 201, and Right Vagal Neuromodulator Array 233, Right Superficial Cardiac Plexus Neuromodulator Array 250, Right Deep Cardiac Plexus Neuromodulator Array 252, Right Anterior Pulmonary Nerve Neuromodulator Array 266, respectively.

Implantable pulse generator 100 is connected via connecting cable 214, 216, 218, 220, 222, 236, 259, 261, and 269 to Left Cervical Plexus Neuromodulator Array 194, Left Intercostal Neuromodulator Array 196, Left Intercostal Neuromodulator Array 198, Left Intercostal Neuromodulator Array 200, and Left Intercostal Neuromodulator Array 202, and Left Vagal Neuromodulator Array 234, Left Superficial Cardiac Plexus Neuromodulator Array 251, Left Deep Cardiac Plexus Neuromodulator Array 253, Left Anterior Pulmonary Nerve Neuromodulator Array 267, respectively.

Implantable pulse generator 101 is connected via connecting cable 223, 225, 227, 229, 231, 262, and 264 to Right Abdominal Para Plexus Neuromodulator Array 203, Right Abdominal Greater Splanchnic Neuromodulator Array 205, Right Abdominal Lesser Splanchnic Neuromodulator Array 207, Right Abdominal Sympathetic Trunk Neuromodulator Array 209, and Right Abdominal Sympathetic Trunk Neuromodulator Array 211, Right Renal Plexus Neuromodulator Array 254, and Right Renal Nerve Branch Neuromodulator Array 256, respectively.

Implantable pulse generator 102 is connected via connecting cable 224, 226, 228, 230, 232. 263, and 265 to Left Abdominal Para Plexus Neuromodulator Array 204, Left Abdominal Greater Splanchnic Neuromodulator Array 206, Left Abdominal Lesser Splanchnic Neuromodulator Array 208, Left Abdominal Sympathetic Trunk Neuromodulator Array 210, and Left Abdominal Sympathetic Trunk Neuromodulator Array 212, Left Renal Plexus Neuromodulator Array 255, and Left Renal Nerve Branch Neuromodulator Array 257, respectively

Right Cervical Plexus Neuromodulator Array 193 modulates neural activity in Right Cervical Plexus 237. Right Intercostal Neuromodulator Array 195, Right Intercostal Neuromodulator Array 197, Right Intercostal Neuromodulator Array 199, and Right Intercostal Neuromodulator Array 201 each modulate neural activity in at least one of Right Sympathetic Trunk 71, Right Greater Splanchnic Nerve 73, and Right Lesser Splanchnic Nerve 75. Right Vagal Neuromodulator Array 233 modulates neural activity in Right Vagus Nerve 95.

Right Superficial Cardiac Plexus Neuromodulator Array 250 modulates neural activity in at least one of Superficial Cardiac Plexus 244 and other structures. Right Deep Cardiac Plexus Neuromodulator Array 252 modulates neural activity in at least one of Deep Cardiac Plexus 245 and other structures. Right Anterior Pulmonary Nerve Neuromodulator Array 266 modulates neural activity in at least one of Right Anterior Pulmonary Nerve 246 and other structures.

Left Cervical Plexus Neuromodulator Array 194 modulates neural activity in Left Cervical Plexus 238. Left Intercostal Neuromodulator Array 196, Left Intercostal Neuromodulator Array 198, Left Intercostal Neuromodulator Array 200, and Left Intercostal Neuromodulator Array 202 each modulate neural activity in at least one of Left Sympathetic Trunk 72, Left Greater Splanchnic Nerve 74, and Left Lesser Splanchnic Nerve 76. Left Vagal Neuromodulator Array 234 modulates neural activity in Left Vagus Nerve 96.

Left Superficial Cardiac Plexus Neuromodulator Array 251 modulates neural activity in at least one of Superficial Cardiac Plexus 244 and other structures. Left Deep Cardiac Plexus Neuromodulator Array 253 modulates neural activity in at least one of Deep Cardiac Plexus 245 and other structures. Left Anterior Pulmonary Nerve Neuromodulator Array 267 modulates neural activity in at least one of Left Anterior Pulmonary Nerve 247 and other structures.

Right Abdominal Para Plexus Neuromodulator Array 203 modulates neural activity in at least one of Celiac Plexus 154, Celiac Ganglion 155, Superior Mesenteric Plexus 156, Superior Mesenteric Ganglion 157, Renal Plexus 158, Renal Ganglion 159, Inferior Mesenteric Plexus 160, and Iliac Plexus 161. Right Abdominal Greater Splanchnic Neuromodulator Array 205 modulates neural activity in Right Subdiaphragmatic Greater Splanchnic Nerve 78. Right Abdominal Lesser Splanchnic Neuromodulator Array 207 modulates neural activity in Right Subdiaphragmatic Lesser Splanchnic Nerve 80. Right Abdominal Sympathetic Trunk Neuromodulator Array 209 and Right Abdominal Sympathetic Trunk Neuromodulator Array 211 each modulate neural activity in at least one of Right Lumbar Sympathetic Ganglia 162, Right Sacral Sympathetic Ganglia 164, and Right Sympathetic Trunk 71.

Right Renal Plexus Neuromodulator Array 254 modulates neural activity in at least one of Right Renal Nerve Branch 248, Renal Plexus 158, Renal Ganglion 159, and other structures. Right Renal Nerve Branch Neuromodulator Array 256 modulates neural activity in at least one of Right Renal Nerve Branch 248, Renal Plexus 158, Renal Ganglion 159, and other structures.

Left Abdominal Para Plexus Neuromodulator Array 204 modulates neural activity in at least one of Celiac Plexus 154, Celiac Ganglion 155, Superior Mesenteric Plexus 156, Superior Mesenteric Ganglion 157, Renal Plexus 158, Renal Ganglion 159, Inferior Mesenteric Plexus 160, and Iliac Plexus 161. Left Abdominal Greater Splanchnic Neuromodulator Array 206 modulates neural activity in Left Subdiaphragmatic Greater Splanchnic Nerve 79. Left Abdominal Lesser Splanchnic Neuromodulator Array 208 modulates neural activity in Left Subdiaphragmatic Lesser Splanchnic Nerve 81. Left Abdominal Sympathetic Trunk Neuromodulator Array 210 and Left Abdominal Sympathetic Trunk Neuromodulator Array 212 each modulate neural activity in at least one of Left Lumbar Sympathetic Ganglia 163, Left Sacral Sympathetic Ganglia 165, and Left Sympathetic Trunk 72.

Left Renal Plexus Neuromodulator Array 255 modulates neural activity in at least one of Left Renal Nerve Branch 249, Renal Plexus 158, Renal Ganglion 159, and other structures. Left Renal Nerve Branch Neuromodulator Array 257 modulates neural activity in at least one of Left Renal Nerve Branch 249, Renal Plexus 158, Renal Ganglion 159, and other structures.

Elements comprising neuromodulators and neuromodulator arrays provide at least one of activating or inhibiting influence on neural activity of respective neurological target structures. Additional or fewer connecting cables and neuromodulator arrays may be employed without departing from the present invention.

These connections provided by connecting cables may facilitate communication and/or power transmission via electrical energy, ultrasound energy, optical energy, radiofrequency energy, electromagnetic energy, thermal energy, mechanical energy, chemical agent, pharmacological agent, or other signal or power means without departing from the parent or present invention.

Neuromodulators and neuromodulatory interfaces may be used interchangeably in this specification.

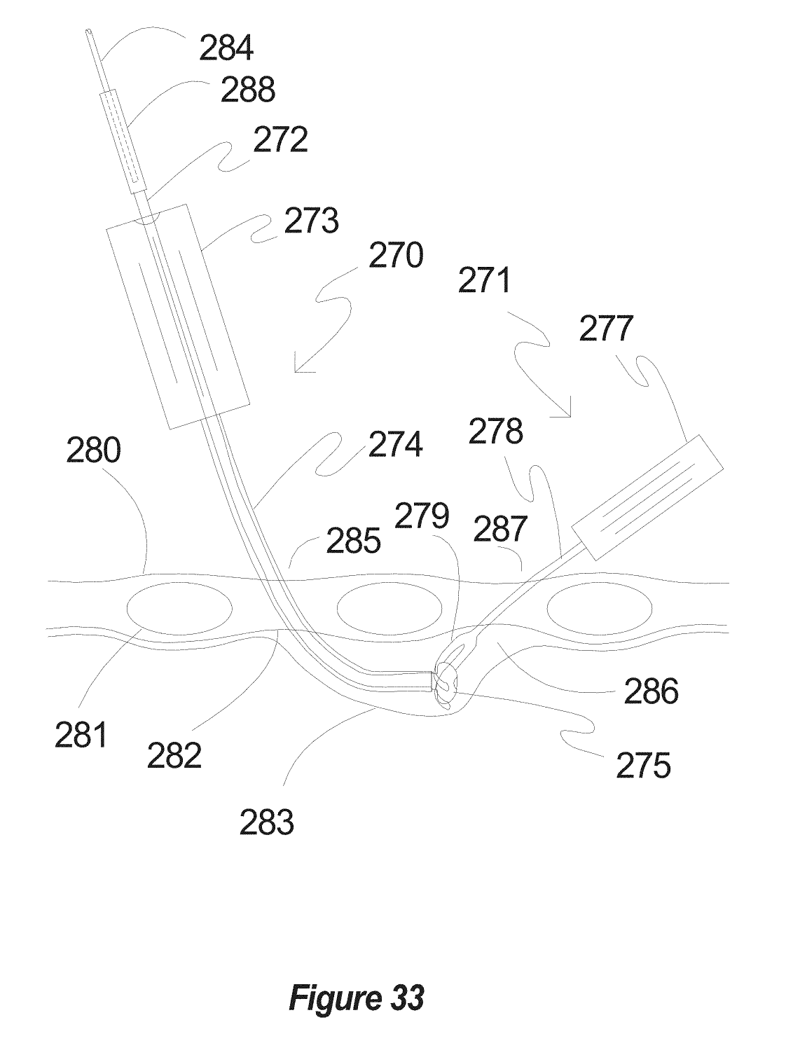

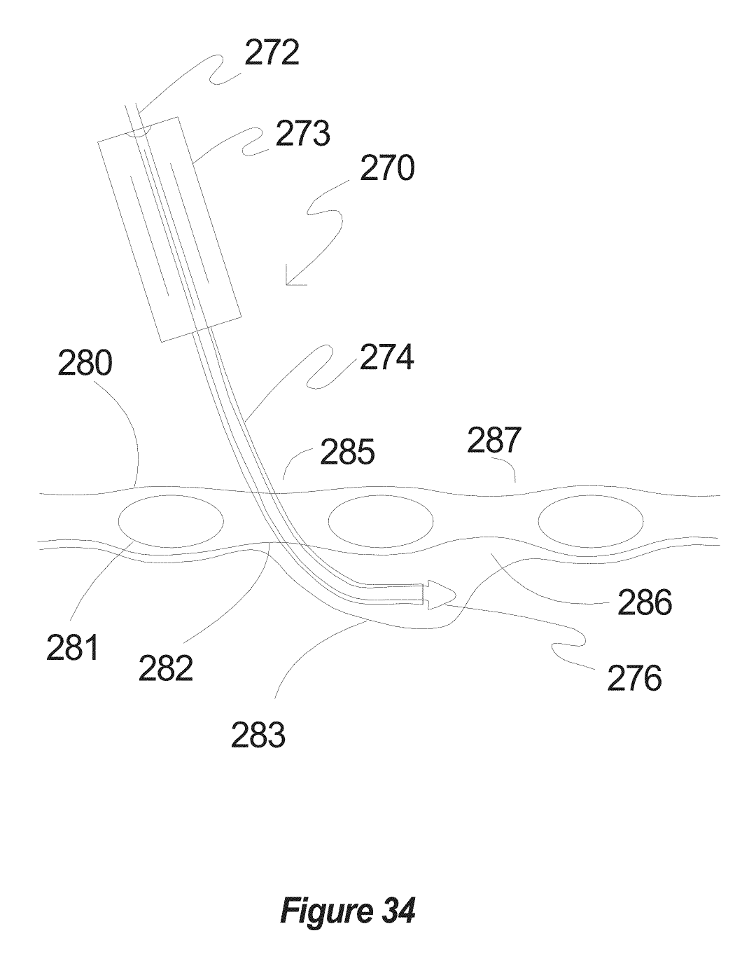

FIGS. 33 and 34: show the catheter insertion trocar 270 during intraoperative use for placement of neuromodulatory interface array catheter 284. Surgeon or assistant makes incision in skin 280, at entry point 285 in the cervical, thoracic, lumbar, or sacral region. FIGS. 33 and 34 depict a skin incision at an entry point 285 which is shown in a representative site in the thoracic or lumbar region. Surgeon grasps catheter insertion trocar handle 273 and applies force which is transmitted through catheter insertion trocar shaft 274 to advance catheter insertion trocar bulb tip 275 through skin 280 and parietal pleura 282 into the potential space labeled pleural space 286 which is expanded by this procedure. Entry point 285 and exit point 287 are shown adjacent to but not directly overlying any of rib 281; however, either or both of entry point 285 and exit point 287 may overly any of rib 281, in which case tunneling under skin or through rib may be performed.

Care is taken to avoid perforating visceral pleura 283. Skin incision is made at entry point 285 through the majority of the thickness of skin 280 close to parietal pleura 282 to assist in minimizing the amount of force required to enter pleural space 286, thereby minimizing the velocity and acceleration of catheter insertion trocar bulb tip 275 during this procedure and reducing the risk of perforation of visceral pleura 283. A novelty of the present invention, shown in FIG. 33, is the shape of catheter insertion trocar bulb tip 275, which is curved to further reduce the risk of perforation of visceral pleura 283.

Catheter insertion retriever 271 is inserted through an incision in skin 280 at the site of exit point 287. Surgeon or assistant grasps catheter insertion retriever handle 277, and with catheter insertion retriever shaft 286 penetrating skin 280, positions catheter insertion retriever grasper 279 to grasp catheter insertion trocar bulb tip 275 and to pull or guide attached catheter 272 through incision in skin 280 at exit point 287.

As shown in FIG. 33, catheter insertion trocar bulb tip 275 may be part of catheter 272. Tensile and shear force applied through catheter insertion retriever grasper 279 is applied to pull and guide, respectively, catheter 272 in its advancement through pleural space 286 and through parietal pleura 282 and skin 280 at the site of exit point 287. Catheter attachment means 288 at the trailing end of catheter 272 enables neuromodulatory interface array catheter 284 to be pulled through skin 280 and parietal pleura 282 at entry point 285, through pleural space 286, and through parietal pleura 282 and skin 280 at exit point 287. Depending on the design, catheter insertion trocar 270 may be withdrawn prior to attachment of catheter 272 to neuromodulatory interface array catheter 284. Alternately, if said catheter attachment means 288 is sufficiently small relative to the internal diameter of catheter insertion trocar shaft 274, catheter insertion trocar 270 may be withdrawn after attachment of catheter 272 to neuromodulatory interface array catheter 284 and advancement of neuromodulatory interface array catheter 284 through skin 280 at exit point 287.

FIG. 34 depicts a pointed design which facilitates advancement of catheter insertion trocar 270 into pleural space 286 and back through parietal pleura 282 and skin 280 at the site of exit point 287. As shown in this figure, pointed tip 276 is attached to or part of catheter 272. Alternatively, pointed tip 276 may be attached to or part of catheter insertion trocar shaft 274, without departing from the present invention.

In both FIG. 33 and FIG. 34, catheter 272 may serve as a guide to facilitate advancement of neuromodulatory interface array catheter 284 into position, as described above. Alternately, to save time and to reduce procedural complexity, catheter 272 may be replaced with neuromodulatory interface array catheter 284, without departing form the present invention. In this latter configuration, neuromodulatory interface array catheter 284 is advanced into position by catheter insertion trocar 270 in either of the two methods described and shown in FIG. 33 and FIG. 34.

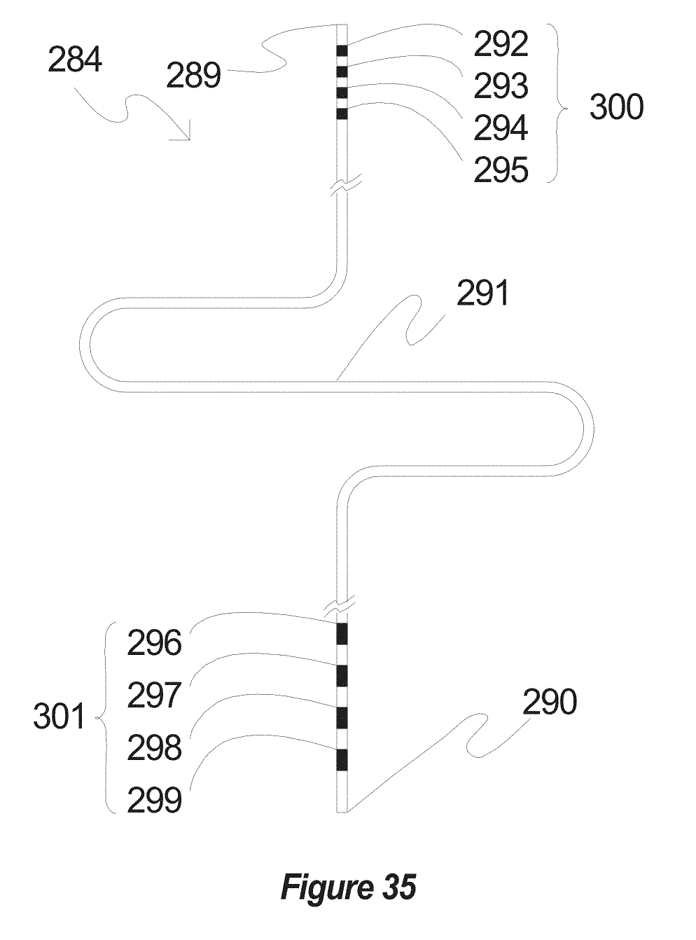

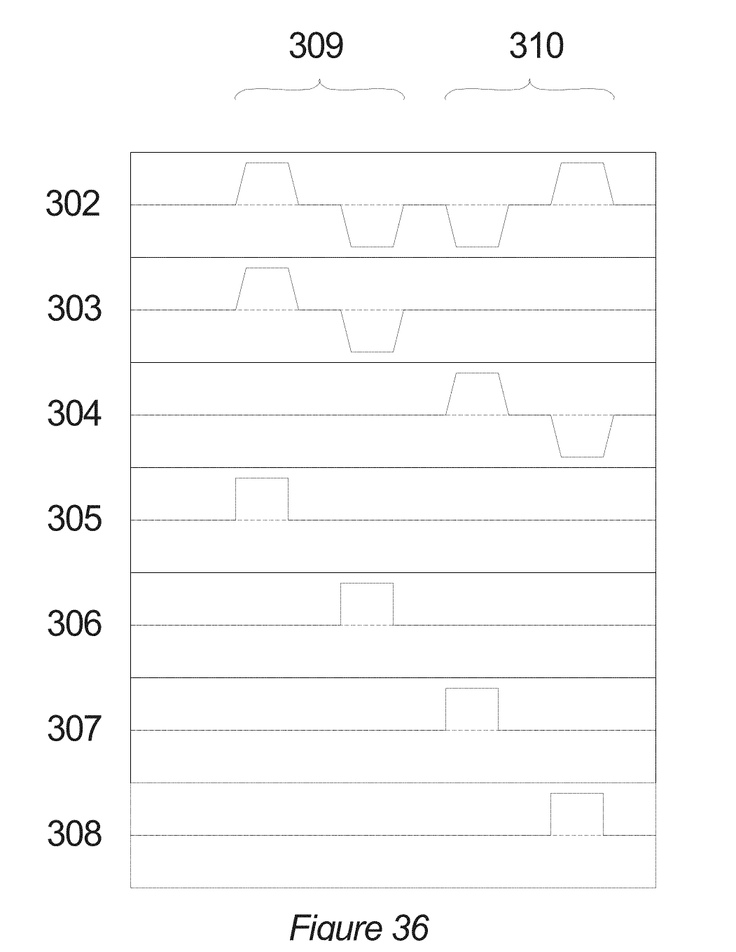

FIG. 35 shows the neuromodulatory interface array catheter 284 which represent another implementation of the neuromodulatory interface 34 taught in the parent case and shown in multiple forms in FIG. 16. In this embodiment, at least one neuromodulatory interface 34 is implemented as a single or plurality of neuromodulatory interface array catheter 284.

Neuromodulatory interface array catheter 284 comprises a connector contact array 300 located near connector end 289, a neuromodulatory interface array 301 located near neuromodulatory interface end 290, and catheter body 291, which provides mechanical connection and signal transmission between connector contact array 300 and neuromodulatory interface array 301. Said signal transmission may be in the form of electrical fields or energy, electrical voltage, electrical current, optical energy, magnetic fields or energy, electromagnetic fields or energy, mechanical force or energy, vibratory force or energy, chemical agent or activation, pharmacological agent or activation, or other signal transmission means.

Neuromodulatory interface array 301 is comprised of at least one of neuromodulatory interface 296, 297, 298, and 299. Additional or fewer numbers of neuromodulatory interface may comprise neuromodulatory interface array 301 without departing from the present invention. Neuromodulator interface 296, 297, 298, 299 modulate activity of neural structures using at least one of electrical fields or energy, electrical voltage, electrical current, optical energy, magnetic fields or energy, electromagnetic fields or energy, mechanical force or energy, vibratory force or energy, chemical agent or activation, pharmacological agent or activation, or other neural modulation means.

Connector contact array 300 is comprised of at least one of connector element 292, 293, 294, and 295. Additional or fewer numbers of connector element may comprise connector contact array 300 without departing from the present invention.

FIG. 36 shows the effects of modulation of the autonomic nervous system, including periods of sympathetic modulation 309 and parasympathetic modulation 310. Sympathetic modulation 309 may be performed by stimulating or inhibiting activity in a portion of the sympathetic nervous system. Parasympathetic modulation 310 may be performed by stimulating or inhibiting activity in a portion of the parasympathetic nervous system.

Tracings showing the level of sympathetic stimulation 305 and sympathetic inhibition 306 are shown. During the time window in which sympathetic stimulation 305 is active, the sympathetic index 303 is seen to be increased and the autonomic index 302 is increased. During the time window in which sympathetic inhibition 306 is active, the sympathetic index 303 is seen to be decreased and the autonomic index 302 is decreased.

Tracings showing the level of parasympathetic stimulation 307 and parasympathetic inhibition 308 are shown. During the time window in which parasympathetic stimulation 307 is active, the parasympathetic index 304 is seen to be increased and the autonomic index 302 is decreased. During the time window in which parasympathetic inhibition 308 is active, the parasympathetic index 304 is seen to be decreased and the autonomic index 302 is increased.

Sympathetic and parasympathetic inhibition is accomplished by blockage of neural fibers. This is be performed using high frequency stimulation, with a best mode involving biphasic charge balanced waveforms delivered at frequencies over 100 Hz, though significantly higher as well as lower frequencies may be employed without departing form the present invention.

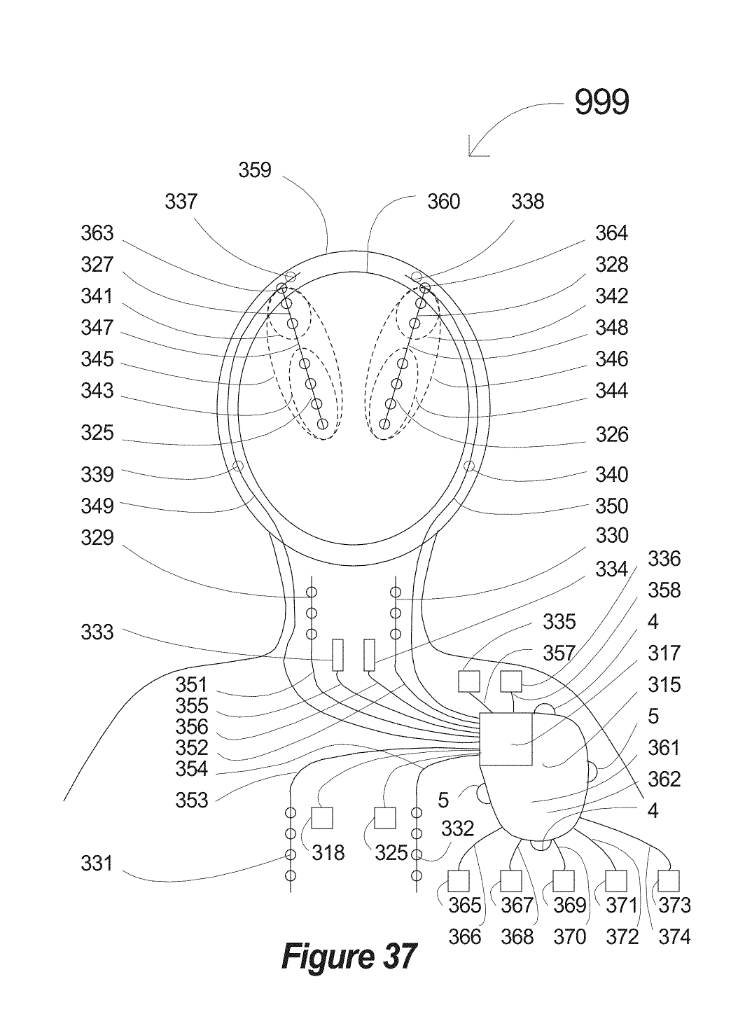

E. Intracranial—Subclavicular components. FIG. 37 Shows a closed-loop stimulator circuit placed in a subclavicular pocket with intracranial and peripheral components.

FIG. 37 is a schematic diagram of one embodiment of the neurological control system 999 of the present invention shown implanted in a human patient. The neurological control system 999 could be external or implanted as shown. A single or plurality of neurological control system 999, including bilateral application, may be used. Each neurological control system 999 includes a stimulating and recording unit 315 and one or more intracranial and extracranial components described below. As described in this illustrative embodiment, the intracranial components preferably include a neuromodulator array 316. These may be implemented as stimulating electrodes or as other elements designed to impart signals to neural structures and thereby modulate neural activity, including optical, ultrasound, electromagnetic sources as well as pharmacological or chemical emitters, or other means to alter neural activity. However, it should become apparent to those of ordinary skill in the relevant art after reading the present disclosure that the stimulating electrodes may also be extracranial; that is, attached to a peripheral nerve or autonomic neural structure in addition to or in place of being located within the cranium. As shown in FIG. 37, stimulating and recording unit 315 of neurological control system 999 is preferably implanted in a subclavicular pocket. Alternately it may be implanted in a pericranial location, such as being recessed in the calvarium. Header 317 facilitates signal communication between stimulating and recording unit 315 and other components of neurological control system 999, such as neuromodulator array 316 and other sensors, modulators, communications modules, and other components. Some or all of the connections facilitated by header 317 may alternately be implemented using wireless technology.

As one skilled in the relevant art would find apparent from the following description, the configuration illustrated in FIG. 37 is just one example of the present invention. Many other configurations are contemplated. For example, in alternative embodiments of the present invention, the stimulating and recording unit 315 is implanted ipsilateral or bilateral to particular intracranial or extracranial components. It should also be understood that the stimulating and recording unit 315 can receive ipsilateral, contralateral or bilateral inputs from sensors and deliver ipsilateral, contralateral, or bilateral outputs to a single or a plurality of intracranial or extracranial neuromodulator arrays 316, including stimulating and recording electrode arrays. Preferably, these inputs are direct or preamplified signals from at least one of sensor array 323, including neural sensor array 318, physiological sensor array 319, EMG sensor array 320, metabolic sensor array 321, alimentation sensor array 322, or other sensor array. Physiological sensor array 319 includes single and multiple modality sensor arrays, including but not limited to accelerometer array, acoustic transducer array, gastrointestinal pressure sensor array, gastrointestinal strain sensor array, gastrointestinal stress sensor array, temperature sensor array, glucose sensor array, heart rate sensor array, blood pressure sensor array, respiratory rate sensor array, respiratory pressure sensor array, respiratory acoustic sensor array, patient input sensor array, or other sensor array. Neural sensor array 318 includes any sensor which generates a signal representative of neural activity, including but not limited to peripheral nerve electrode array, intracranial recording electrode array, other electrode array, neuromodulator array, or other neural sensing device. The signals input from these sensors will be referred to herein as “sensory input modalities” 324. The outputs include but are not limited to one or more signals, such as stimulating current signals or stimulating voltage signals or stimulating optical signals, to neuromodulator array 316.

Neuromodulator array 316 includes but is not limited to neuromodulator array 318, 319, 320, 321, 322, 323, modulator 2, 3, 24, 25, 26, 27, 28, 29, 30, 31, neuromodulatory interface 34, nerve cuff 36, longitudinal electrode array 38, regeneration electrode array 44, vagus nerve interface 45, sympathetic nerve interface 46, epineural electrode 49, 50, 51, sympathetic trunk neuromodulatory interface 83, 84, 85, 86, thoracic splanchnic neuromodulatory interface 87, 88, 89, 90, abdominal splanchnic neuromodulatory interface 91, 92, 93, 94, vagus neuromodulatory interface 97, 98, epineural cuff electrode neuromodulatory interface 117, longitudinal electrode neuromodulatory interface 118, 119, regeneration tube neuromodulatory interface 120, anterior central spinal neuromodulatory interface 143, anterior right lateral spinal neuromodulatory interface 144, anterior left lateral spinal neuromodulatory interface 145, posterior central spinal neuromodulatory interface 146, posterior right lateral spinal neuromodulatory interface 147, posterior left lateral spinal neuromodulatory interface 148, right lateral spinal neuromodulatory interface 149, left lateral spinal neuromodulatory interface 150, intermediolateral nucleus neuromodulatory interface 152, abdominal splanchnic neuromodulatory interface 170, 171, neuromodulator array 174, 175, abdominal splanchnic neuromodulatory interface 178, 179, 180, 181, 182, 193, 184, 185, 186, right cervical plexus neuromodulatory array 193, left cervical plexus neuromodulatory array 194, right intercostal neuromodulatory array 195, 197, 199, 201, left intercostal neuromodulatory array 196, 198, 200, 202, right abdominal para plexus neuromodulatory array 203, left abdominal para plexus neuromodulatory array 204, right abdominal superior splanchnic neuromodulatory array 205, left abdominal superior splanchnic neuromodulatory array 206, right abdominal inferior splanchnic neuromodulatory array 207, left abdominal inferior splanchnic neuromodulatory array 208, right abdominal sympathetic trunk neuromodulatory array 209, 211, left abdominal sympathetic trunk neuromodulatory array 210, 212, right vagal neuromodulator array 233, left vagal neuromodulator array 234, right superficial cardiac plexus neuromodulator array 250, left superficial cardiac plexus neuromodulator array 251, right deep cardiac plexus neuromodulator array 252, left deep cardiac plexus neuromodulator array 253, right renal plexus neuromodulator array 254, left renal plexus neuromodulator array 255, right renal nerve branch neuromodulator array 256, left renal nerve branch neuromodulator array 257, right anterior pulmonary nerve neuromodulator array 266, left anterior pulmonary nerve neuromodulator array 267, neuromodulatory interface 296, 297, 298, 299, neuromodulatory interface array 301, neuromodulator array 316, 325, 326, 327, 328, 329, 330, 331, 332, and other apparatus or methods which modulate neural activity. A single or plurality of elements of neuromodulator array 316 may also be used as elements of a sensor array instead of or in addition to their function in modulating neural activity.

In the embodiment illustrated in FIG. 37, neurological control system 999 is shown to receive bilateral sensory inputs and to deliver outputs through bilateral instances of neuromodulator array 316. In the illustrative embodiment, neurological control system 999 also receives sensory inputs from neuromodulator array 316 and sensory input modalities 324, including neural sensor array 318, physiological sensor array 319, EMG sensor array 320, metabolic sensor array 321, alimentation sensor array 322, and other sensors arrays 323. Neural sensor array 321 comprises all neuromodulators 316 (including neuromodulator arrays 325, 326, 327, 328, 329, 330, 331, and 332) and neural sensors and electrodes, including EEG electrodes 337, 338, 339, and 340. Physiological Sensor Array 319 comprises physiological sensor array 333, 334, and 335, physiological sensor array 333, 334, and 335 are connected to stimulating and recording circuit 315 via physiological sensor array connecting cable 355, 356, and 357, respectively.

Physiological sensor array 319 senses at least one of any physiological parameters comprising temperature, hear rate, heart rate variability, any cardiac parameter, blood pressure, respiratory rate, respiratory function parameters and pressures, metabolic rate, respiratory quotient, glucose level, insulin level, organ perfusion, or other physiological parameter. Additional or fewer sensors and/or neuromodulators may be used without departing from the present invention.

Superficial intracranial electrode array 341 and 342 modulate and sense activity from superficial regions of the nervous system, including the cortex, subdural space, epidural space, calvaral space, subgaleal space, subcutaneous space and/or scalp region. Deep intracranial electrode array 343 and 344 modulate and sense activity from deep brain regions, including but not limited to subcortical nuclei and white matter tracts, brainstem structures, and medial and lateral and other components of the hypothalamus and all satiety centers.

Neural sensor array 318 generates neural signals representative of neural activity, including but not limited to signals from cortical, white matter, and deep brain nuclear signals. Neural activity to be sensed and neural activity to be modulated includes but is not limited to that found in the sympathetic nervous system, parasympathetic nervous system, autonomic nervous system, baroreceptor neural circuit components, primary motor cortex, premotor cortex, supplementary motor cortex, other motor cortical regions, somatosensory cortex, other sensory cortical regions, Broca's area, Wernicke's area, other cortical regions, white matter tracts associated with these cortical areas, other white matter tracts, the globus pallidus internal segment (GPi, GPi,e, GPi,e), the globus pallidus external segment, the caudate, the putamen, locus ceruleus, and other cortical and subcortical areas, ventral medial Vim thalamic nucleus, other portion of the thalamus, subthalamic nucleus (STN), caudate, putamen, other basal ganglia components, cingulate gyrus, other subcortical nuclei, nucleus locus ceruleus, pedunculopontine nuclei of the reticular formation, red nucleus, substantia nigra, other brainstem structure, cerebellum, internal capsule, external capsule, corticospinal tract, pyramidal tract, ansa lenticularis, other central nervous system structure, other peripheral nervous system structure, other intracranial region, other extracranial region, other neural structure, sensory organs, muscle tissue, or other non-neural structure.

This is one embodiment. Numerous permutations of electrode stimulation site configuration may be employed, including more or fewer electrodes in each of these said regions, without departing from the present invention. Electrodes may be implanted within or adjacent to other regions in addition to or instead of those listed above without departing from the present invention.

As one of ordinary skill in the relevant art will find apparent, the present invention may include additional or different types of sensors that sense neural responses for the type and particular patient. Such sensors generate sensed signals that may be conditioned to generate conditioned signals, as described below. Examples of the placement of these electrodes is described above with reference to the embodiment illustrated in these figures. Many others are contemplated by the present invention.

Neural sensor array 318 is connected to recording and stimulating circuit 315 with neural sensor array connecting cable 375. In one embodiment, neural sensor array 318 comprises, at least one of neuromodulatory interface 34, nerve cuff 36, longitudinal electrode array 38, regeneration electrode array 44, vagus nerve interface 45, sympathetic nerve interface 46, epineural electrode 49, 50, and 51, sympathetic trunk neuromodulatory interface 83, 84, 85, and 86, thoracic splanchnic neuromodulatory interface 87, 88, 89, and 90, abdominal splanchnic neuromodulatory interface 91, 92, 93, and 94, vagus neuromodulatory interface 97 and 98, epineural cuff electrode neuromodulatory interface 117, longitudinal electrode neuromodulatory interface 118, longitudinal electrode regeneration port neuromodulatory interface 119, regeneration tube neuromodulatory interface 120, and any other potential component comprising neuromodulator array 316, which is described above. A single or multiplicity of peripheral nerve interface 380, comprising vagus neuromodulatory interface 97 and 98, vagus nerve interface 45, sympathetic nerve interface 46, or other neural interface may be located in the cervical region, thoracic region, lumbar region, sacral region, abdominal region, pelvic region, the head, cranial nerves, neck, torso, upper extremities, and lower extremities, without departing from the present invention. Peripheral nerve interface 380, when located in the neck region, can interface with the vagus nerve, sympathetic ganglia, spinal accessory nerve, or nerve arising from cervical roots.

In one embodiment, peripheral nerve interface 380 are each comprised of three epineural platinum-iridium ring electrodes, each in with an internal diameter approximately 30% larger than that of the epineurium, longitudinally spaced along the nerve. Electrodes of differing dimensions and geometries and constructed from different materials may alternatively be used without departing from the present invention. Alternative electrode configurations include but are not limited to epineural, intrafascicular, or other intraneural electrodes; and materials include but are not limited to platinum, gold, stainless steel, carbon, and other element or alloy.

As will become apparent from the following description, signals representing various sensory input modalities 324 from sensor arrays 323 may provide valuable feedback information.

It should be understood that this depiction is for simplicity only, and that any combination of ipsilateral, contralateral or bilateral combination of each of the multiple sensory input modalities and multiple stimulation output channels may be employed. In addition, neurological control system 999 may be a single device, multiple communicating devices, or multiple independent devices. Accordingly, these and other configurations are considered to be within the scope of the present invention. It is anticipated that neurological control system 999, if implemented as distinct units, would likely be implanted in separate procedures (soon after clinical introduction) to minimize the likelihood of drastic neurological complications.

In the exemplary embodiment illustrated in FIG. 37, intracranial components 345 and 346 include intracranial catheter 347 and 348, one preferred embodiment of which comprise a plurality of intracranial stimulating and recording electrodes. Superficial intracranial electrode array 341 and 342 may, of course, have more or fewer electrodes than that depicted in FIG. 37. These intracranial stimulating electrodes may be used to provide stimulation to a predetermined nervous system component. The electrical stimulation provided by the intracranial stimulating electrodes may be excitatory or inhibitory, and this may vary in a manner which is preprogrammed, varied in real-time, computed in advance using a predictive algorithm, or determined using another technique now or latter developed.

Intracranial catheters 347 and 348 include neuromodulator arrays 325, 326, 327, and 328, which may comprise intracranial recording electrodes and/or intracranial stimulating electrodes. In accordance with one embodiment of the present invention, intracranial recording electrodes are used to record cortical activity as a measure of response to treatment and as a predictor of impeding treatment magnitude requirements. In the illustrative embodiment, neuromodulator arrays 327 and 328, which may be implemented as superficial intracranial electrode array 341 and 342 are depicted in a location superficial to neuromodulator arrays 325 and 326, which may be implemented as deep intracranial electrode arrays 343 and 344.

In the illustrative embodiment, intracranial catheters 347 and 348 are provided to mechanically support and facilitate communication of electrical, optical, or other signal and/or power modality between intracranial and extracranial structures. In this embodiment, intracranial catheters 347 and 348 contain one or more wires, optical fibers, telemetry links or other means facilitating connecting stimulating and recording circuit 315 to the intracranial components 345 and 346, including but not limited to neuromodulator array 316, which may comprise intracranial stimulating electrodes, intracranial recording electrodes, as well as extracranial stimulating electrodes and extracranial recording electrodes, and other sensors and modulators. The wires contained within intracranial catheters 347 and 348 transmit neuromodulation signal (NMS) 998 or stimulating electrode output signal (SEOS) to superficial intracranial electrode arrays 341 and 342 and to deep intracranial electrode arrays 343 and 344. Wires are understood to also include other communications medium, comprising optical fibers, ultrasound conduits, wireless telemetry modules, and the like. Such wires additionally transmit stimulating electrode input signal (SEIS) and recording electrode input signal (REIS), to and from superficial intracranial electrode arrays 341 and 342 and to and from deep intracranial electrode arrays 343 and 344. Other recording and stimulating or modulating modalities may be used in addition to or instead of electrode arrays without departing from the present invention.

Stimulating and recording circuit 315 is protected within circuit enclosure 361. Circuit enclosure 361 and contained components, including stimulating and recording circuit 315 comprise stimulating and recording unit 362. It should be understood that more or fewer of either type of electrode as well as additional electrode types and locations may be incorporated or substituted without departing from the spirit of the present invention. Furthermore, stimulating and recording circuit 315 can be placed extra cranially in a subclavian pocket as shown in FIG. 37, or it may be placed in other extracranial, intracranial, or nonimplanted locations.

Connecting cable 349 and 350 generally provide electrical, optical, chemical or other signal connection between intracranial or intracranial locations. A set of electrical wires is one mans which provides the for electrical communication between the intracranial and extracranial components; however, it should be understood that alternate systems and techniques such as radiofrequency links, optical (including infrared) links with transcranial optical windows, magnetic links, and electrical links using the body components as conductors, may be used without departing from the present invention. Specifically, in the illustrative embodiment, connecting cable 349 and 350 provide electrical connection between intracranial components 345 and 346 and stimulating and recording circuit 315. In embodiments wherein stimulating and recording circuit 315 has an intracranial location, connecting cable 349 and 350 would likely be entirely intracranial. Alternatively, connecting in embodiments wherein stimulating and recording circuit 315 is implanted under scalp 359 or within or attached to calvarium 360, connecting cable 349 and 350 may be confined entirely to subcutaneous region under the scalp 359.

A catheter anchor 363 and 364 provide mechanical connection between intracranial catheter 347 and 348 and calvarium 360. Catheter anchor 363 and 364 are preferably deep to the overlying scalp 359. Such a subcutaneous connecting cable 349 and 350 provides connection between stimulating and recording circuit 26 and at least one of superficial intracranial electrode array 341 and 342, deep intracranial electrode array 343 and 344, other neuromodulator array 316, neural sensor array 318, physiological sensor array 319, metabolic sensor array 321, or other sensor array 323. Connecting cable 349 and 350 may also connect any other sensors, including but not limited to any of sensory input modalities 324, or other stimulating electrodes, neuromodulators, medication dispensers, or actuators with stimulating and recording circuit 315.

Sensory feedback is provided to stimulating and recording circuit 315 from a multiplicity of sensors, collectively referred to as sensory input modalities 324. Neural sensor array 318 comprises superficial intracranial electrode array 341 and 342, deep intracranial electrode array 343 and 344, and other intracranial and extracranial recording electrode arrays and other neural sensors and neuromodulators. Additional sensors, some of which are located extracranially in the embodiment, comprise the remainder of sensory input modalities 324. Sensory input modalities 324 provide information to stimulating and recording circuit 315. As will be described in greater detail below, such information is processed by stimulating and recording circuit 315 to deduce the disease state and progression and its response to therapy. Disease state comprises qualities, parameters, or metrics related to any disease, disorder, or condition mentioned or related to those mentioned in the present invention or any materials incorporated by reference. For example, disease state comprises metabolic state, cardiovascular parameters, respiratory parameters, affect qualities or parameters, psychosis qualities or parameters, insulin and glucose levels or parameters, irritable bowel syndrome qualities or parameters, or any quality or metric related to a disease, disorder, condition, neurological, psychiatric, or physiological state.

In one embodiment of the invention, physiological sensor array 319 comprises an acoustic transducer array 336 to monitor any number of vibratory characteristics such as high frequency head or body vibration, muscle vibration, speech production, blood flow, air flow, and/or other physiological parameter. Acoustic transducer array 336 comprises at least one of an acoustic sensor or an acoustic transducer and is connected to stimulating and recording circuit 315 with acoustic transducer array connecting cable 358.

In one embodiment of the invention, physiological sensor array 319 comprises temperature sensor array 365 to monitor local temperature, body temperature, or ambient temperature. Temperature sensor array 365 is connected to stimulating and recording circuit 315 with temperature sensor array connecting cable 366.

In one embodiment of the invention, physiological sensor array 319 comprises respiratory sensor array 367 to monitor at least one of pulmonary pleura pressure, inter-bronchial pressure, inter-alveolar pressure, transpleural pressure, transbronchial pressure, thransthoracic pressure, other pressure related to pulmonary or respiratory function, bronchial air flow, alveolar airflow, tracheal airflow, or other airflow or blood flow related to pulmonary or respiratory function. Respiratory sensor 367 may be implemented as at least one of a pressure sensor, flow sensor, Doppler transceiver and/or sensor, acoustic sensor and/or transducer, electrical impedance sensor and/or transducer, mechanical impedance sensor and/or transducer, or other sensor or transducer. Respiratory sensor array 367 is connected to stimulating and recording circuit 315 with respiratory sensor array connecting cable 368.

In one embodiment of the invention, physiological sensor array 319 comprises pressure sensor array 369 to monitor a pressure related to function of at least one of pulmonary function, respiratory function, cardiac function, cardiovascular function, vascular function, gastrointestinal function, alimentary function, gastric function, pyloric function, duodenum function, jejunum function, ileum function, small intestinal function, large intestine function, cecum function, sigmoid function, rectum function, bladder function, ovulatory function, ejaculatory function, other pressure listed in this specification, or other physiological function. Pressure sensor array 369 is connected to stimulating and recording circuit 315 with pressure sensor array connecting cable 370.

In one embodiment of the invention, physiological sensor array 319 comprises cardiovascular sensor array 371 to monitor at least one parameter related to cardiac, cardiovascular, or vascular function or physiology. Example parameters sensed by cardiovascular sensor array 371 comprise intracardiac pressure, right atrium pressure, left atrium pressure, right ventricle pressure, left ventricle pressure, intramural pressure, transmural pressure, pericardial pressure, intraluminal pressure, transvalvular pressure, transthoracic pressure, aortic pressure, pulmonary arterial pressure, central venous pressure, pulmonary venous pressure, arterial pressure, venous pressure, left ventricular end diastolic pressure, LVEDP, intracardiac blood flow, aortic blood flow, pulmonary arterial blood flow, or other pressure or flow related to cardiac function, cardiovascular function, or vascular function. Cardiovascular sensor array 371 may be implemented as at least one of a pressure sensor, flow sensor, Doppler transceiver and/or sensor, acoustic sensor and/or transducer, electrical impedance sensor and/or transducer, mechanical impedance sensor and/or transducer, or other sensor or transducer. Cardiovascular sensor array 371 is connected to stimulating and recording circuit 315 with cardiovascular sensor array connecting cable 372.

In one embodiment of the invention, physiological sensor array 319 comprises glucose sensor array 373 to monitor at least one parameter related to glucose, glycogen, and insulin level and metabolism. Example parameters sensed by glucose sensor array 373 comprise blood glucose level, tissue glucose level, other fluid glucose level, blood glycogen level, tissue glycogen level, other fluid glycogen level, blood insulin level, tissue insulin level, other fluid insulin level, other substance level reflective of levels or metabolism of glucose, glycogen, or insulin. Glucose sensor array 373 may be implemented using chemical, biological, optical, electronic, affinity array, or other known or new technologies for sensing such levels. Glucose sensor array 373 is connected to stimulating and recording circuit 315 with glucose sensor array connecting cable 374.

In one embodiment of the invention, physiological sensor array 319 comprises sensors to monitor head or body position and movement with respect to gravity; these may include accelerometers, gravity sensors, goniometers, hall effect sensors, or other sensors to measure at least one of head, neck, torso, abdomen, and limb position. Accelerometer may be mounted to any structure or structures that enables it to accurately sense a position or movement. Such structures include, for example, the skull base, calvarium, clavicle, mandible, extraocular structures, soft tissues and vertebrae. Accelerometer is connected to stimulating and recording circuit 315 with an accelerometer connecting cable. Accelerometer may be used to sense body position, such as recumbency, and provide information useful to determine circadian rhythm and sleep-wake cycle.

An electromyography (EMG) sensor array 320 is also included in certain embodiments of the invention. EMG sensor array 320 preferably includes a positive proximal EMG electrode, a reference proximal EMG electrode, and a negative proximal EMG electrode. As one skilled in the relevant art would find apparent, EMG sensor array may include any number of type of electrodes. EMG sensor array 320 is non-implanted overlying muscle tissue or is implanted in or adjacent to muscle tissue. EMG electrode array 320 may be located to sense activity of skeletal muscle, smooth muscle, or cardiac muscle and may therefore be used for many sensory modalities comprising motor function, visceral function including gastrointestinal and alimentary function, respiratory function, cardiac function, and other physiological function.

Acoustic transducer array 336 may also be implemented in the present invention. Acoustic transducer array 336 senses muscle vibration and may be used to augment, supplement or replace EMG recording. Also, acoustic transducer array 336 may be used to sense movement, including tremor and voluntary activity. Acoustic transducer array 336 may be used to sense respiratory function, including onset of symptoms of asthma.

It should also be understood from the preceding description that the number of each type of sensor may also be increased or decreased, some sensor types may be eliminated, and other sensor types may be included without departing from the spirit of the present invention.

F. System/Pulse Generator Design.

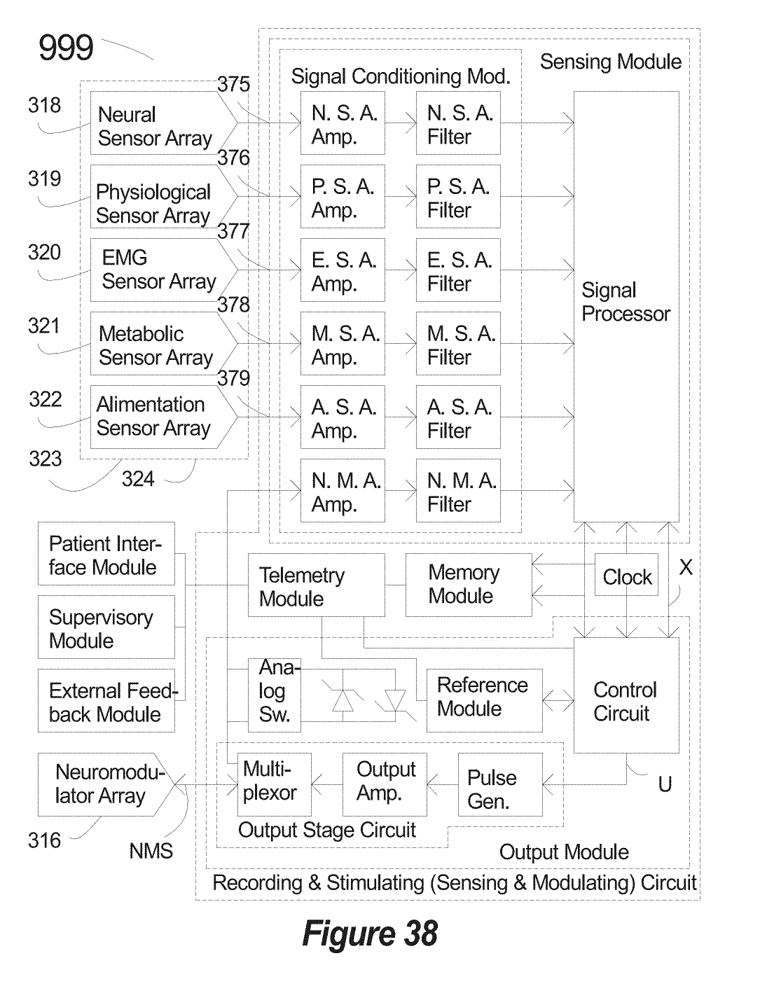

FIG. 38 is an architectural block diagram of one embodiment of the neurological control system 999 of the present invention for modulating the activity of at least one nervous system component in a patient. As used herein, a nervous system component includes any component or structure comprising an entirety or portion of the nervous system, or any structure interfaced thereto. In one preferred embodiment, the nervous system component that is controlled by the present invention includes the sympathetic nervous system. In another preferred embodiment, the controlled nervous system component is the parasympathetic nervous system. In yet another preferred embodiment, the controlled nervous system component is at least one component of the hypothalamus. In an additional preferred embodiment, the controlled nervous system component is at least one component of the pituitary.

Stimulating and recording unit 362, comprises stimulating and recording circuit 315, circuit enclosure 361, header 317, and a single or plurality of attachment fixture 4 and 5. Stimulating and recording unit 362 is also a preferred embodiment of implantable pulse generator 99, 100, 101, 102, which are understood to be implanted or alternatively nonimplanted.

The neurological control system 999 includes one or more implantable or noninvasive components including one or more sensors each configured to sense a particular characteristic indicative of a neurological, psychiatric, or metabolic condition.

G. Stimulation Parameters

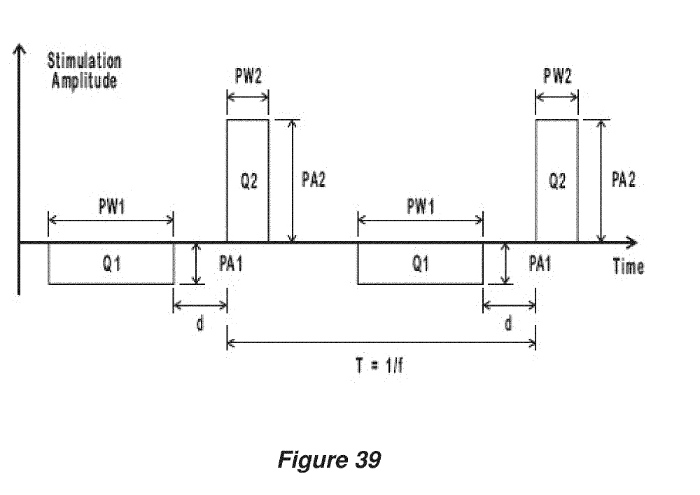

FIG. 39 is a schematic diagram of electrical stimulation waveforms for neural modulation. The illustrated ideal stimulus waveform is a charge balanced biphasic current controlled electrical pulse train. Two cycles of this waveform are depicted, each of which is made of a smaller cathodic phase followed, after a short delay, by a larger anodic phase. In one preferred embodiment, a current controlled stimulus is delivered; and the “Stimulus Amplitude” represents stimulation current. A voltage controlled or other stimulus may be used without departing from the present invention. Similarly, other waveforms, including an anodic phase preceding a cathodic phase, a monophasic pulse, a triphasic pulse, multiphasic pulse, or the waveform may be used without departing from the present invention.

The amplitude of the first phase, depicted here as cathodic, is given by pulse amplitude 1 PA1; the amplitude of the second phase, depicted here as anodic, is given by pulse amplitude 2 PA2. The durations of the first and second phases are pulse width 1 PW1 and pulse width 1 PW2, respectively. Phase 1 and phase 2 are separated by a brief delay d. Waveforms repeat with a stimulation period T, defining the stimulation frequency as f=1/T.

The area under the curve for each phase represents the charge Q transferred, and in the preferred embodiment, these quantities are equal and opposite for the cathodic (Q1) and anodic (Q2) pulses, i.e. Q=Q1=Q2. For rectangular pulses, the charge transferred per pulse is given by Q1=PA1*PW1 and Q2=PA2*PW2. The charge balancing constraint given by −Q1=Q2 imposes the relation PA1*PW1=−PA2*PW2. Departure from the charge balancing constraint, as is desired for optimal function of certain electrode materials, in included in the present invention.

The stimulus amplitudes PA1 and PA2, durations PW1 and PW2, frequency f, or a combination thereof may be varied to modulate the intensity of the said stimulus. A series of stimulus waveforms may be delivered as a burst, in which case the number of stimuli per burst, the frequency of waveforms within the said burst, the frequency at which the bursts are repeated, or a combination thereof may additionally be varied to modulate the stimulus intensity.

Typical values for stimulation parameters include f=100-300 Hz, PA1 and PA2 range from 10 microamps to 10 milliamps, PW1 and PW2 range from 50 microseconds to 100 milliseconds. These values are representative, and departure from these ranges is included in the apparatus and method of the present invention.

Safe stimulation current waveforms may be achieved for stimulus waveforms which satisfy charge injection limits. For stimulation of peripheral nerves, sympathetic nerves, sympathetic trunk, sympathetic plexus, vagus nerve, and other neural structures, such as may be performed using peripheral nerve interface 380, charge injection limits may be selected to be approximately or less than 50 microcoulombs per square centimeter for stainless steel electrodes and approximately or less than 25 microcoulombs per square centimeter for Platinum-Iridium (Pt/Ir) electrodes.

For a design as shown in FIGS. 8 and 9, in which exposed electrode wire tips comprise the active electrode site, an example set of dimensions for a stainless steel implementation of this electrode are a diameter of 50 microns and an exposed length of 2,000 microns (2 mm), resulting in a gross surface area of 314,000 square microns. This may increase substantially if the surface is roughened. The 50 microcoulomb per square centimeter charge injection limit for such a stainless steel electrode would be 0.157 microcoulombs, which would be satisfied by stimulation waveform of amplitude 1.57 milliamperes and pulse width 100 microseconds. For an example electrode resistance of 4,000 ohms, the required stimulation voltage would be 6.28 volts.

An example set of dimensions for a Platinum-Iridium implementation of this electrode are a diameter of 127 microns and an exposed length of 2,000 microns (2 mm), resulting in a gross surface area of 797,560 square microns. This may increase substantially if the surface is roughened. The 25 microcoulomb per square centimeter charge injection limit for such a Platinum-Iridium electrode would be 0.199 microcoulombs, which would be satisfied by stimulation waveform of amplitude 1.99 milliamperes and pulse width 100 microseconds. For an example electrode resistance of 4,000 ohms, the required stimulation voltage would be 7.97 volts.

These dimensions are for example only, and much larger or smaller electrode dimensions and configurations, including those shown in FIGS. 7, 8, 9, and 10, and other figures in the present invention, and other electrode designs without departing from the present invention.

H. Recording Signals

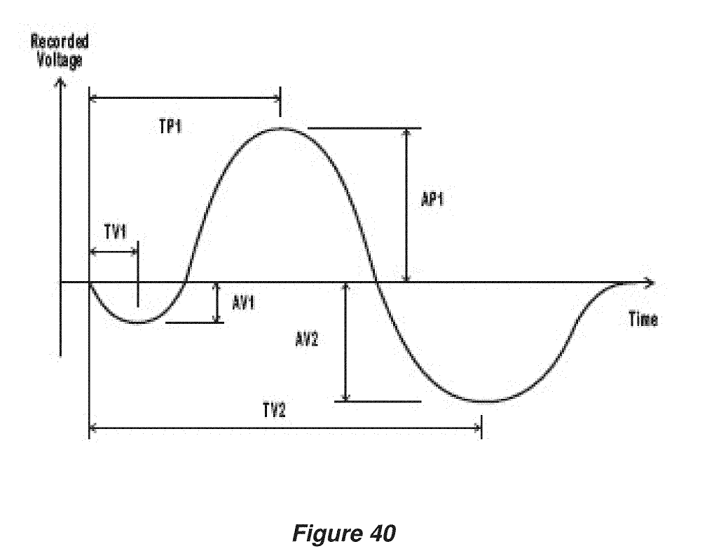

FIG. 40 is a schematic diagram of electrical recording waveforms from neural or muscular structures. These are sensed by any of sensor array 323 and transmitted to recording and stimulation circuit 315 for processing and disease state estimation.

I. Control

In one preferred embodiment, sympathetic index is modulated to control at least one of metabolic rate, body temperature, food intake, blood pressure, heart rate, respiratory gas flow, pulmonary function parameters, cardiac parameters, cardiovascular parameters, vascular parameters, and other parameters.

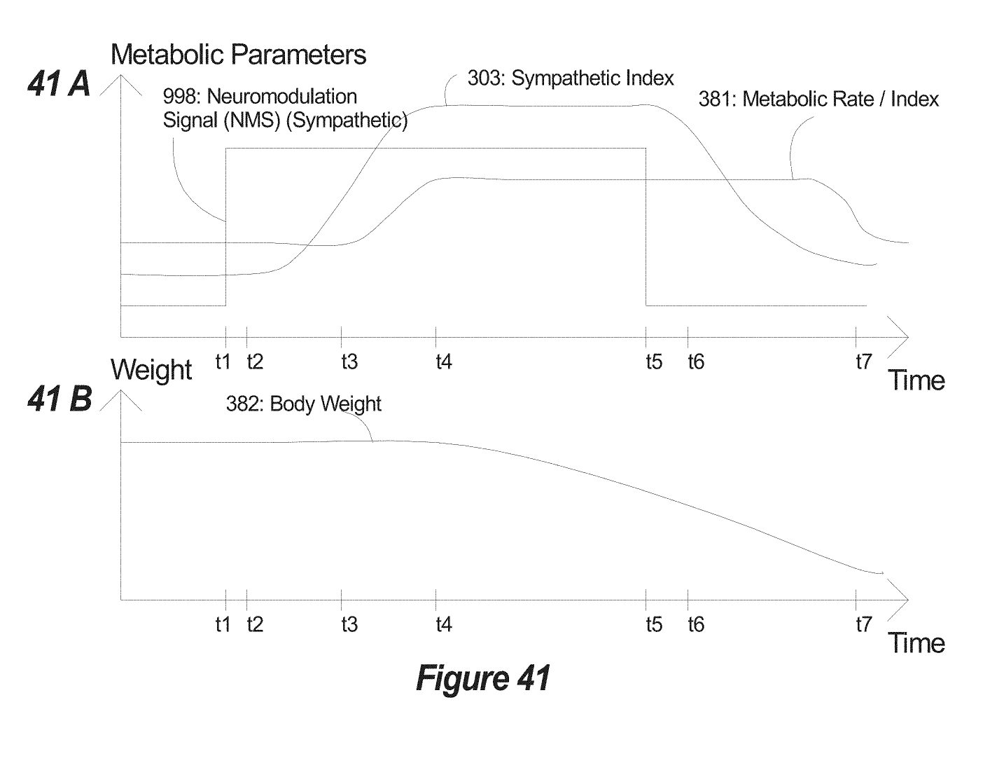

FIG. 41 is a diagram depicting metabolic modulation. Neuromodulation Signal (NMS) is delivered to the sympathetic nervous system, in the sympathetic trunk, splanchnic nerves, celiac plexus, other nerves or plexi, and/or intracranial locations including hypothalamus. NMS causes an increase in sympathetic index, which results in an increase in metabolic rate or metabolic index, which results in a decline in body weight, achieving therapeutic effect in the treatment of obesity.

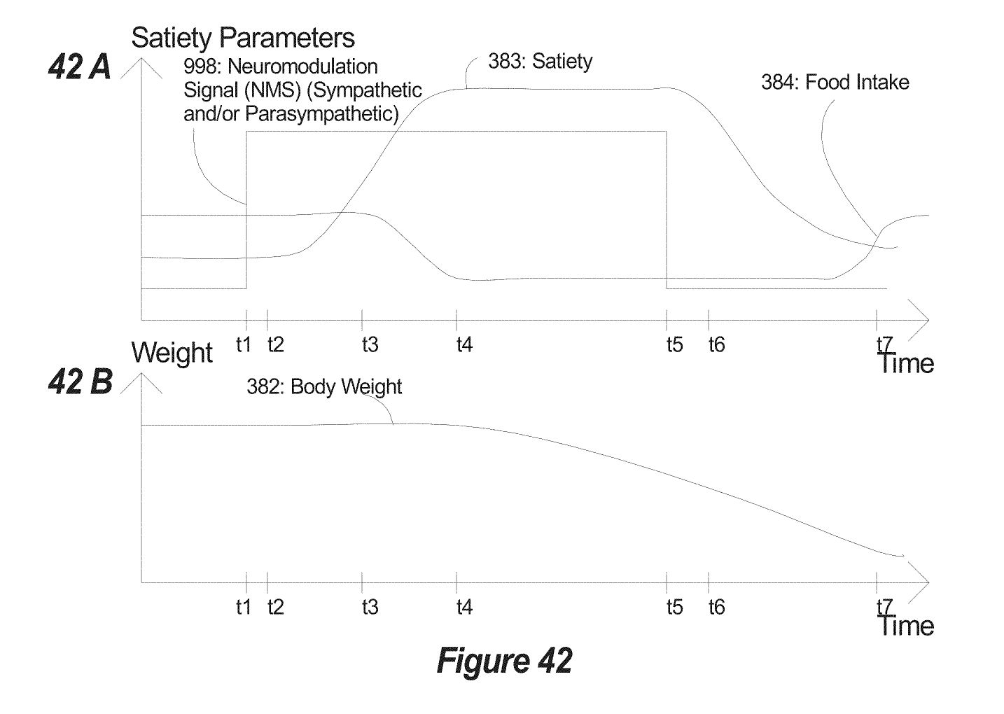

FIG. 42 is a diagram depicting satiety modulation or appetite modulation. Neuromodulation Signal (NMS) is delivered to the autonomic nervous system. The autonomic nervous system includes components of the sympathetic nervous system, including the sympathetic trunk, splanchnic nerves, celiac plexus, other nerves or plexi, and/or intracranial locations including hypothalamus. The autonomic nervous system includes components of the parasympathetic nervous system, including the vagus nerve and parasympathetic afferents, and portions of the solitary nucleus. NMS may cause an increase in parasympathetic index, and causes an increase in satiety, which results in a decrease in food intake, which results in a decline in body weight, achieving therapeutic effect in the treatment of obesity.

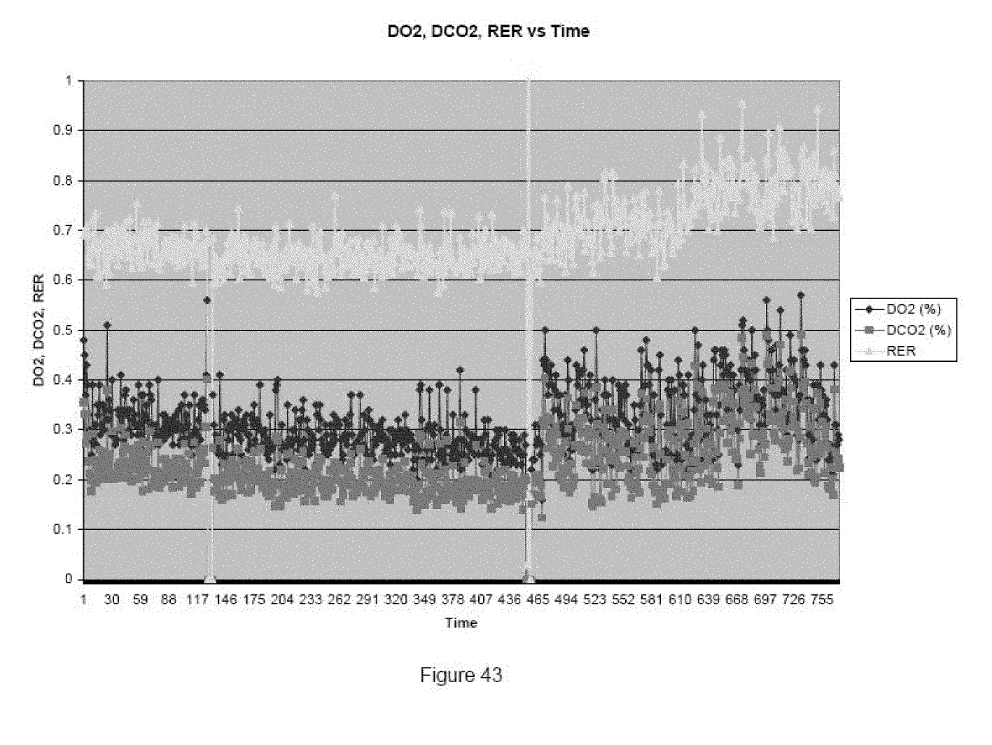

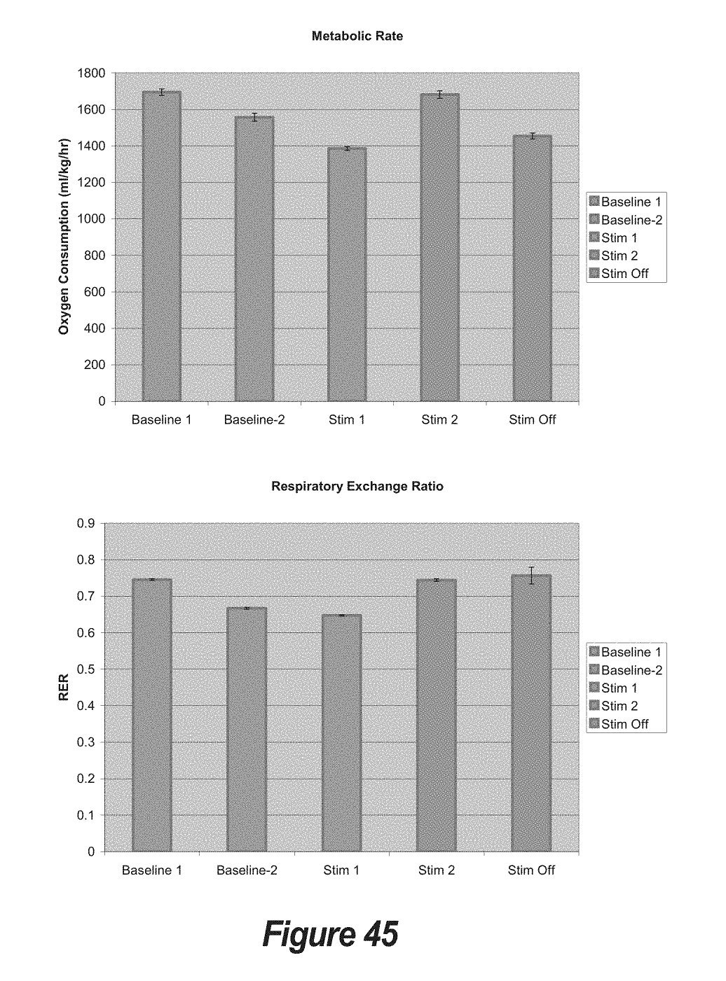

In FIGS. 43, 44, and 45, preclinical animal data is shown for a metabolic experiment in which a rat celiac plexus was stimulated according to the invention taught in the parent case. In this experiment, a cuff electrode constructed from plastic tubing and stainless steel wire was placed around and in communication with the celiac trunk under general anesthesia. Impedance calculations were made to verify integrity of the electrodes prior to implantation to characterize and verify functionality. Stimulation parameters and ranges were chosen to minimize or eliminate stimulation-induced neural tissue damage. Stimuli for this experiment included amplitude of 6 volts, frequency of 60 cycles per second, and a pulse width of 100 microseconds. The second two days of stimulation and the two days of post stimulation had a physiologic respiratory quotient and were used to compare metabolic rate on and off stimulation. The two days on stimulation gave a metabolic rate of 1681+359 ml/kg/hr. The two days post stimulation gave a metabolic rate of 1454+284 ml/kg/hr. The period of stimulation had a 15.6% higher metabolic rate compared to the post-stimulation period (p<0.001). The upper and lower plots in FIG. 45 show the metabolic rates and the respiratory quotients over the days of the experiment.

In FIG. 43, data for DO2, DCO2, RER is plotted versus time.

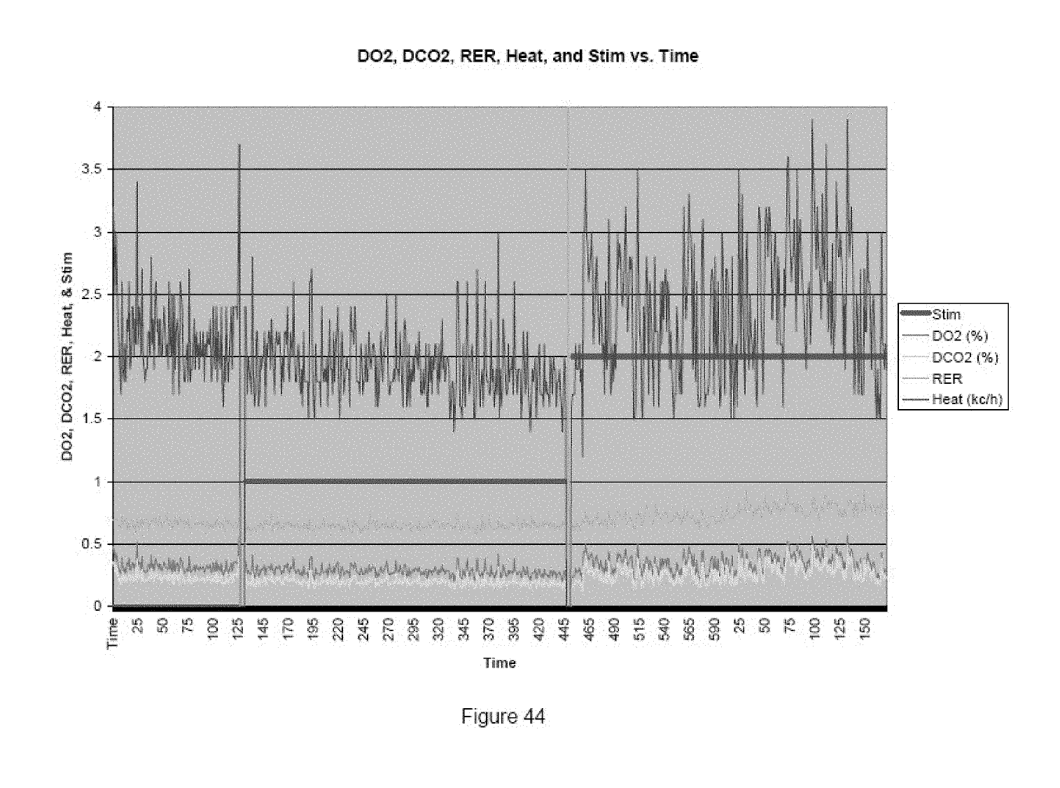

In FIG. 44, data for DO2, DCO2, RER, Heat, and Stimulus Intensity is plotted versus Time.

In FIG. 45, bar graphs for Metabolic Rate and Respiratory Exchange Ratio (RER) are shown versus time for each of the respective bins: Baseline 1, Baseline 2, Stim 1, Stim 2, Stim Off.

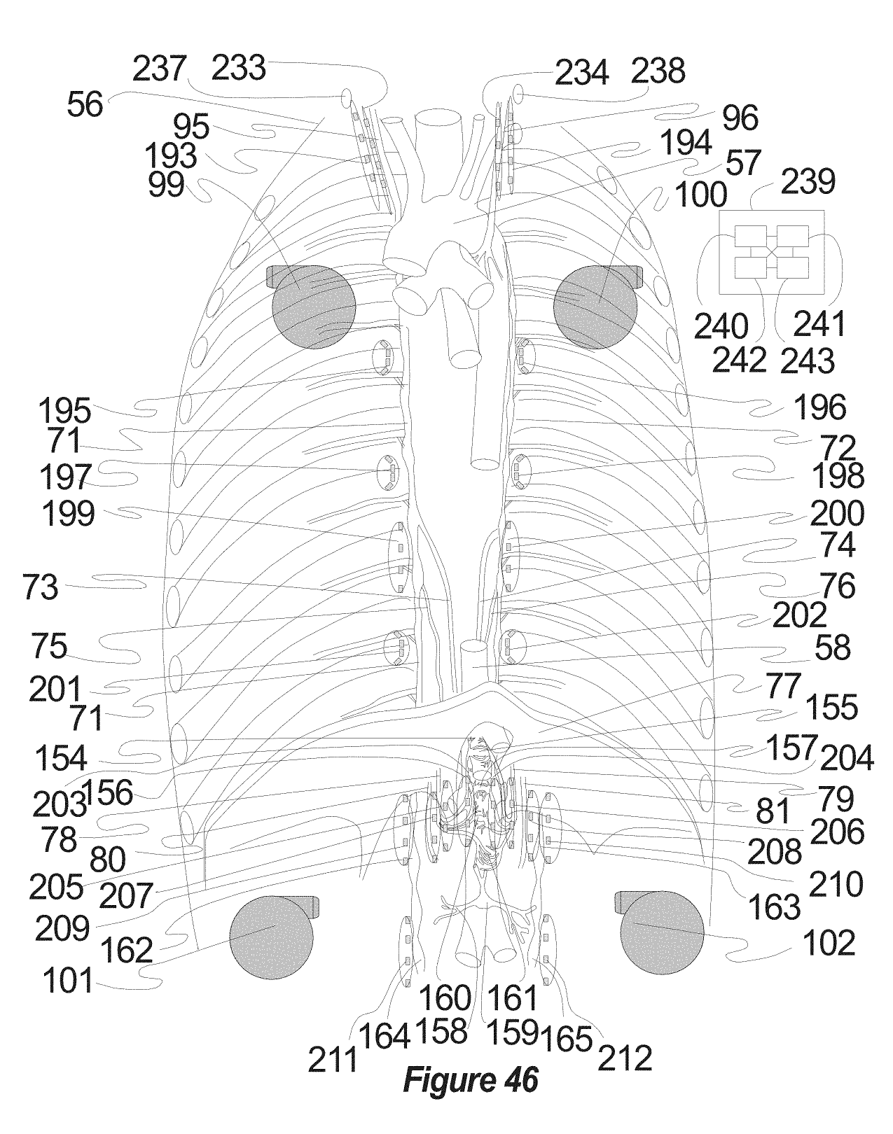

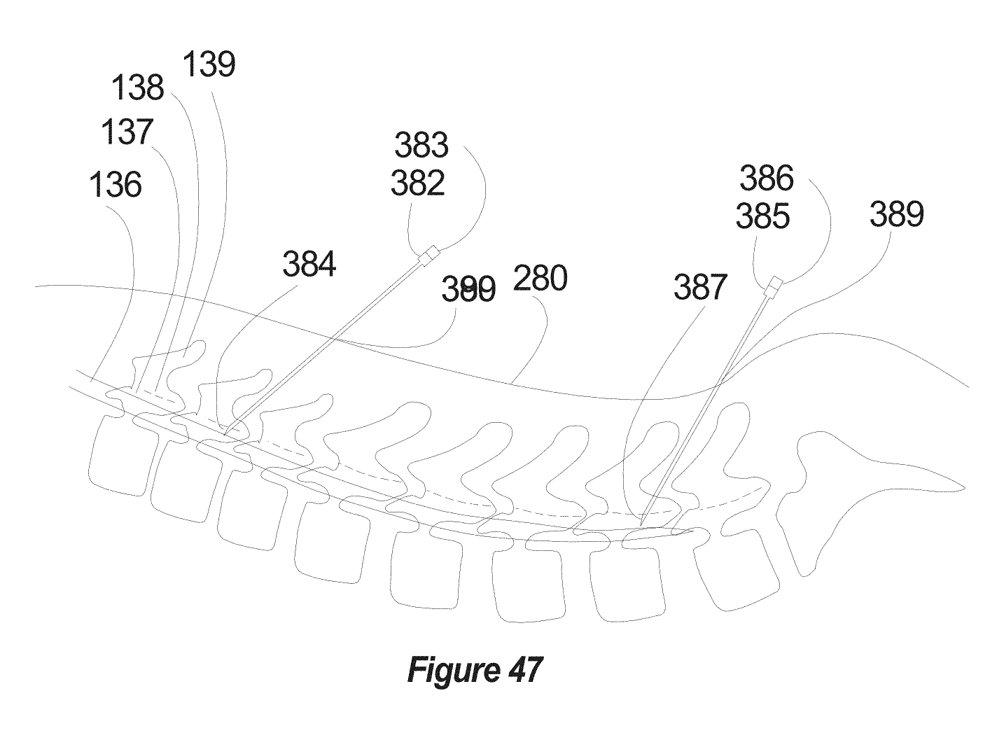

FIG. 46 shows the same invention taught in the parent case and shown in FIG. 16 and in FIG. 30, with detail shown for a telemetrically powered linear catheter-based electrode implementation for the neuromodulatory interfaces. In this FIG. 46, the distal portion of the sympathetic nervous system is shown in more detail. In the parent case, modulation of the sympathetic nervous system was taught for the treatment of disease. This FIG. 46 shows the same neuromodulator configuration shown in FIG. 29, which is a potential arrangement of electrodes that becomes apparent to one skilled in the art upon reading the parent patent specification and figures. Each of the neuromodulator arrays includes a means for bidirectional transmission of information and power to and from at least one of an implantable pulse generator 99. 100, 101, and 102, and an External Transmitting and Receiving Unit 239. Said implantable pulse generator 99. 100, 101, and 102 may also be external to the body and not implanted or may be adjacent to or distant from the skin. Each of the neuromodulator arrays includes a telemetry module, which serves as a means for bidirectional transmission of information and power to and from at least one of an implantable pulse generator 99. 100, 101, and 102 and External Transmitting and Receiving Unit 239. Each of the neuromodulator arrays includes a means for bidirectional transmission of information and power to and from at least one of an External Transmitting and Receiving Unit 239. Each of the implantable pulse generator 99. 100, 101, and 102 includes a means for bidirectional transmission of information and power to and from at least one of an External Transmitting and Receiving Unit 239.

Neuromodulator Arrays comprise designs taught in FIG. 8, FIG. 9, and FIG. 10, and other Neuromodulator Array designs known to those knowledgeable in the art as well as other Neuromodulator Array designs including designs to be developed in the future:

1. Epineural Neuromodulator Arrays/Epineural Neuromodulatory Interfaces: Neuromodulator Arrays include neuromodulators which perform epineural modulation such as that shown in FIG. 7 or other epineural stimulators such as the BION, manufactured by Advanced Bionics and described in Schulman J, et. al, “An implantable Bionic Network of Injectable neural prosthetic devices: The future platform for functional electrical stimulation and sensing to restore movement and sensation”, in Bronzino, J (ed), “The Biomedical Engineering Handbook”, 2006, Taylor and Francis Group, Boca Raton, Fla., pages 34-1 to 34-17, which is hereby incorporated by reference. Epineural Neuromodulator Arrays as described herein further comprise helical epineural electrode designs, such as that manufactured by Cyberonics (Houston, Tex.) and described in U.S. Pat. No. 5,251,634, which is hereby incorporated by reference.

2. Intraneural Neuromodulator Arrays/Intraneural Neuromodulatory Interfaces: Still further Neuromodulator Array designs comprising the invention taught in the present application and the parent applications include Intraneural neural interfaces, such as that taught in FIG. 8 and FIG. 9, and those taught in DiLorenzo, Daniel J., “Development of a Chronically Implanted Microelectrode Array for Intraneural Electrical Stimulation for Prosthetic Sensory Feedback”, S. M. Thesis, Harvard-MIT Division of Health Sciences and Technology (1999), which is hereby incorporated by reference. Additional Neuromodulator Array designs comprising the invention taught in the present application and the parent application include microelectronic intraneural neural interfaces, such as those described in U.S. Pat. No. 5,314,458, “Single channel microstimulator”, which is hereby incorporated by reference. Further Intraneural designs comprising the present invention include fully intraneural electrodes, such as miniature wireless devices which use radiofrequency (RF) or other wireless link for at least one of signal and power and may be fabricated using at least one of Micro-Electro-Mechanical Systems (MEMS) technology, Radiofrequency Identification (RFID) technology, or other technology to produce wirelessly powered Intraneural neuromodulatory interfaces with dimensions of similar to or less than the diameter of a nerve. One preferred geometry is cylindrical, facilitating injection or insertion directly into a nerve, neural structure, or other tissue. Another preferred geometry is another elongated shape with any of various cross sections including oval, square, flat, or other geometry, also facilitating injection or insertion directly into a nerve, neural structure, or other tissue. Yet another preferred geometry is a flat shape with any of various cross-sectional geometries including oval, square, rectangular, triangular, tapered, or other geometry, also facilitating injection or insertion directly into a nerve, neural structure, or other tissue. Such dimensions may be 1 cm or less, such as for a sciatic nerve, down to 5 mm or less for a median nerve or other nerve, down to a fraction of 1 millimeter for smaller nerves. Such intraneural neuromodulatory interfaces may have dimensions down to several microns to enable placement within or between fascicles of a nerve or to enable minimally traumatic placement in the cortex, cerebrum, cerebellum, spinal cord, brachial plexus, sacral plexus, deep brain nucleus, brainstem structure, or other neural region or tissue or structure in communication with the nervous system.

In FIG. 46, Wireless Neuromodulator Arrays comprise at least one of Epineural Neuromodulator Arrays, Epineural Neuromodulatory Interfaces, Intraneural Neuromodulator Arrays, and Intraneural Neuromodulatory Interfaces, or any combination thereof. Specific Neuromodulator Arrays shown include Right Cervical Plexus Neuromodulator Array 193, Left Cervical Plexus Neuromodulator Array 194, Right Intercostal Neuromodulator Array 195, Left Intercostal Neuromodulator Array 196, Right Intercostal Neuromodulator Array 197, Left Intercostal Neuromodulator Array 198, Right Intercostal Neuromodulator Array 199, Left Intercostal Neuromodulator Array 200, Right Intercostal Neuromodulator Array 201, Left Intercostal Neuromodulator Array 202, Right Abdominal Para Plexus Neuromodulator Array 203, Left Abdominal Para Plexus Neuromodulator Array 204, Right Abdominal Superior Splanchnic Neuromodulator Array 205, Left Abdominal Superior Splanchnic Neuromodulator Array 206, Right Abdominal Inferior Splanchnic Neuromodulator Array 207, Left Abdominal Inferior Splanchnic Neuromodulator Array 208, Right Abdominal Sympathetic Trunk Neuromodulator Array 209, Left Abdominal Sympathetic Trunk Neuromodulator Array 210, Right Abdominal Sympathetic Trunk Neuromodulator Array 211, and Left Abdominal Sympathetic Trunk Neuromodulator Array 212. Additional Neuromodulator Arrays may be added without departing from the present invention. Wireless Neuromodulator Arrays may contain a single or plurality of wireless neuromodulators or wireless neuromodulatory interfaces, and may be of similar or different designs.

### Minimally Invasive Surgical Technique

In prior applications in this portfolio, apparatus, methods, and surgical techniques were taught for minimally invasive surgical placement of portions or the entirety of a neuromodulation system for the modulation of sympathetic nervous system and for the modulation of other neural tissues including parasympathetic nervous system, other peripheral nervous structures, spinal cord, and other tissues including muscle tissue and gastrointestinal tissues and layers. Further detail of these and other apparatus, methods, and techniques are described in the present application. Some of this detail was presumed to be known to clinical practitioners, including neurosurgeons, and others skilled in the art, and so was not presented in as much detail in the parent application. The apparatus and method taught in the parent case is enabling to an engineer or engineering team skilled it the art of developing neuromodulation systems, and the anatomical placement of electrodes is enabling to a competent clinical practitioner, who has an armamentarium of surgical implantation techniques at his or her disposal. For sake of completeness, example apparatus and procedures are presented in the present application.

In the parent case, FIG. 17 depicted and labeled anatomical structures and FIG. 18 taught locations and characteristics of neuromodulators shown in communication with various portions of the nervous system. Various apparatus, methods, and surgical techniques may be used to achieve the placement of electrodes in these and other positions taught in the parent and present applications. Other apparatus, methods, and surgical techniques or procedures may be used to place neuromodulators in these or other locations without departing from the present invention. These include percutaneous insertion of electrodes or neuromodulators into various positions such that they are configured to be in communication with portions of the nervous system.

In FIGS. 32 through 37, described previously, a multiplicity of neuromodulators are shown in communications with multiple portions of the nervous system, including the sympathetic nervous system. These portions of the nervous system include but are not limited to the spinal cord 151, the dorsal spinal cord, including the posterior columns, the dorsal spinal root 125, the ventral spinal root 125, the dorsolateral spinal cord, the lateral spinal cord, the intermediolateral columns or intermediolateral nucleus 121, the anterolateral spinal cord, the anterior spinal cord, the spinal nerve 127.

In FIG. 47-49, apparatus and method are shown for the implantation of neuromodulators configured to be in communication with single or multiple components of the nervous system, including but not limited to the spinal cord 151, intermediolateral nucleus 121, spinal cord white matter 122, structure in the region of the anterior median fissure 123, ventral spinal root 124, dorsal spinal root 125, spinal ganglion 126, spinal nerve 127, spinal nerve anterior ramus 128, spinal nerve posterior ramus 129, grey ramus communicantes 130, white ramus communicantes 131, sympathetic trunk 132, other structures accessible via the epidural space 137, ventral horn of spinal gray matter 141, dorsal horn of spinal gray matter 142, and other structures within the spinal cord, adjacent to the spinal cord, in the region of the vertebral column, and elsewhere in or near the body.

FIG. 47 depicts a step in the surgical placement of neuromodulatory interface 391 and 392 configured to be in communication with single or multiple components of the nervous system.

Cannula 382 with stylet 383 inserted within are shown piercing skin 280 in the lumbar region, the intervening structures and the ligamentum flavum 388, with the cannula tip 384 entering the epidural space 137 in the lumbosacral region. Cannula 382 may enter or pierce skin 280 in the lumbar region as shown or anywhere else on the body. In the technique shown in FIG. 47, in which the entry point 389 in the skin 280 is in the region of the trunk overlying the lumbosacral spine, the entry point 389 may be posterior midline, paramidline, or lateral to the spinal column or vertebral bodies, or in the flank, lateral abdomen, anterior abdomen or pelvis, or at any other location and angle of entry on the body.

Cannula 385 with stylet 386 inserted within are shown piercing skin 280 in the thoracic or cervical region, the intervening structures and the ligamentum flavum 388, with the cannula tip 385 entering the epidural space 137 in the cervicothoracic region. Cannula 385 may enter or pierce skin 280 in the thoracic region as shown or cervical region or anywhere else on the body. In the technique shown in FIG. 47, in which the entry point 390 in the skin 280 is in the region of the trunk overlying the thoracic spine as shown or in the cervical region, the entry point 390 may be posterior midline, paramidline, or lateral to the spinal column or vertebral bodies, overlying apportion of the ribs, in the paramanubrial regions, in the cervical region, in the thoracic region, in the axillary region, or other regions which could provide access to neural structures including the intracranial compartment.

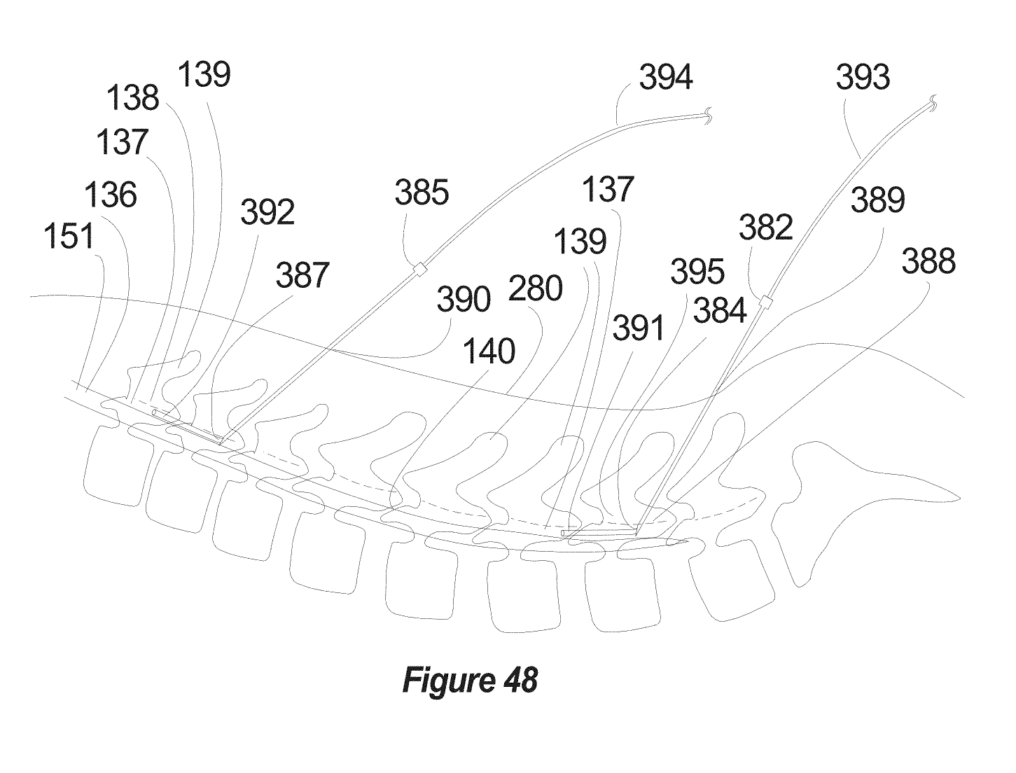

FIG. 48 depicts a step in the surgical placement of neuromodulatory interface 391 and 392 configured to be in communication with single or multiple components of the nervous system.

A dorsal approach procedure is performed as follows. The patient is typically placed prone or in a lateral position on the operating table. The skin comprising and adjacent to the operative site is sterilely prepped and draped. This comprises the dorsal portion of the thoracolumbosacral trunk and extends laterally to the flank and anterolateral abdomen to include the skin overlying the location where a subcutaneous pocket may be made for implantation of the implantable pulse generator (IPG). Using intraoperative x-ray, fluoroscopic, or frameless stereotactic guidance, one or more cannula 382, 385 with stylet 383, 386 inserted are advanced percutaneously through the skin 280. Cannula 382, 385 with stylet 383, 386 are typically inserted through skin 280 with the cannula tip 384, 387 angled in a rostral or cephalic direction (toward the head) and the bevel of the tip angled also in a cephalic direction. For placement in an epidural location, cannula 382, 385 with stylet 383, 386 are typically advanced either midline between the vertebral spinous process 139 (shown in cross section in FIG. 17) of adjacent vertebrae or paramidline lateral to vertebral spinous process 139 and medial to vertebral facet 140 and in between adjacent vertebral lamina 395. Cannula 382, 385 with stylet 383, 386 are advanced through the ligamentum flavum and into the spinal canal. Care is usually taken to advance the tip of the cannula 382, 385 through the ligamentum flavum 388 into the epidural space 137 and to avoid penetrating the meningeal layer of dura mater 136 (anatomy also shown in cross section in FIGS. 17 and 18). Stylet 383, 386 is then withdrawn from cannula 382, 385. Alternatively, during the process of inserting cannula 382, 385, a syringe with normal saline solution is attached to cannula 382, 385; and gentle pressure is applied to the syringe piston, such that once the cannula 382, 385 tip enters the epidural space 137, normal saline begins to flow from the syringe through cannula 382, 385 and into the epidural space 137. The consequent drop in pressure in the syringe can be sensed by the surgeon as a reduction in back force form the syringe on the surgeon's thumb, confirming entry into the epidural space 137.

After the stylet 383, 386 or syringe is removed from the cannula 382, 385, neuromodulatory interface 391, 392 is advanced through cannula 382, 385 into the desired position. Neuromodulatory interface 391, 392 may be at the tip or elsewhere along a connecting cable 393, 394 or Neuromodulatory interface 391, 392 may be separate and wireless. Neuromodulatory interface 391, 392 with connecting cable 393, 394 may be advanced through cannula 382, 385, and the desired position is usually in the epidural space 137, though it could be positioned outside the spinal canal or within the meningeal layer of dura mater 136 or within the spinal cord 151. By manipulating the position and angle of entry of the cannula 382, 385 in the transverse plane and by moving the cannula 382, 385 and twisting the connecting cable 393, 394, the surgeon can direct the positioning of the neuromodulatory interface 391, 392.

Using this technique described or other technique for the placement neuromodulatory interface 391, 392, one may implant one or more of anterior central spinal neuromodulatory interface 143, anterior right lateral spinal neuromodulatory interface 144, anterior left lateral spinal neuromodulatory interface 145, right lateral spinal neuromodulatory interface 149, left lateral spinal neuromodulatory interface 150, posterior central spinal neuromodulatory interface 146, posterior right lateral spinal neuromodulatory interface 147, posterior left lateral spinal neuromodulatory interface 148, and neuromodulatory interface 391, 392 of other design and configured to be implanted in other positions. By advancing cannula 382, 385 through meningeal layer of dura mater 136, the intermediolateral nucleus neuromodulatory interface 152 may be implanted within the spinal cord 151 in the intermediolateral nucleus 121 (shown in cross section in FIG. 17 and FIG. 18) or other component of the nervous system

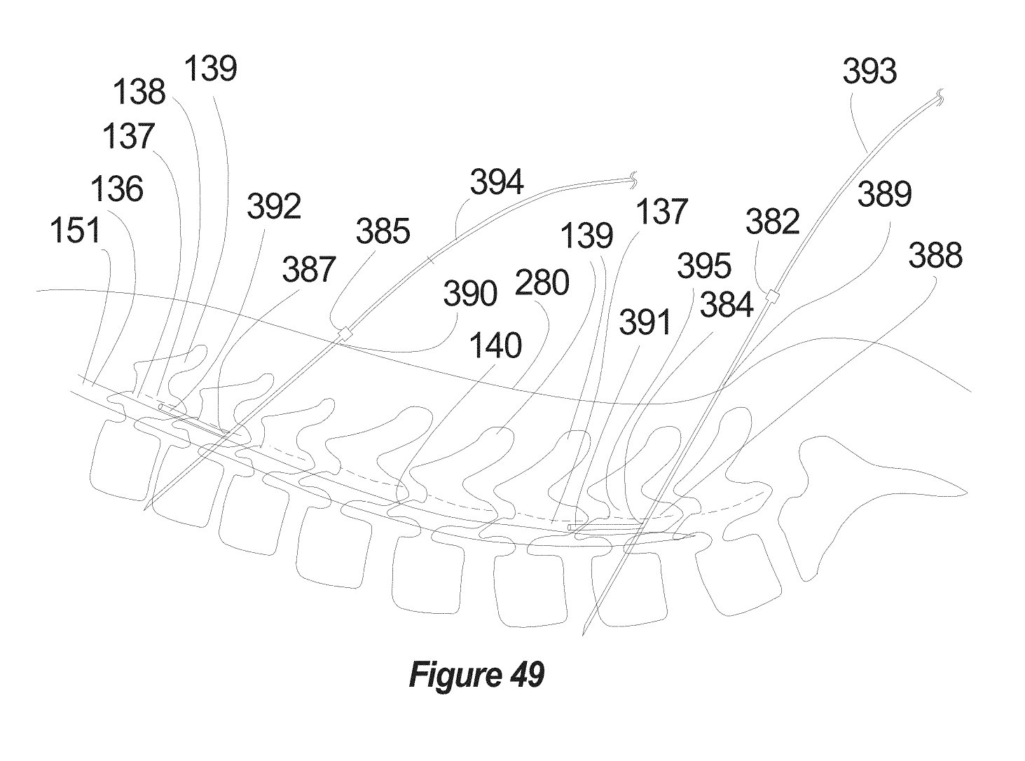

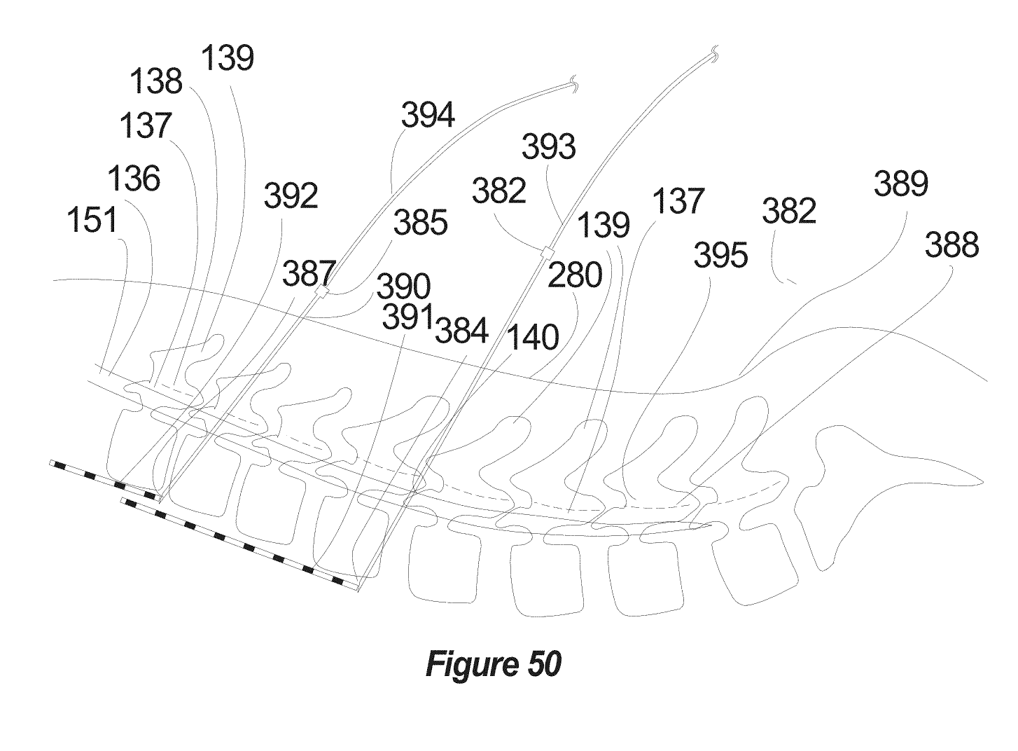

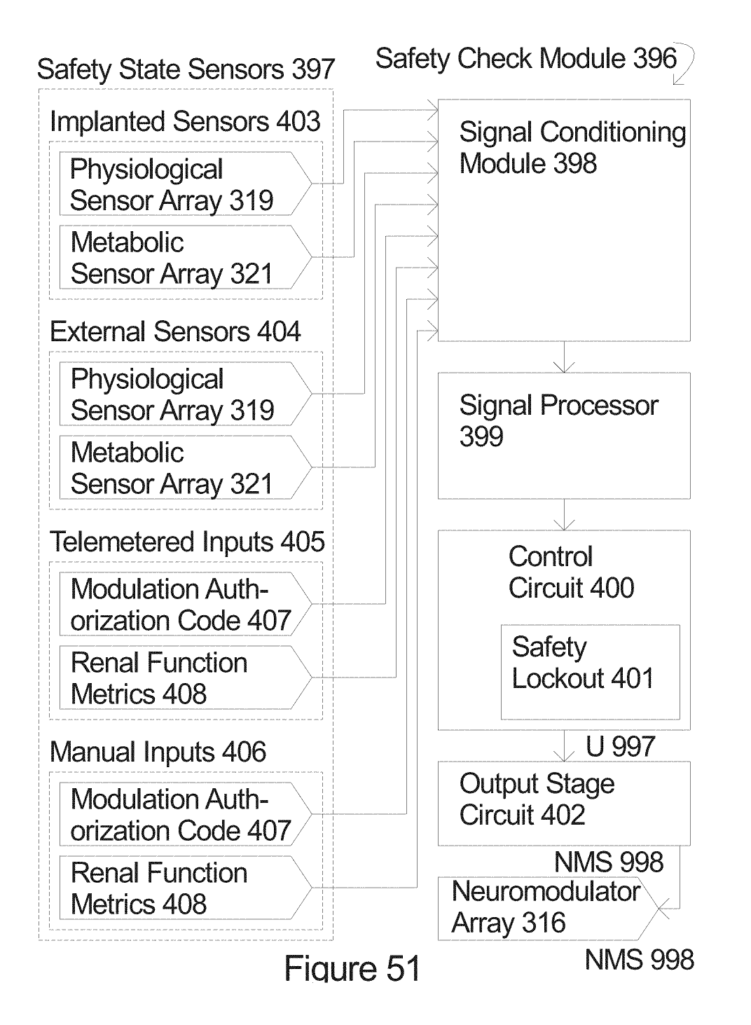

FIG. 49 and FIG. 50 depict steps in the surgical placement of neuromodulatory interface 391 and 392 configured to be in communication with single or multiple components of the nervous system.

A dorsal approach procedure is performed as follows. The patient is typically placed prone or in a lateral position on the operating table. The skin comprising and adjacent to the operative site is sterilely prepped and draped. This comprises the dorsal portion of the thoracolumbosacral trunk and extends laterally to the flank and anterolateral abdomen to include the skin overlying the location where a subcutaneous pocket may be made for implantation of the implantable pulse generator (IPG). Using intraoperative x-ray, fluoroscopic, or frameless stereotactic guidance, one or more cannula 382, 385 with stylet 383, 386 inserted are advanced percutaneously through the skin 280. Cannula 382, 385 with stylet 383, 386 may be inserted through skin 280 with the cannula tip 384, 387 angled perpendicular to the skin, in a rostral or cephalic direction (toward the head), caudally toward the feet, laterally, or medially from a paramidline entry point.

This technique may be used for placement of neuromodulatory interface 391 and 392 in communication with a neural plexus or component of the nervous system, including but not limited to the celiac plexus 154, Superior Mesenteric Plexus 156, Renal Plexus 159, Inferior Mesenteric Plexus 160, Iliac Plexus 161, Right Lumbar Sympathetic Ganglia 162, Left Lumbar Sympathetic Ganglia 163, Right Sacral Sympathetic Ganglia 164, Left Sacral Sympathetic Ganglia 165, and other neural structures and body tissues.

Using the techniques implied in the present figures and depicted in more detail in FIGS. 33 and 34 and in FIGS. 47 through 50 or using other techniques, one may implant or place a single or multiplicity of neuromodulatory interfaces, including but not limited to one of the following: modulator 2, modulator 3, Modulator 24, Modulator 25, Modulator, 26, Modulator 27, Modulator 28, Modulator 29, Modulator 30, Modulator 31, Neuromodulatory interface 34, Longitudinal Electrode Array 38, Regeneration Tube Array 42, Regeneration Tube 43, Regeneration Electrode Array 44, Vagus Nerve Interface 45, Sympathetic Nerve Interface 46, Epineural Electrode 49, Epineural Electrode 50, Epineural Electrode 51, Sympathetic Trunk Neuromodulatory Interface 83, Sympathetic Trunk Neuromodulatory Interface 84, Sympathetic Trunk Neuromodulatory Interface 85, Sympathetic Trunk Neuromodulatory Interface 86, Thoracic Splanchnic Neuromodulatory Interface 87, Thoracic Splanchnic Neuromodulatory Interface 88, Thoracic Splanchnic Neuromodulatory Interface 89, Thoracic Splanchnic Neuromodulatory Interface 90, Abdominal Splanchnic Neuromodulatory Interface [Right Greater] 91, Abdominal Splanchnic Neuromodulatory Interface [Left Greater] 92, Abdominal Splanchnic Neuromodulatory Interface [Right Lesser] 93, Abdominal Splanchnic Neuromodulatory Interface [Left Lesser] 94, Vagus Neuromodulatory Interface 97, Vagus Neuromodulatory Interface 98, Epineural Cuff Electrode Neuromodulatory Interface 117, Longitudinal Electrode Neuromodulatory Interface 118, Longitudinal Electrode Regeneration Port Neuromodulatory Interface 119, Regeneration Tube Neuromodulatory Interface 120, Anterior Central Spinal Neuromodulatory Interface 143, Anterior Right Lateral Spinal Neuromodulatory Interface 144, Anterior Left Lateral Spinal Neuromodulatory Interface 145, Posterior Central Spinal Neuromodulatory Interface 146, Posterior Right Lateral Spinal Neuromodulatory Interface 147, Posterior Left Lateral Spinal Neuromodulatory Interface 148, Right Lateral Spinal Neuromodulatory Interface 149, Left Lateral Spinal Neuromodulatory Interface 150, Intermediolateral Nucleus Neuromodulatory Interface 152, Abdominal Splanchnic Neuromodulatory Interface 166, Abdominal Splanchnic Neuromodulatory Interface 167, Abdominal Splanchnic Neuromodulatory Interface 170, Abdominal Splanchnic Neuromodulatory Interface 171, Neuromodulator Array 174, Neuromodulator Array 175, Abdominal Splanchnic Neuromodulatory Interface 178, Abdominal Splanchnic Neuromodulatory Interface 179, Abdominal Splanchnic Neuromodulatory Interface 180, Abdominal Splanchnic Neuromodulatory Interface 181, Abdominal Splanchnic Neuromodulatory Interface 182, Abdominal Splanchnic Neuromodulatory Interface 183, Abdominal Splanchnic Neuromodulatory Interface 184, Abdominal Splanchnic Neuromodulatory Interface 185, Abdominal Splanchnic Neuromodulatory Interface 186, Right Cervical Plexus Neuromodulator Array 193, Left Cervical Plexus Neuromodulator Array 194, Right Intercostal Neuromodulator Array 195, Left Intercostal Neuromodulator Array 196, Right Intercostal Neuromodulator Array 197, Left Intercostal Neuromodulator Array 198, Right Intercostal Neuromodulator Array 199, Left Intercostal Neuromodulator Array 200, Right Intercostal Neuromodulator Array 201, Left Intercostal Neuromodulator Array 202, Right Abdominal Para Plexus Neuromodulator Array 203, Left Abdominal Para Plexus Neuromodulator Array 204, Right Abdominal Superior Splanchnic Neuromodulator Array 205, Left Abdominal Superior Splanchnic Neuromodulator Array 206, Right Abdominal Inferior Splanchnic Neuromodulator Array, 207, Left Abdominal Inferior Splanchnic Neuromodulator Array 208, Right Abdominal Sympathetic Trunk Neuromodulator Array 209, Left Abdominal Sympathetic Trunk Neuromodulator Array 210, Right Abdominal Sympathetic Trunk Neuromodulator Array 211, Left Abdominal Sympathetic Trunk Neuromodulator Array 212, Right Vagal Neuromodulator Array 233, Left Vagal Neuromodulator Array 234, Right Superficial Cardiac Plexus Neuromodulator Array 250, Left Superficial Cardiac Plexus Neuromodulator Array 251, Right Deep Cardiac Plexus Neuromodulator Array 252, Left Deep Cardiac Plexus Neuromodulator Array 253, Right Renal Plexus Neuromodulator Array 254, Left Renal Plexus Neuromodulator Array 255, Right Renal Nerve Branch Neuromodulator Array 256, Left Renal Nerve Branch Neuromodulator Array 257, Right Anterior Pulmonary Nerve Neuromodulator Array 266, Left Anterior Pulmonary Nerve Neuromodulator Array 267, Neuromodulatory Interface 296, Neuromodulatory Interface 297, Neuromodulatory Interface 298, Neuromodulatory Interface 299, Neuromodulatory Interface Array 301, Neuromodulator Array 316, Neural Sensor Array 318, Physiological Sensor Array 319, EMG Sensor Array 320, Metabolic Sensor Array 321, Alimentation Sensor Array 322, Sensor Array 323, Neuromodulator Array 325, Neuromodulator Array 326, Neuromodulator Array 327, Neuromodulator Array 328, Neuromodulator Array 329, Neuromodulator Array 330, Neuromodulator Array 331, Neuromodulator Array 332, Physiological Sensor Array 333, Physiological Sensor Array 334, Physiological Sensor Array 335, Acoustic Transducer Array 336, Temperature Sensor Array 365, Respiratory Sensor Array 367, Pressure Sensor Array 369, Cardiovascular Sensor Array 371, Glucose Sensor Array 373, Peripheral Nerve Interface 380, Intraneural Interface Array 381, Neuromodulatory Interface 391, Neuromodulatory Interface 392, and other neuromodulatory interface or tissue interface for sensing, delivering an output signal, or other purpose.

Using the techniques implied in the present figures and depicted in more detail in FIGS. 33 and 34 and in FIGS. 47 through 50 or using other techniques, one may implant or place a single or multiplicity of neuromodulatory interfaces in communication with at least one of the following: Stomach 8, Gastric Fundus 9, Greater Curvature of Stomach 10, Pyloric Antrum 11, Gastric Pylorus 12, Duodenum 13, Lower Esophageal Sphincter 14, Esophagus 15, Cardiac Notch of Stomach 16, Lesser Curvature of Stomach 17, Nerve 35, Transected Nerve End 37, Vagus Nerve 47, Sympathetic Nerve Branch 48, Intercostal Nerve 69, Intercostal Nerve 70, Right Sympathetic Trunk 71, Left Sympathetic Trunk 72, Right Greater Splanchnic Nerve 73, Left Greater Splanchnic Nerve 74, Right Lesser Splanchnic Nerve 75, Left Lesser Splanchnic Nerve 76, Right Subdiaphragmatic Greater Splanchnic Nerve 78, Left Subdiaphragmatic Greater Splanchnic Nerve 79, Right Subdiaphragmatic Lesser Splanchnic Nerve 80, Left Subdiaphragmatic Lesser Splanchnic Nerve 81, Right Vagus Nerve 95, Left Vagus Nerve 96, Intermediolateral Nucleus 121, Spinal Cord White Matter 122, Anterior Median Fissure 123, Ventral Spinal Root 124, Dorsal Spinal Root 125, Spinal Ganglion 126, Spinal Nerve 127, Spinal Nerve Anterior Ramus 128, Spinal Nerve Posterior Ramus 129, Grey Ramus Communicantes 130, White Ramus Communicantes 131, Sympathetic Trunk 132, Ventral Horn of Spinal Gray Matter 141, Dorsal Horn of Spinal Gray Matter 142, Spinal Cord 151, Celiac Plexus 154, Celiac Ganglion 155, Superior Mesenteric Plexus 156, Superior Mesenteric Ganglion 157, Renal Plexus 158, Renal Ganglion 159, Inferior Mesenteric Plexus 160, Iliac Plexus 161, Right Lumbar Sympathetic Ganglia 162, Left Lumbar Sympathetic Ganglia 163, Right Sacral Sympathetic Ganglia 164, Left Sacral Sympathetic Ganglia 165, Abdominal Neural Plexus 187, Abdominal Neural Ganglion 188, Right Lumbar Sympathetic Trunk 189, Left Lumbar Sympathetic Trunk 190, Right Cervical Plexus 237, Left Cervical Plexus 238, Superficial Cardiac Plexus 244, Deep Cardiac Plexus 245, Right Anterior Pulmonary Nerve 246, Left Anterior Pulmonary Nerve 247, Right Renal Nerve Branch 248, Left Renal Nerve Branch 249, other branches of these structures, and other sympathetic structures, and other parasympathetic structures, and other neural structures, and other neural structures and body tissues.

By this technique, one may implant or place:
250 Right Superficial Cardiac Plexus Neuromodulator Array
251 Left Superficial Cardiac Plexus Neuromodulator Array
252 Right Deep Cardiac Plexus Neuromodulator Array
253 Left Deep Cardiac Plexus Neuromodulator Array
254 Right Renal Plexus Neuromodulator Array
255 Left Renal Plexus Neuromodulator Array
256 Right Renal Nerve Branch Neuromodulator Array
257 Left Renal Nerve Branch Neuromodulator Array

And other arrays into position to be in communication with the above listed structures or other desired targets.

To target an epidural location, cannula 382, 385 with stylet 383, 386 are typically advanced either midline between the vertebral spinous process 139 (shown in cross section in FIG. 17) of adjacent vertebrae or paramidline lateral to vertebral spinous process 139 and medial to vertebral facet 140 and in between adjacent vertebral lamina 395. Cannula 382, 385 with stylet 383, 386 are advanced through the ligamentum flavum and into the spinal canal. Care is usually taken to advance the tip of the cannula 382, 385 through the ligamentum flavum 388 into the epidural space 137 and to avoid penetrating the meningeal layer of dura mater 136 (anatomy also shown in cross section in FIGS. 17 and 18). Stylet 383, 386 is then withdrawn from cannula 382, 385. Alternatively, during the process of inserting cannula 382, 385, a syringe with normal saline solution is attached to cannula 382, 385; and gentle pressure is applied to the syringe piston, such that once the cannula 382, 385 tip enters the epidural space 137, normal saline begins to flow from the syringe through cannula 382, 385 and into the epidural space 137. The consequent drop in pressure in the syringe can be sensed by the surgeon as a reduction in back force form the syringe on the surgeon's thumb, confirming entry into the epidural space 137.

After the stylet 383, 386 or syringe is removed from the cannula 382, 385, neuromodulatory interface 391, 392 is advanced through cannula 382, 385 into the desired position. Neuromodulatory interface 391, 392 may be at the tip or elsewhere along a connecting cable 393, 394 or Neuromodulatory interface 391, 392 may be separate and wireless. Neuromodulatory interface 391, 392 with connecting cable 393, 394 may be advanced through cannula 382, 385, and the desired position is usually in the epidural space 137, though it could be positioned outside the spinal canal or within the meningeal layer of dura mater 136 or within the spinal cord 151. By manipulating the position and angle of entry of the cannula 382, 385 in the transverse plane and by moving the cannula 382, 385 and twisting the connecting cable 393, 394, the surgeon can direct the positioning of the neuromodulatory interface 391, 392.

Using this technique described or other technique for the placement neuromodulatory interface 391, 392, one may implant one or more of anterior central spinal neuromodulatory interface 143, anterior right lateral spinal neuromodulatory interface 144, anterior left lateral spinal neuromodulatory interface 145, right lateral spinal neuromodulatory interface 149, left lateral spinal neuromodulatory interface 150, posterior central spinal neuromodulatory interface 146, posterior right lateral spinal neuromodulatory interface 147, posterior left lateral spinal neuromodulatory interface 148, and neuromodulatory interface 391, 392 of other design and configured to be implanted in other positions. By advancing cannula 382, 385 through meningeal layer of dura mater 136, the intermediolateral nucleus neuromodulatory interface 152 may be implanted within the spinal cord 151 in the intermediolateral nucleus 121 (shown in cross section in FIG. 17 and FIG. 18) or other component of the nervous system.

In FIG. 50, neuromodulatory interface 391, 392 are shown advancing past the catheter tips 384 and 387 of catheters 382 and 385, respectively. In relation to the long axis of catheter 382 and 385, catheter tips 384 and 387 may be flat or orthogonal, angled or beveled, curved or arched, or configured in any of several manners apparent to those skilled in the art to direct the trajectory and location of neuromodulatory interface 391, 392. Using the taught apparatus and methods, surgeon can direct the trajectory and resulting positioning of neuromodulatory interface 391, 392. Neuromodulatory interface 391, 392 can be advanced in a transverse plane to arch over at least one of the ipsilateral, the anterior, and contralateral, and the posterior portion of the vertebral body and associated structures. Alternatively, or in addition, neuromodulatory interface 391, 392 can be advanced with an axial component, including but not limited to rostrally, superiorly, caudally, inferiorly, such that neuromodulatory interface 391, 392 traverses at least one vertebral body level. Using this technique, neuromodulatory interface 391, 392 can be advanced to cross a single or multiplicity of vertebral levels, comprising at least one within the range comprised by the set C1, C2, C3, C4, C5, C6, C7, T1, T2, T3, T4, T5, T6, T7, T8, T9, T10, T11, L2, L2, L3, L4, L5, S1, S2, S3, S4, S5, and Cx. By this technique, facilitated by the apparatus and methods teachings contained herein, neuromodulatory interface 391, 392 may be positioned across a sufficiently wide region to compensate for normal anatomical and physiological variability as well as for abnormal anatomical and physiological variability among patients and users of this system. A single or plurality of neuromodulatory interface 391, 392 may be implanted or positioned in a minimally or noninvasive manner. Neuromodulatory interfaces 391, 392 may be implanted or positioned such that they are ipsilateral or contralateral to each other. Neuromodulatory interfaces 391, 392 may be implanted or positioned such that the spinal levels with which they overlap or are adjacent or interface are mutually fully overlapping, partially overlapping, non-overlapping, or distinct. Neuromodulatory interfaces 391, 392 are depicted in FIG. 50 as comprising linear arrays of neuromodulators. Neuromodulatory interfaces 391, 392 may comprise a single or plurality of neuromodulators without departing from the present invention. Neuromodulatory interfaces 391, 392 may comprise a single or plurality of electrodes, electrical transducers, ultrasonic transducers, sonic transducers, high frequency ultrasonic (HIFU) transducers, vibratory transducers, optical transducers, thermal transducers, chemical transducers, pharmacological transducers, or other transducers, actuators, and/or sensors without departing from the present invention.

Multimodal Targeting: As taught in the present invention, neuromodulatory interfaces may be placed at locations to modulate targets which induce satiety (for example with spinal cord stimulation, including but not limited to levels T5, T6, T7, and other cervical, thoracic, lumbar, sacral levels, midline and other positions) and to modulate targets which increase metabolism (including the splanchnic nerves and their branches and nerve roots as they enter and exit the spinal cord and canal). One preferred embodiment includes a system with two neuromodulators:

(A) two spinal cord stimulation (SCS) electrode arrays: one SCS electrode array configured for implantation as taught for appetite suppression (for inducing satiety) and a second SCS electrode array for placement in communication with nerve roots supplying at least one of the splanchnic nerves.

(B) two linear catheter cylindrical electrode (LCCE) arrays: one LCCE electrode array configured for implantation as taught for appetite suppression (for inducing satiety) and a second LCCE electrode array for placement in communication with nerve roots supplying at least one of the splanchnic nerves. A LCCE electrode array may be configured to have similar dimensions as a SCS electrode array, or I may have a longer electrode spacing and length, enabling a single array (of 2, 4, 8, 16, 32) channels to span multiple vertebral segments (with an array length of at least 5 cm, 10 cm, 15 cm, 20 cm, and 30 cm), allowing for greater flexibility in recruiting appropriate sympathetic targets during intraoperative testing or screening as well as for postoperative fine tuning and maintenance, including compensation for electrode migration. A LCCE electrode array may also have much narrower dimensions than present SCS arrays, including diameters of less than 1 mm, 0.75 mm. 0.5 mm, 0.25 mm).

(C) two linear catheter cylindrical electrode (LCCE) arrays: one LCCE electrode array configured for implantation as taught for appetite suppression (for inducing satiety) and a second LCCE electrode array for placement in communication with at least one portion of the splanchnic nerves. The second LCCE electrode array may be placed using fluoroscopic guidance, or with biplanar or uniplanar X-Ray machines, or using computed tomography (CT), or magnetic resonance imaging (MRI) for localization. A series of dilators may be used to create a minimally invasive access trajectory to the splanchnic nerve.

(D) one linear catheter cylindrical electrode (LCCE) array and one nerve electrode array: one LCCE electrode array configured for implantation as taught for appetite suppression (for inducing satiety) and a nerve electrode array for placement in communication one portion of the splanchnic nerves. The nerve electrode array may be at least one of several designs without departing from the present invention, including but not limited to nerve cuff, linear catheter array, helical electrode array, intraneural electrode, epineural electrode, or other design.

Renal Nerve Stimulation: Renal nerve stimulation as taught in the present invention may be used to control blood pressure, catecholamines, and electrolytes. By delivering electrical stimulation, such as with frequencies greater than 30 Hz, and preferably greater than 50 H or 100 Hz or 300 Hz or 1000 Hz, renal nerve activity is suppressed, reducing sympathetic innervation to the renal influence on blood pressure including filtration rates as well as hormonal mechanisms, including but not limited to angiotensin, aldosterone, cortisol, catecholamines, and others. Another approach to reduction of renal nerve activity is temporary or permanent nerve ablation. A disadvantage of ablative approaches is attenuation of the normal physiological ability to maintain blood pressure to the brain during movement among a variety of postural positions. For example, as a person arises from lying flat to a standing position, the sympathetic nervous system provides some activation to maintain acceptable cerebral blood pressure. If the renal nerve is ablated, this adaptive mechanism is compromised. The use of closed-loop neuromodulation, as taught in the present invention, overcomes this limitation. A closed-loop controller is taught which uses as inputs, blood pressure, heart rate, tissue oxygenation levels, body position (flat, sitting, standing, torso angle, head angle, and other measurements) and employs a control law (such as a linear control law, proportional control law, nonlinear control law, adaptive control law), and uses the measured physiological parameters as an input to a feedback driven control law and which uses body position as an input to a feedforward control law, enables such a controller to anticipate changes in actual blood pressure and requirements of blood pressure resulting from positional changes and to respond to alterations in blood pressure or deviations in blood pressure from the desired values using feedback. Similar control of pulse and tissue oxygenation may be performed using feedforward and feedback control of these parameters, separately or in conjunction with control of blood pressure and/or other parameters.

Pulmonary Nerve Stimulation: Use of closed-loop neuromodulation for the control of asthma is taught in the present invention. By delivering electrical stimulation, such as with frequencies greater than 30 Hz, and preferably greater than 50 H or 100 Hz or 300 Hz or 1000 Hz, innervation of the bronchial tree musculature is suppressed, reducing the muscle tone within the bronchial tree, including the trachea, bronchi, and distal branches. Another approach is temporary or permanent nerve ablation. A closed-loop controller is taught which uses as inputs from at least one sensor, including but not limited to sensors which transducer at least one of blood oxygenation, tissue oxygenation, pulmonary blood flow, respiratory rate (breathing rate), airway pressure, airway flow rates, sound from airway flow, vibration from airway flow, heart rate, tissue, body position (flat, sitting, standing, torso angle, head angle, and other measurements) and employs a control law (such as a linear control law, proportional control law, nonlinear control law, adaptive control law), and uses the measured physiological parameters as an input to a feedback driven control law and which may employ user input and physiological parameters as an input to a feedforward control law, enables such a controller to anticipate changes in actual airway resistance and requirements of oxygen delivery suggested from user input, breathing rates, and other physiological parameters listed herein and to respond to alterations in airway resistance or deviations in airway resistance from the desired values using feedback.

The symptoms of an asthmatic attack may be sensed by changes in air flow in the bronchial tree, including but not limited to changes in turbulence, change in laminar flow, change in air velocity, change in air pressure, change in total air flow, change in pressure gradient, change in airway resistance, and other changes. The symptoms of an asthmatic attack may be sensed by changes blood oxygenation, tissue oxygenation, by user input, by input from a physician or family member or another individual, and by other senses parameters.

Temporally Optimized Stimulation Parameter Selection

As taught in the present invention, temporal patterning of neuromodulatory signal (NMS) may be performed in a spectrum of potential methods and using a variety of potential apparati. Intermittent stimulation may be delivered based upon a pattern which may comprise a singularity, multiplicity, or combination thereof of patterns comprising fixed temporal pattern or a variable temporal pattern or a random temporal pattern. Temporal patterning may comprise modulation which is a function of prandial activity; such patterning may comprise at least one of preprandial modulation, prandial modulation, postprandial modulation, modulation in between mealtimes, daytime modulation, nighttime modulation, other modulation, or a combination thereof.

For a system in which the therapeutic modality is appetite, various patterns may be employed to deliver optimal therapy, comprising preprandial, prandial, interprandial, postprandial, baseline, daytime, nighttime, or other time window.

For a system in which the therapeutic modality is metabolic rate, various patterns may be employed to deliver optimal therapy, comprising preprandial, prandial, interprandial, postprandial, baseline, daytime, nighttime, or other time window. Various sensors may be employed to sense status relative to prandial or to glucose state, including physiologic sensors, vital sign sensors, temperature sensors, heart rate sensors, blood pressure sensors, heart rate variability sensors, body position sensors, and user input sensors, and other sensors. Sensors which sense activity of the muscles of mastication, jaw movement, masseter activity, temporalis activity, sounds generated from chewing, vibrations generated from chewing, bolus passage in the gastrointestinal tract, and other sensors may be employed. These sensors may be configured to be implanted or non-implanted or noninvasive and may be swallowed, worn, carried, or be remote from the user.

For a system in which the therapeutic modality is at least one of glucose metabolic pathway and glucose metabolism, various patterns may be employed to deliver optimal therapy, comprising preprandial, prandial, interprandial, postprandial, baseline, daytime, nighttime, or other time window.

Temporal patterning of modulation provides benefits comprising but not limited to tachyphylaxis minimization and safety, including but not limited to sympathetic drive restriction.

Specific Features for Optimization of Stimulation for Safety

For therapeutic modalities such as metabolic rate modulation, careful control of the magnitude of sympathetic stimulation and the sympathetic index are advantageous to minimize potential adverse effects from excessive sympathetic activation. Such side effects or adverse effects may comprise but are not limited to elevations in blood pressure within the normal range as well as outside the normal range (hypertension), elevations in heart rate within the normal range as well as outside the normal range (tachycardia).

One preferred embodiment comprises a system which limits aspects of Neuromodulatory Signal (NMS) to maintain safety, said limits including but not limited to amplitude, dusty cycle, area under the curve per unit time. These limits are characterized in at least one of an open loop and a closed loop manner, with preset limits, dynamic limits, or constraints which are a function of other parameters, including neural state variables, physiological parameters, including neural activity, blood pressure, heart rate, heart rate variability, renal blood flow, creatinine level, sodium level, potassium level, pH, and other physiological parameter.

Furthermore, such side effects or adverse effects may comprise but are not limited to vasoconstriction within the normal range as well as outside the normal range, which may result in relative or absolute ischemia or hypoxia and which may be systemic, focal, or involve a singularity or multiplicity of organs or components of the central circulation, peripheral circulation, cardiac circulation, cerebral circulation, mesenteric circulation, renal circulation, or other component of the circulatory system. Careful monitoring of effects of neuromodulation on relevant regions of the circulatory system may provide advantageous levels of safety not provided by known systems available today. One object of the present invention is the teaching of methods and corresponding apparatus which facilitate the delivery of safe neuromodulation which is designed and patterned to be intrinsically safe as an open-loop system and to perform additional monitoring and control to further insure safety as a closed-loop system. Such monitoring comprises but is not limited to periodic monitoring of metrics of renal function, including blood urea nitrogen (BUN), creatinine, and electrolytes, including but not limited to sodium, potassium, chloride, bicarbonate, and others. This safety monitoring may be performed by implanted sensors and by external sensors. Furthermore, said safety monitoring may be performed through monitoring by other tests comprising a blood draw and other including laboratory tests, which can be performed by the patient, by a nurse, by a physician, or by other health care professional. This may be transmitted to the control circuit using radiofrequency signal, using infrared signal, or may be typed in or entered directly by a healthcare professional or transmitted automatically via radiofrequency, infrared, bluetooth, or other transmission signal. Such metrics sensed as part of this safety monitoring process may be used as at least of inputs, feedback, and other signals to a control law or to a safety check module. At least one of said control law and safety check module limit the effective magnitude of the neuromodulatory signal to maintain physiological parameters within at least one of their safe and normal range. Said effective magnitude is modulated, influenced, or set by stimulation parameters which comprises at least of voltage, current, pulse width, duty cycle, and temporal patterning of the neuromodulation signal (NMS).

For example, if modulation of the sympathetic nervous system causes vasoconstriction of the mesenteric or renal structures, relative or absolute ischemia may occur, thereby potentially causing tissue damage. Novel functions and apparatus taught in the present invention provide protection from stimulation which could otherwise cause such undesirable effects. Such novel functions may be implemented in at least one of a variety of methods and apparatus; one such implementation is in the form of safety parameters, which may be used to govern modulation parameters, which include but are not limited to control of neuromodulation amplitude, pulse width, stimulation frequency, burst frequency, burst duration, envelope frequency, envelope duration, duty cycle, and the maximum on-time duration, minimum on-time duration, maximum-off-time duration, minimum off-time duration, of these or other parameters, and other parameters, and which may be fixed, variable, adaptive, and adjustable. For example, stimulation may be provided for at least one of the following times, pre-prandially for D_{PrePMin} to D_{PrePMax} minutes for 0, 1, 2, 3, or more times per day, prandially for D_{PMin} to D_{PMax}, for a single or multiplicity of times during 0, 1, 2, 3, post-prandially for D_{PostPMin} to D_{PostPMax} minutes for 0, 1, 2, 3, or more times post-prandially; or inter-prandially for D_{IPMin} to D_{IPMax} minutes for 0, 1, 2, 3, 4, 5, 6, or more times during any inter-prandial period; for D_{DMin} to D_{DMax} minutes during any single or multiplicity of time or times during the day or in the evening, and for D_{NMin} to D_{NMax} minutes during any single or multiplicity of time or times during the night. D_{PrePMin}, D_{PrePMax}, D_{PMin}, D_{PMax}, D_{PostPMin}, D_{PostPMax}, D_{IPMin}, D_{IPMax}, D_{DMin}, D_{DMax}, D_{NMin}, D_{NMax}, which are described as durations in minutes may be any of a variety of durations (microseconds, milliseconds, seconds, minutes, hours, days, weeks, months, years) without departing from the present invention. The minimal durations, comprising D_{PrePMin}, D_{PMin}, D_{PostPMin}, D_{IPMin}, D_{DMin}, D_{NMin} may be 0, 1, 2, 3, 4, 5, 10, 15, 20, 30, 45, 60 seconds, any value in between, or 1, 2, 3, 4, 5, 10, 15, 30, 45, 60 90, 120 minutes, any value in between, 1, 2, 3, 4, 5, 6, 7, 8, 9, 10, 11, 12, 13, 14, 15, 16, 17, 18, 19, 20, 21 22, 23, 24 hours or any value which is lesser, greater, or in between. The maximal durations, comprising D_{PrePMax}, D_{PMax}, D_{PostPMax}, D_{IPMax}, D_{DMax}, D_{NMax} may be 0, 1, 2, 3, 4, 5, 10, 15, 20, 30, 45, 60 seconds, any value in between, or 1, 2, 3, 4, 5, 10, 15, 30, 45, 60 90, 120 minutes, any value in between, 1, 2, 3, 4, 5, 6, 7, 8, 9, 10, 11, 12, 13, 14, 15, 16, 17, 18, 19, 20, 21 22, 23, 24 hours or any value which is lesser, greater, or in between. The durations and the stimulation parameters for each of the defined time periods may be the same, different, variable, adjustable, functions of similar input variable, functions of different input variables, or controlled in other manners. For none, a singular, or plurality of these defined durations, a vector of said durations could also be used to describe a set of values for the corresponding multiplicity of intervals, i.e. D_{PrePMin}, ={vector of D_{PrePiMin}} of [D_{PreP1Min}, D_{PreP2Min}, D_{PreP3Min}, D_{PreP4Min}, D_{PrePNMin}], where N indicates the number of such intervals. Further, for a singular, or plurality of these defined durations, a matrix of said durations could be used to describe a set of values for the corresponding multiplicity of intervals, i.e.

$${D}_{PrePMin ,} = \{ array of {D}_{PreP ( i , j ) Min} \} = [ \begin{matrix}{D}_{PreP ( 1 , 1 ) Min} , & {D}_{PreP ( 2 , 1 ) Min} , & {D}_{PreP ( 3 , 1 ) Min} , & … , & {D}_{PreP ( N , 1 ) Min} ; \\ {D}_{PreP ( 1 , 2 ) Min} , & {D}_{PreP ( 2 , 2 ) Min} , & {D}_{PreP ( 3 , 2 ) Min} , & … , & {D}_{PreP ( N , 2 ) Min} ; \\ {D}_{PreP ( 1 , 3 ) Min} , & {D}_{PreP ( 2 , 3 ) Min} , & {D}_{PreP ( 3 , 3 ) Min} , & … , & {D}_{PreP ( N , 3 ) Min} ; \\ … & … & … & … & \\ {D}_{PreP ( 1 , M ) Min} , & {D}_{PreP ( 2 , M ) Min} , & {D}_{PreP ( 3 , M ) Min} , & … , & {D}_{PreP ( N , M ) Min} ;\end{matrix} ] ,$$

where (i,j) specifies the matric element (1:N, 1:M) in the N×M matrix corresponding to the index of the specified interval. Using such a structure, varying minima and maxima may be set for each of a varying number of intervals such as pre-prandial, prandial post-prandial, inter-prandial, day, evening, night intervals. This matrix structure is exemplary for and representative of that which is also taught for all durations and their respective parameters and durations, safety parameters, and the minima and maxima thereof.

For example, one such preferred embodiment regimen may comprise:

(1) stimulation for a single or multiplicity of durations including 15 minutes (or any duration from D_{DiMin} to D_{DiMax}) upon awakening or thereafter as determined by user input, predetermined or sensed or timed circadian clock, chronological clock, or sensed by change in body position or body angle or by physiological parameter change and in which the subscript “i” could be a single or a vector of values;

(2a) stimulation for a single or multiplicity of pre-prandial durations including for 30 minutes (or any duration from D_{PrePMin} to D_{PrePMax}) prior to breakfast;

(2b) stimulation for a single or multiplicity of prandial durations including for 10 minutes (or any duration from D_{PMin} to D_{PMax}) during breakfast;

(2c) stimulation for a single or multiplicity of prandial durations including for 15 minutes (or any duration from D_{PostMin} to D_{PostMax}) after breakfast;

(3) stimulation for a single or multiplicity of durations including 45 minutes (or any duration from D_{IPMin} to D_{IPMax}) between breakfast and lunch;

(4a) stimulation for a single or multiplicity of pre-prandial durations including for 30 minutes (or any duration from D_{PrePMin} to D_{PrePMax}) prior to lunch;

(4b) stimulation for a single or multiplicity of prandial durations including for 10 minutes (or any duration from D_{PMin} to D_{PMax}) during lunch;

(4c) stimulation for a single or multiplicity of prandial durations including for 15 minutes (or any duration from D_{PostMin} to D_{PostMax}) after lunch;

(5) stimulation for a single or multiplicity of durations including 45 minutes (or any duration from D_{IPMin} to D_{IPMax}) between lunch and dinner;

(6a) stimulation for a single or multiplicity of pre-prandial durations including for 30 minutes (or any duration from D_{PrePMin} to D_{PrePMax}) prior to dinner;

(6b) stimulation for a single or multiplicity of prandial durations including for 10 minutes (or any duration from D_{PMin} to D_{PMax}) during dinner;

(6c) stimulation for a single or multiplicity of prandial durations including for 15 minutes (or any duration from D_{PostMin} to D_{PostMax}) after dinner;

(7) stimulation for a single or multiplicity of durations including 45 minutes (or any duration from D_{IPMin} to D_{IPMax}) between dinner and a snack;

(8a) stimulation for a single or multiplicity of pre-prandial durations including for 30 minutes (or any duration from D_{PrePMin} to D_{PrePMax}) prior to a snack;

(8b) stimulation for a single or multiplicity of prandial durations including for 5 minutes (or any duration from D_{PMin} to D_{PMax}) during a snack;

(8c) stimulation for a single or multiplicity of prandial durations including for 15 minutes (or any duration from D_{PostMin} to D_{PostMax}) after a snack;

(9) stimulation for a single or multiplicity of durations including for 30 minutes (or any duration from D_{DMin} to D_{DMax}) during a single or plurality of daytime or evening periods;

(10) stimulation for a single or multiplicity of durations including for 30 minutes (or any duration from D_{NMin} to D_{NMax}) during a single or plurality of night time periods;

In FIG. 51, safety check module 396 is depicted, including a singularity or multiplicity of safety state sensors 397, which sense, input, or acquire signals which are transmitted to signal conditioning module 398, which conditions signals and transmits conditioned signals to signal processing module 399, which processes signals and transmits processed signals to control circuit 400, which implements a control law and which implements or is interfaced with at least one safety lockout 401, and which sends control output (U) 997 to output stage circuit 402, which at least one of regulates, amplifies, and modulates control output (U) 997 to generate neuromodulation signal (NMS) 998 which is transmitted via wired, wireless, optical, thermal, vibrotactile, ultrasound, sonic, subsonic, high frequency ultrasound (HIFU), or other modality to neuromodulator array 316, which may perform transformation of energy into the same or different energy modality and delivers neuromodulation signal (NMS) 998 to target tissue, comprising but not limited to nervous system component or other tissue or structure. Portions of FIG. 51 may comprise a subset of FIG. 38. The union of FIG. 38 and FIG. 51, each depicting more relatively detail with respect to the Neuromodulation System (NMS) 999 and the Safety Check module 396, respectively, teach one preferred embodiment of the Neuromodulation System (NMS) 999 with enhanced safety features.

Safety state sensors 397 comprise any sensor, input device, transmission device, receiving device or other element which facilitates the sensing or acquisition of information or signals representative of information which may be processed to extract information which may be used to assess, monitor, or control metrics, parameters, or neuromodulation signals (NMS) 998 which relate to or insure safety of Neurological Control system 999. Such metrics include but are not limited to those shown in FIG. 50 as well as those shown in FIG. 38.

Safety state sensors 397 comprise but are not limited to implanted sensors 403, external sensors 404, telemetered inputs 405, manual inputs 406, and other sensors, inputs, and receivers.

Implanted sensors 403 comprise but are not limited to physiological sensor array 319, metabolic sensor array 321, alimentary sensor array 322 (shown in more detail in FIG. 38), EMG sensor array 320, neural sensor array 318, and other sensors and arrays thereof.

External sensors 404 comprise but are not limited to physiological sensor array 319, metabolic sensor array 321, alimentary sensor array 322 (shown in more detail in FIG. 38), EMG sensor array 320, neural sensor array 318, nonimplanted sensors, percutaneous sensors, and other sensors and arrays thereof.

Telemetered inputs 405, comprise but are not limited to a singularity or multiplicity of modulation authorization code 407, renal function metrics 408, physiological sensor array 319, metabolic sensor array 321, alimentary sensor array 322 (shown in more detail in FIG. 38), EMG sensor array 320, neural sensor array 318, implanted sensors, nonimplanted sensors, percutaneous sensor, and other sensors and arrays thereof.

Manual inputs 406, comprise but are not limited to a singularity or multiplicity of modulation authorization code 407, renal function metrics 408, physiological sensor array 319, metabolic sensor array 321, alimentary sensor array 322 (shown in more detail in FIG. 38), implanted sensors, nonimplanted sensors, percutaneous sensor, and other sensors and arrays thereof.

In one preferred embodiment shown in FIG. 50, user is required to undergo regular therapy safety testing and therapy efficacy testing, to be performed at substantially the same intervals or at different intervals, such as daily, weekly, biweekly, monthly, or over a longer or shorter time interval. Said therapy safety testing comprises but is not limited to the tests and metrics shown in the table presented in FIG. 51.

One objective of the present invention is to closely monitor the physiology of the patient or user of Neurological Control System (NMS) 999 to facilitate early detection of potential adverse effects and to monitor physiological trends such that physiological state 408, neural state 409, and safety state 410 may be at least one of anticipated, predicted, and controlled to prevent adverse effects. For example, sympathetic activation of vascular structures may cause vasoconstriction. Vasoconstriction, if effected for prolonged periods of time may cause ischemia and necrosis if not regulated or controlled. An objective of the present invention is the close monitoring of physiological functions and metrics which provide insight into causative processes underlying adverse effects and monitoring of early signs of adverse effects.

Safety state sensors 397 comprise sensors which sense at least one of physiological state 408, neural state 409, safety state 410, and other state or signal, comprising signals from implanted sensors 403, external sensors 404, telemetered sensors 504, and manual inputs 406, and other sensors or input devices. Safety State Sensors 397 may be configured to sense at least one of any singular or plurality of signals or arrays thereof, comprising those depicted in FIG. 50, FIG. 51, and others listed, described, implied, or related to those taught in the present invention including specification and figures, the text and content of which is incorporated by reference.

Sensors comprising but not limited to those which sense states comprising but not limited to physiological state 408, neural state 409, safety state 410, are configured to sense at least one of: physiological signals, metabolic signals, modulation authorization codes, renal function metrics. Physiological state 408 comprises a singularity or plurality of at least one of vital signs (comprising heart rate, blood pressure, respiratory rate, temperature, urine output, heart rate variability, pulse oximetry, oxygen concentration in at least one component or region of the body or external support system including ventilator or tubing or component thereof.

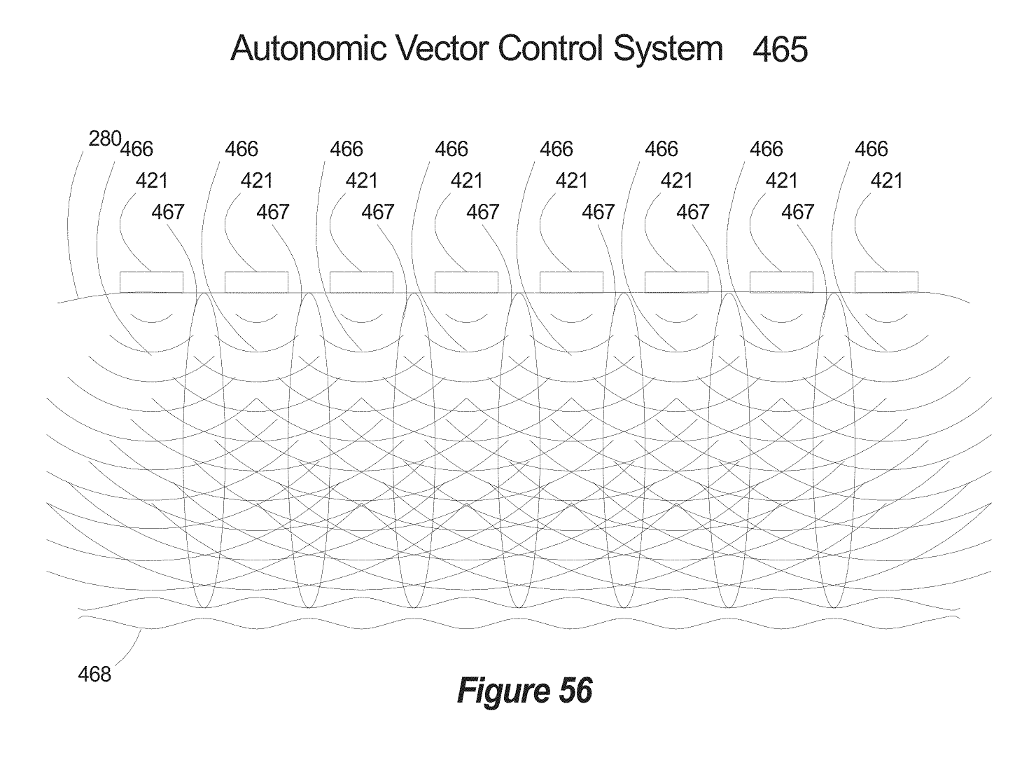

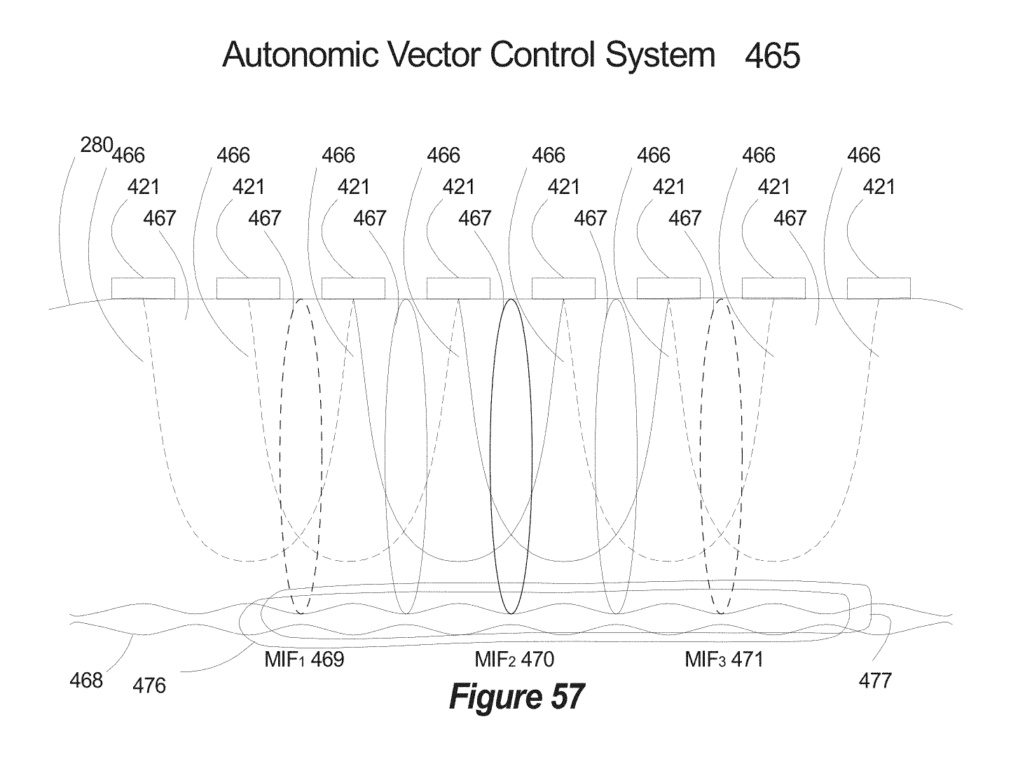

FIG. 51 apparatus, when generating at least one neuromodulatory signal (NMS) 998 and preferably a multiplicity of neuromodulatory signals (NMS) 998, and used in conjunction with novel configurations described in FIGS. 56 and 57, may be used to generate Neuromodulation Signal Vector (NSV) 996, which has numerous advantages in efficacy and in safety over scalar neuromodulatory signals.

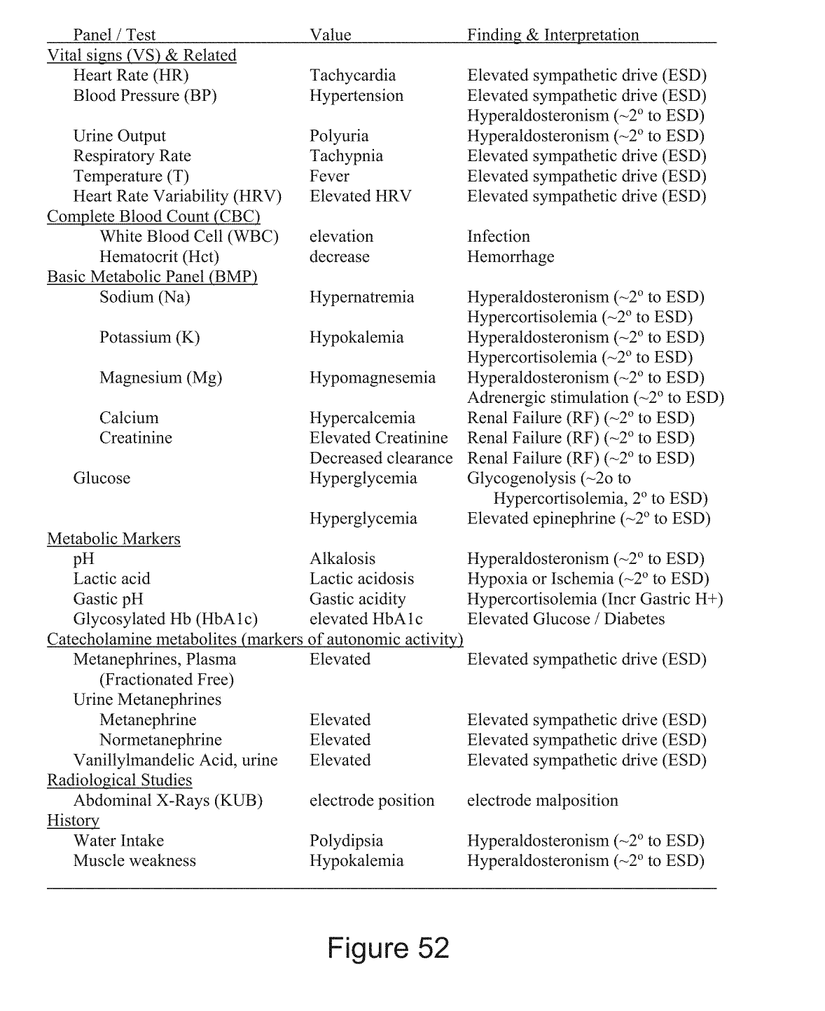

FIG. 52 presents in tabular form various biomarkers that may be used in conjunction with the present invention to calibrate the system, verify autonomic changes and quantify response to therapy.

Minimally Invasive Stimulation:

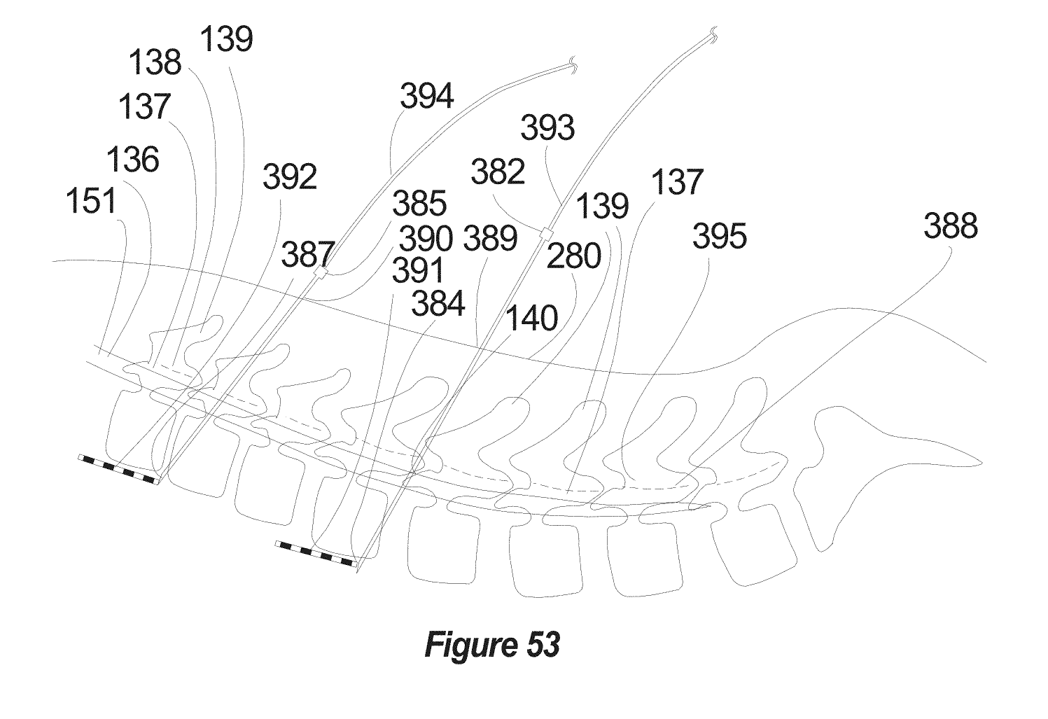

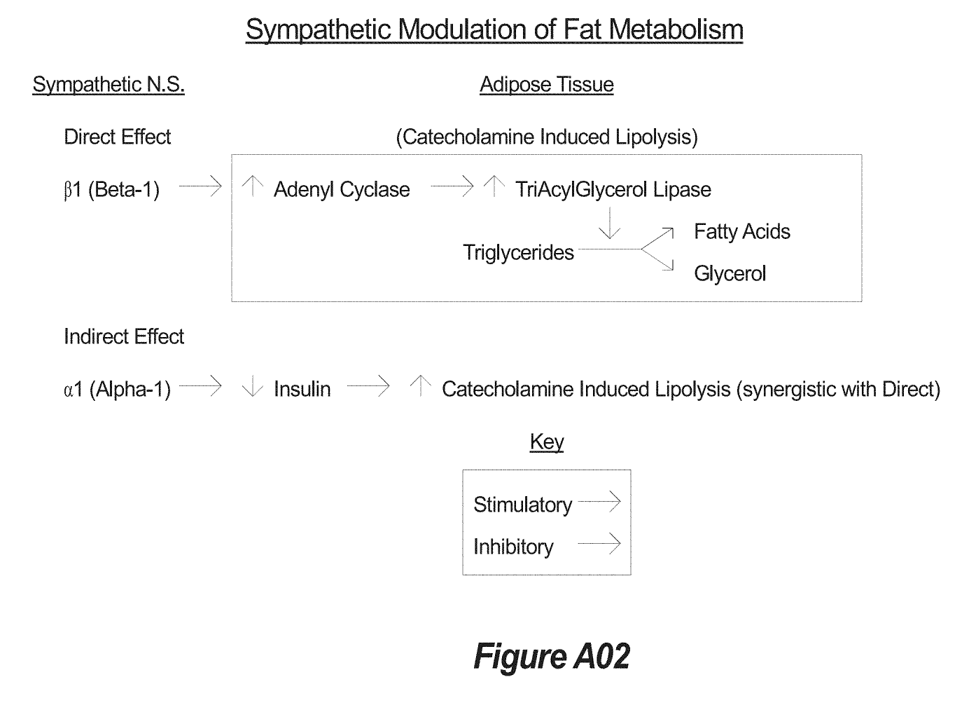

FIG. 53 depicts one preferred embodiment of a minimally-invasive modulation configuration, which may be implemented using devices with a very small profile, comprising implanted electrodes with embedded electronics and telemetering circuitry. Using this method and apparatus, a single or multiplicity of neuromodulatory interfaces 391, 392 may be implanted and which may be internally powered or externally powered and which may be internally or externally controlled. Telemetry of at least one of control signals and power signals may be implemented using radiofrequency (RF) energy, microwave energy, millimeter wave energy, sub-millimeter wave energy, direct current (DC) energy, alternating current (AC) energy, optical energy, infrared energy, near infrared energy, visible energy, ultraviolet energy, vibratory energy, sonic energy, ultrasonic energy, subsonic energy, thermal energy, chemical energy, fuel cell energy, motion energy, energy derived from physiological sources, or other forms of energy.

Power: Neuromodulatory interfaces 391, 392 may have internal energy storage elements comprising at least one of internal battery power storage, internal fuel cell power storage, internal capacitive poser storage, internal inductive power storage, or other internal power storage means. Neuromodulatory interfaces 391, 392 may have external energy storage elements comprising at least one of external battery power storage, external fuel cell power storage, external capacitive poser storage, external inductive power storage, or other external power storage means. Neuromodulatory interfaces 391, 392 may have augmented energy storage elements which may be implanted or non-implanted and which may be separate from the neuromodulatory interface and may at least one of supply, store, and relay power to neuromodulatory interfaces 391, 392.

Control signals: Control signals comprise Control Output (U), Neuromodulatory Signal (NMS), signals to or from patient interface module, signals to or from supervisory module, or signals to or from other modules. At least one of Control Output (U), Neuromodulatory Signal (NMS), other control signals, output stage internal signal, neural state (X), or other signal, may be transmitted as at least one of the power signal, an impedance drawn on the power signal, as a form of modulation of the power signal, as amplitude modulation of the power signal, as frequency modulation of the power signal, as phase modulation of the power signal, and as a separate signal, which may be implemented as radiofrequency (RF) energy, microwave energy, millimeter wave energy, sub-millimeter wave energy, direct current (DC) energy, alternating current (AC) energy, optical energy, infrared energy, near infrared energy, visible energy, ultraviolet energy, vibratory energy, sonic energy, ultrasonic energy, subsonic energy, thermal energy, chemical energy, fuel cell energy, motion energy, energy derived from physiological sources, or other forms of energy. Neuromodulatory interfaces 391, 392 may be surgically implanted via an open or minimally invasive procedure, including placement using laparoscopic, ventriculoscopic, or other endoscopic technique or they may be inserted using a catheter guide tube or be injected. They may have a tethering tail which provides a mechanical means for removal, a catheter which includes connecting cable 393, 394 with electrical wires to facilitate signal transmission from an implanted or external Neuromodulatory Signal (NMS) source, or may be wireless with active or passive electronics facilitating receipt of at least one of Neuromodulatory Signal (NMS) and power as well as transmission of signal via wireless means.

Relevant anatomy shown in FIG. 53 comprises spinal cord 151, Meningeal Layer of Dura Mater 136, Epidural Space 137, Periosteal Layer of Dura Mater 138, Vertebral Spinous Process 139, Vertebral Facet 140, and Ligamentum Flavum 388. Cannula 382 is shown piercing skin 280 at entry point 389 and with cannula tip 384 in position to introduce neuromodulatory interface 391 into the desired position; in this case it is shown adjacent to a neural target in the vicinity of the vertebral body, and other target locations are envisioned within the present invention. Cannula 385 is shown piercing skin 280 at entry point 390 and with cannula tip 387 in position to introduce neuromodulatory interface 392 into the desired position; in this case it is shown adjacent to a neural target in the vicinity of the vertebral body, and other target locations are envisioned within the present invention.

Percutaneous Stimulation:

Applications: The present invention teaches the use of percutaneous neuromodulation including at least one of stimulation and inhibition of autonomic structures and other neural and gastrointestinal structures for at least one of the characterization of metabolic conditions, the diagnosis of metabolic conditions, the trialing of metabolic conditions, Obesity, diabetes, hyperlipidemia, hypercholesterolemia and the treatment of metabolic conditions and for the treatment of any other conditions described or referred to in the this specification or others incorporated by reference.

The present invention teaches the use of percutaneous neuromodulation including at least one of stimulation and inhibition of autonomic structures and other neural and gastrointestinal structures for at least one of the characterizations of metabolic conditions, the diagnosis of metabolic conditions, the trialing of metabolic conditions, and the treatment of metabolic conditions.

Biochemical Mechanisms involved in Sympathetic Modulation of Fat Metabolism:

FIG. A02 depicts contributory biochemical mechanisms of action comprising effects of sympathetic nervous system modulation on adipose tissue and specifically its effects on fat metabolism. A direct effect involving beta-1 fiber activation is shown to activate adenyl cyclase, which activates triacylglycerol lipase, which catalyzes the breakdown of triglycerides into fatty acids and glycerol An indirect effect involving alpha-1 fiber activation is shown to decrease insulin levels, which facilitates increased catecholamine induced lipolysis (and which is synergistic with the direct effect).

Non-Invasive Stimulation:

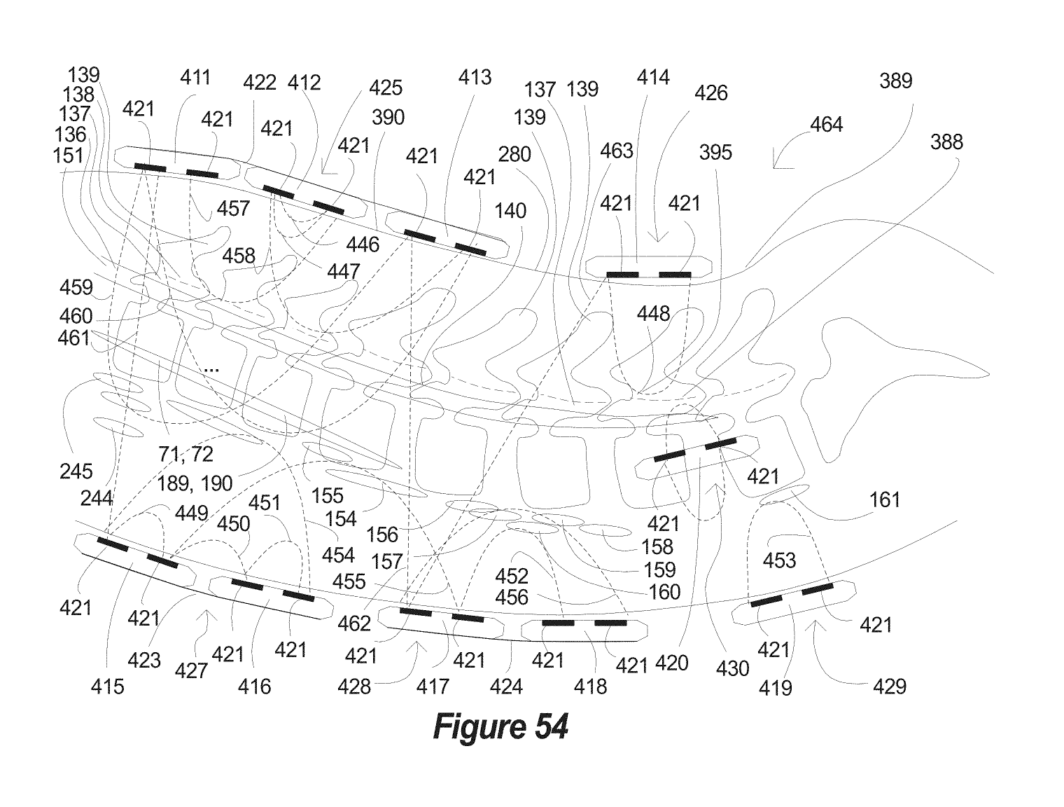

FIG. 54 demonstrates one preferred embodiment of a noninvasive neuromodulatory configuration with some relevant anatomy. Neuromodulator assemblies of neuromodulator belts are shown in a variety of exemplary configurations; other configurations may be implemented without departing from the present invention. This figure demonstrates field lines which for modulation using energy forms including but not limited to electrical, magnetic, optical, subsonic, sonic, ultrasound, high frequency ultrasound, thermal, vibratory, and others.

Multiple neuromodulator configurations are depicted, and more or fewer or a singularity or multiplicity are included in the present invention. Neuromodulator groups 425, 426, 427, 428, 429 are shown in proximity to skin 280. Any of neuromodulator groups 425, 426, 427, 428, 429 may be at least one of in contact with, in close proximity to, or distant from skin 280, without departing from the present invention. Further, any of neuromodulator groups 425, 426, 427, 428, 429 could be configured with percutaneous elements, including electrodes or other energy delivery elements, or be implanted, without departing from the present invention. Further, each of neuromodulator groups 425, 426, 427, 428, 429 may comprise a at least one of a single or a plurality of neuromodulator arrays, comprising neuromodulator array 411, 412, 413, 414, 415, 416, 417, 418, 419, 420, and a single or plurality of neuromodulator 421. Energy delivery elements include but are not limited to macroelectrodes, microelectrodes, percutaneous electrodes, magnetic coils, microfabricated electrodes, microfabricated coils, optical fibers, ultrasound transducers, sonic transducers, auditory transducers, speakers, vibratory transducers, piezoelectric transducers, solenoids, electromechanical transducers, mechanical transducers, pressure transducers, thermal transducers, heat sources, cold sources, chemical sources, pharmaceutical sources, biochemical sources, and biochemical compound reservoir,

Energy delivery elements further include but are not limited to biochemical compound simulator, molecular signal transducer, low frequency molecular signal transducer, high frequency molecular signal transducer, molecular signal transducer electrode, molecular signal transducer magnetic coil.

Neuromodulator group 425 is shown comprising Neuromodulator Band 422 which provides at least one of mechanical and electrical connection between Neuromodulator Array 411, 412, 413 each of which comprise arrays of a single or plurality of Neuromodulator 421.

Neuromodulator group 426 is shown comprising Neuromodulator Array 414 and may comprise a single or plurality of at least one of Neuromodulator Array 414 and neuromodulator 421. Neuromodulator Array 414 is shown comprising a plurality of Neuromodulator 421 and may comprise a single or plurality of Neuromodulator 421.

Neuromodulator group 427 is shown comprising Neuromodulator Band 423 which provides at least one of mechanical and electrical connection between Neuromodulator Array 415 and 416, each of which comprise arrays of a single or plurality of Neuromodulator 421. Neuromodulator group 427 may comprise additional or fewer instances of neuromodulator array 415, 416, and additional or fewer instances of neuromodulator 421 without departing from the present invention.

Neuromodulator group 428 is shown comprising Neuromodulator Band 424 which provides at least one of mechanical and electrical connection between Neuromodulator Array 417 and 418, each of which comprise arrays of a single or plurality of Neuromodulator 421. Neuromodulator group 427 may comprise additional or fewer instances of neuromodulator array 417, 418, and additional or fewer instances of neuromodulator 421 without departing from the present invention.

Neuromodulator group 429 is shown comprising Neuromodulator Array 419 and may comprise a single or plurality of at least one of Neuromodulator Array 419 and neuromodulator 421. Neuromodulator Array 419 is shown comprising a plurality of Neuromodulator 421 and may comprise a single or plurality of Neuromodulator 421. Neuromodulator group 429 could further include a Neuromodulator Band 422, 423, 424, and which provides at least one of mechanical and electrical connection between Neuromodulator Array 419 additional instances of neuromodulator array and neuromodulator 421. Neuromodulator group 429 may comprise additional or fewer instances of neuromodulator array 419, and additional or fewer instances of neuromodulator 421 without departing from the present invention.

Neuromodulator group 430 is shown comprising Neuromodulator Array 420 and may comprise a single or plurality of at least one of Neuromodulator Array 420 and neuromodulator 421. Neuromodulator Array 420 is shown comprising a plurality of Neuromodulator 421 and may comprise a single or plurality of Neuromodulator 421. Neuromodulator group 430 could further include a Neuromodulator Band 422, 423, 424, and which provides at least one of mechanical and electrical connection between Neuromodulator Array 420 additional instances of neuromodulator array and neuromodulator 421. Neuromodulator group 430 may comprise additional or fewer instances of neuromodulator array 420, and additional or fewer instances of neuromodulator 421 without departing from the present invention.

Neuromodulator group 430, shown comprising array 420, is shown within the confines of the outer surface of skin 280; tis may be inserted surgically, through minimally invasive technique, through natural orifices, through ingestion such as by swallowing by the patient, through placement per os (PO) including use of an endoscope or other device or procedure, through placement per rectum (PR) including placement manually or through use of an endoscope or other device or procedure, or other technique for placement within the confines of the body. Using this technique, Neuromodulator group 430 may remain in the intraluminal space or be inserted transluminally into another space. Neuromodulator group 430 may be placed within the hollow visceral organs including any segment or portion of the gastrointestinal tract. Neuromodulator group 430 may be inserted through the hollow visceral organs including any segment or portion of the gastrointestinal tract and placed in another space, such as within the cervical region, thoracic region, thoracic cavity, peritoneal region, peritoneal cavity, pelvic region, pelvic cavity, adjacent to or affixed to the prevertebral fascia, adjacent to or affixed to the esophagus, adjacent to or affixed to the trachea, adjacent to or affixed to the mediastinum, adjacent to or affixed to the aorta, adjacent to or affixed to the vena cava, adjacent to or affixed to an artery, adjacent to or affixed to a vein, adjacent to or affixed to a nerve, adjacent to or affixed to a ganglion, adjacent to or affixed to a plexus, adjacent to or affixed to an organ, adjacent to or affixed to a neural target, adjacent to or affixed to a physiological target, adjacent to or affixed to an adrenal gland, adjacent to or affixed to a kidney, adjacent to or affixed to the stomach, adjacent to or affixed to the pylorus, adjacent to or affixed to a celiac plexus, adjacent to or affixed to a mesenteric plexus, adjacent to or affixed to a renal nerve, adjacent to or affixed to another target.

Neuromodulator group 430, shown comprising array 420, is shown within the confines of the outer surface of skin 280; tis may be inserted surgically, through minimally invasive technique, through vascular structures, including use of an endovascular technique or device or procedure, or other technique for placement within the confines of the body. Using this technique, Neuromodulator group 430 may remain in the intravascular space or be inserted transvascularly into another space. Neuromodulator group 430 may be placed within the hollow visceral organs including any segment or portion of the gastrointestinal tract. Neuromodulator group 430 may be inserted through the hollow visceral organs including any segment or portion of the gastrointestinal tract and placed in another space, such as within the cervical region, thoracic region, thoracic cavity, peritoneal region, peritoneal cavity, pelvic region, pelvic cavity, adjacent to or affixed to the prevertebral fascia, adjacent to or affixed to the esophagus, adjacent to or affixed to the trachea, adjacent to or affixed to the mediastinum, adjacent to or affixed to the aorta, adjacent to or affixed to the vena cava, adjacent to or affixed to an artery, adjacent to or affixed to a vein, adjacent to or affixed to a nerve, adjacent to or affixed to a ganglion, adjacent to or affixed to a plexus, adjacent to or affixed to an organ, adjacent to or affixed to a neural target, adjacent to or affixed to a physiological target, adjacent to or affixed to an adrenal gland, adjacent to or affixed to a kidney, adjacent to or affixed to the stomach, adjacent to or affixed to the pylorus, adjacent to or affixed to a celiac plexus, adjacent to or affixed to a mesenteric plexus, adjacent to or affixed to a renal nerve, adjacent to or affixed to another target.

Neuromodulation Field Alignment

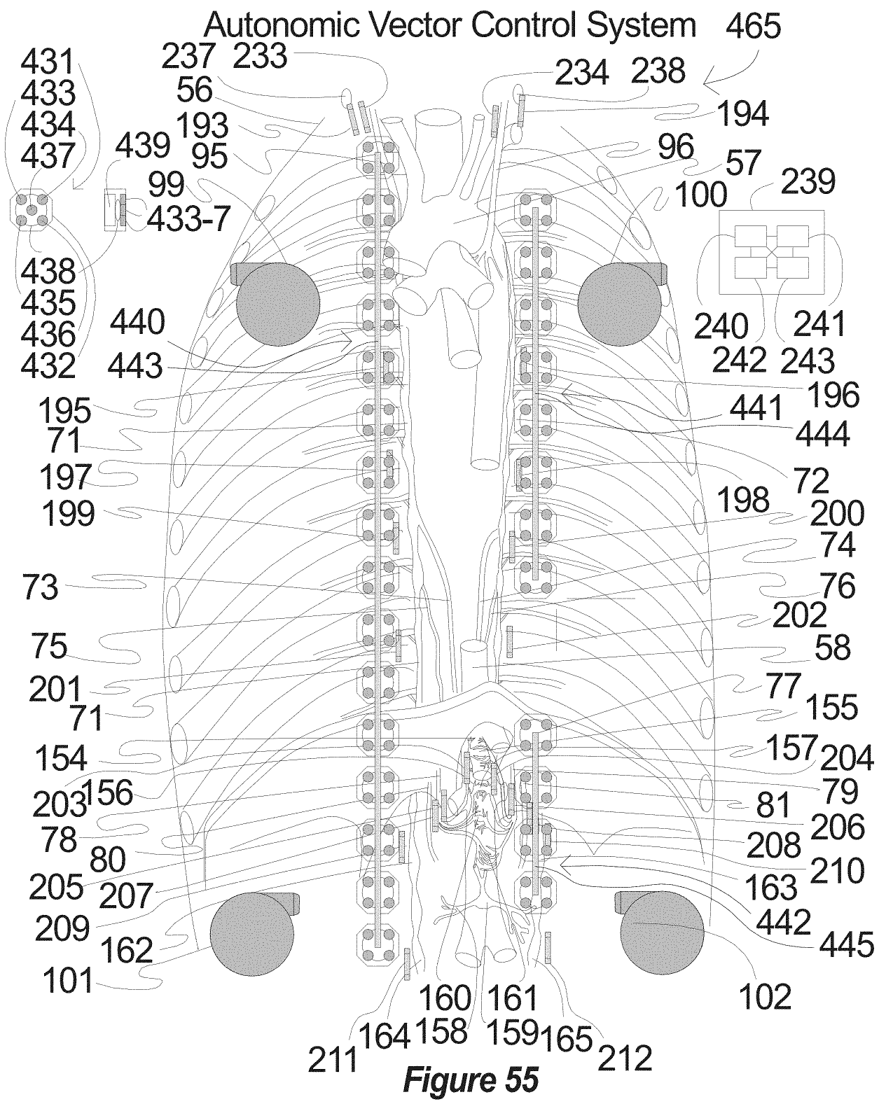

FIG. 55 demonstrates one preferred embodiment of Autonomic Vector Control System (AVCS) 465 with some relevant anatomy. Multiple modalities are taught and exemplified comprising an autonomic vector control system. Implantable Pulse Generator 99, 100, 101, 102 are shown; these may be implantable, nonimplanted, external, worn as part of clothing, worn as a wearable device, or implemented in any other manner without departing form the present invention.

Multimodal Surface Neuromodulator (MSM) 431 is shown with components comprising MSM Outer Surface 432, MSM Modulator Element A 433, MSM Modulator Element B 434, MSM Modulator Element C 435, MSM Modulator Element D 436, MSM Modulator Element E 437, MSM Modulator Coil 438, and MSM Power and Control Electronics 439. Multimodal Surface Neuromodulator (MSM) 431 may comprise additional of fewer MSM Modulator elements and other elements or components without departing from the present invention. Each of MSM Outer Surface 432, MSM Modulator Element A 433, MSM Modulator Element B 434, MSM Modulator Element C 435, MSM Modulator Element D 436, and MSM Modulator Element E 437 may each deliver a modulatory signal of the same or of different modulatory modalities.

Said modulatory modalities include but are not limited to noninvasive modulation and invasive modulation, modulation comprising at least one of stimulation, inhibition, activation, and blocking, modulation including tactile stimulation, including light touch, pressure, vibration, thermal stimulation, including hot and cold, as well as constant or variable temperatures, electrical stimulation, including direct current stimulation, alternating current stimulation, interferential stimulation, magnetic stimulation, including transcranial magnetic stimulation (TMS), transcutaneous magnetic stimulation, implanted magnetic stimulation, electromagnetic stimulation, including stimulation with radiofrequency energy (including but not limited to extremely low frequency (ELF) 3-30 Hz, super low frequency (SLF) 30-300 Hz, ultra low frequency (ULF) 300-3000 Hz, very low frequency (VLF) 3-30 kHz, low frequency (LF) 30-300 kHz, medium frequency (MF) 300 kHz-3 MHz, high frequency (HF) 3-30 MHz, very high frequency (VHF) 30-300 MHz, ultra-high frequency (UHF) 300 MHz-3 GHz, super high frequency (SHF) 3-30 GHz, extremely high frequency (EHF) 30-300 GHz, tremendously high frequency (THF) 300 GHz-3 THz, microwave bands, millimeter wave bands, and other bands), and other electromagnetic frequency bands, optical stimulation, including infrared light (IR) 300 GHz-430 THz (including far-infrared (FIR) 300 GHz-30 THz, mid-infrared (MIR) 30 THz-120 THz, and near infrared (NIR) 120 THz-400 THz bands), visible light 400-789 THz (including red 400-484 THz, orange 484-508 THz, yellow 508-526 THz, green 526-606 THz, blue 606-668 THz, violet 668-789 THz), ultraviolet light (UV) 400 nm-100 nm (including ultraviolet A (UV-A) 400 nm-315 nm, ultraviolet B (UV-B) 315 nm-280 nm, and ultraviolet C (UV-C) 280 nm-200 nm, and vacuum UV (UV-V) 200 nm-100 nm), X-rays, gamma rays, and other stimulation with photons of these and other wavelengths, pressure wave stimulation including ultrasonic, high frequency ultrasonic, sonic, subsonic, and other pressure wave frequencies, chemical stimulation, stimulation using pharmacological agents, stimulation using electromagnetic signals which emulate signals generated by chemical and biological compounds, and other modalities which control, modulate, or influence neural activity, neural signals, or other physiological activity.

Neuromodulator Group 440, Neuromodulator Group, 441, Neuromodulator Group, 442, Neuromodulator Band, 443, Neuromodulator Band 444, Neuromodulator Band 445 are shown; additional or fewer instances of each of these may comprise the Autonomic Vector Control System (AVCS) 465 without departing from the present invention.

Many of the other components of Autonomic Vector Control System (AVCS) 465 shown in FIG. 55 are also described in FIGS. 28, 29, 30, 31, 32, and 46.

Autonomic Vector Control (AVC) as taught in the present invention is novel and useful in many respects and enables treatment efficacy and safety not otherwise possible. Built upon the novel construct of autonomic nodes, three modalities of progressively increasing sophistication and dimensionality are enabled by the present invention: (1) Autonomic Node Modulation, (2) Autonomic Vector Modulation and (2) Autonomic Matrix Modulation.

Autonomic Node Construct:

Heretofore, the autonomic nervous system has been understood and described as a one-dimensional entity with two linearly opposing components, the sympathetic and the parasympathetic nervous systems. Congruent with this understanding, the autonomic nervous system has been described as simplistic balance between two activities, a “fight and flight” sympathetic response and a “rest and digest” parasympathetic response. This simplistic view has remained unchanged over the decades. A novel aspect of the present invention is based upon the innovative construct in which the autonomic nervous system can be more precisely modeled as a multivariable vector of autonomic nodes. Applying this innovative model in the design of a novel therapy has made possible the design of the present invention which facilitates the surprising but important innovations, including modulation of specific nodes of the autonomic nervous system and the precise control of specific physiological functions controlled by respective autonomic nodes and combinations thereof.

In this construct, nodes may be defined as components segmented by spinal level, by target organ, by physiological function, by pathway including autonomic pathway and other pathways (sympathetic, parasympathetic, enteric, somatosensory, motor, visual, auditory, and other pathway, including but not limited to pathways listed elsewhere in the present application), by neurotransmitter (glutamine, acetylcholine, glycine, GABA, dopamine, serotonin, norepinephrine (noradrenaline), histamine, adenosine, adenosine triphosphate (ATP), and others, including but not limited to neurotransmitters listed elsewhere in the present application), by receptor (cholinergic, nicotinic, muscarinic, dopaminergic (including but not limited to D1 and D2 receptors), serotonergic, and others, including but not limited to receptors listed elsewhere in the present application), and by other dimensions of distinction.

In addition to these autonomic dimensions of distinction, the autonomic nervous system network comprises a multiplicity of neural pathways each of which potentially represent a distinct anatomical pathway element within a corresponding vector of anatomically distinct nodes along an anatomical pathway dimension. This may be orthogonal to or have some dependence upon a vector comprising anatomical spinal levels. This construct provides the foundation for a high dimensionality model for autonomic physiology and which facilitates the design, discovery, characterization, and validation of novel therapies which have far more efficacy and safety than present pharmacological and neuromodulatory therapies. Present pharmacotherapies are restricted only by the dimension of receptor specificity and therefore have limited anatomical specificity and often have dose limiting side effects. Present neuromodulatory therapies are generally restricted by nerve and therefore have limited pathway specificity. By employing a high dimensionality model and framework, a greater degree of modulatory specificity is achieved, permitting simultaneously greater efficacy and safety and in doing so, enabling the creation of novel therapies previously not possible or not sufficiently safe to be feasible.

A further dimension for assessment and for modulation of the autonomic vector is characterizable according to cranial level and spinal level, including but not limited to cranial nerves CN1, CN2, CN3, CN4, CN5, CN6, CN7, CN8, CN9, CN10, CN11, CN12, cervical spinal levels, C1, C2, C3, C4, C5, C6, C7, C8, thoracic spinal levels T1, T2, T3, T4, T5, T6, T7, T8, T9 T10, T1, T12, lumbar spinal levels L1, L2, L3, L4, L5, sacral spinal levels S1, S2, S3, S4, S5, and coccygeal spinal level Cxl. These spinal levels may be further subdivided by modality, or modalities may comprise a separate dimension or multiplicity of dimensions.

A further dimension which is anatomically orthogonal to the spinal levels is the dimension along the proximal-distal axis along the neural pathways, herein termed proximality-distality. For the sympathetic nervous system, the elements include but are not limited to the hypothalamus, locus ceruleus, intermediolateral nucleus, spinal nerve, dorsal root ganglion, paravertebral ganglia (including but not limited to the stellate ganglia, sympathetic trunk ganglia, lumbar ganglia), sympathetic trunk, sympathetic white rami, sympathetic grey rami, splanchnic nerves, prevertebral ganglia (including celiac ganglia, superior mesenteric ganglia, inferior mesenteric ganglia) and effector organs. For the parasympathetic nervous system, the elements include but are not limited to the hypothalamus, solitary nucleus, Edinger-Westphal nucleus, superior salivary nucleus, inferior salivary nucleus; preganglionic nerves: oculomotor nerve (parasympathetic roots), facial nerve (greater petrosal nerve), facial nerve (chorda tympani), glossopharyngeal nerve (lesser petrosal nerve), vagus nerves, pelvic nerves; paired head and neck ganglia (including but not limited to the ciliary ganglia, pterygopalatine ganglia, submandibular ganglia, and otic ganglia); postganglionic nerves: short ciliary nerves, nasopalatine nerve, pterygopalatine nerve, greater palatine nerve, lesser palatine nerve, auriculotemporal nerve, lingual nerve, pelvic splanchnic nerve, inferior hypogastric plexus.

Characterizing the autonomic nervous system (including the sympathetic nervous system and parasympathetic nervous system) and the enteric nervous system as a multidimensional matrix and further characterizing efficacy and side effects associated with each autonomic node allows for true optimization of neuromodulation performance, realizing maxima of efficacy and minima of side effects. This facilitates a dramatically more precise and granular conceptualization of autonomic physiology, and enables the informed design, development, validation, and characterization of a next generation of neuromodulation-based therapies, including but not limited to Autonomic Node Modulation 480, Autonomic Vector Modulation 472, and Autonomic Matrix Modulation 479, described below.

Autonomic Node Modulation:

The generation and delivery of the neuromodulatory vector taught in the present invention facilitates precise modulation of individual autonomic nodes as well as groups of autonomic nodes, and the neuromodulatory vector further facilitates independent modulation of at least one of individual nodes and groups of autonomic nodes with a single or a multiplicity of independent neuromodulatory signals comprising the neuromodulatory vector.

FIG. 56 and in FIG. 57 depict embodiments of the present invention, which comprises apparatus and methods in which fields, including but not limited to energy fields and interferential energy fields, are used to perform autonomic node modulation.

Autonomic Nodal Control for Autonomic Vector Modulation:

Each of Multimodal Surface Neuromodulator (MSM) 431 possesses the capability to deliver at least one modulatory modality at a focal location, including but not limited to a node within the autonomic nervous system. The novel configuration taught in the present invention comprises an array of Multimodal Surface Neuromodulator (MSM) 431 configured to concurrently modulate a vector of autonomic nodes.

One application of this is in the treatment of obesity in which one representative autonomic neuromodulatory vector increases metabolic rate while concurrently decreasing or stabilizing at least one of heart rate and blood pressure. This novel construct and combination of innovative apparatus and methods allows for maximization and/or optimization of efficacy in weight loss as well as minimization and/or optimization of side effects including possible tachycardia and hypertension as well as others.

Autonomic Nodal Differential Modality Control for Autonomic Matrix Modulation:

Each of Multimodal Surface Neuromodulator (MSM) 431 possesses the capability to deliver multiple modalities at the same location. Through the use of a single or multiplicity of modulatory modalities, each Multimodal Surface Neuromodulator (MSM) 431 may stimulate, inhibit, activate, block, or modulate at least one component of the autonomic nervous system at that node.

In some anatomical locations, furthermore, differential modulation of distinct autonomic and other neural pathways is facilitated. For example, this may involve one Multimodal Surface Neuromodulator (MSM) 431 configured to deliver an electrical modulatory signal which activates sympathetic nervous system fibers and an ultrasound modulatory signal which inhibits parasympathetic fibers. Alternatively or concurrently, at least one Multimodal Surface Neuromodulator (MSM) 431 could be configured to deliver a sonic modulatory signal to stimulate sympathetic nervous system components in a node and to inhibit sympathetic nervous system pathways in the same or adjacent regions and which may comprise the same node or distinct nodes.

Neuromodulator assemblies of neuromodulator belts are shown in a variety of exemplary configurations; other configurations may be implemented without departing from the present invention. This figure demonstrates field lines which for modulation using energy forms including but not limited to electrical, magnetic, optical, subsonic, sonic, ultrasound, high frequency ultrasound, thermal, vibratory, and others.

Optimization of ligand specific signal generation

A further dimension taught in the present invention is the use of electrical, magnetic, electromagnetic, and other signals which mimic the signals produced by the electron cloud of neurotransmitters, and other atoms and molecules and which, when used in conjunction with the novel autonomic vector modulation taught in the present invention, provides a further dimension of modulation specificity. This additional dimension allows for even greater selective modulation for optimization of efficacy, for optimization of side effects and side effect profile, and for simultaneous optimization of efficacy and side effects.

For example, by delivering a signal which mimics stimulatory effects of the preganglionic neurotransmitter at the autonomic ganglion or of the postganglionic neurotransmitter at the end effector organ or any other neurotransmitter in the autonomic matrix, the autonomic end organ may be stimulated. By concurrently or simultaneously or in a temporally patterned manner preceding or following previously said stimulation, delivering a signal which mimics an inhibitory neurotransmitter or blocks an activating neurotransmitter in the autonomic matrix, a very high degree of specificity in modulation is realized; and this facilitates optimization and maximization of efficacy, optimization and minimization of side effects, and concurrent optimization of efficacy and side effects.

Said energy signals (including electric fields, magnetic fields, electromagnetic field, and other energy fields) which may mimic neurotransmitters may be in any frequency range, including those ranges listed elsewhere in this specification and may include activation stimulation and/or blockage or inhibition of autonomic (or somatic, enteric, or other) pathways or a single or plurality of autonomic vector elements using signals including but not limited to high frequency stimulation, such as with stimulation frequency F>=50 Hz, F>=100 Hz, F>=1,000 Hz, F>=10,000 Hz, F>=100,000 Hz, F>=1 MHz, F>=10 MHz, F>=100 MHz, F>=1 GHz, F>=10 GHZ.

Optimization of ligand specific signal generation

Stimulation or augmentation of one or multiple autonomic vector elements using signals including but not limited to lower frequency stimulation, such as with stimulation frequency F<=30 Hz, F<=50 Hz, F<=100 Hz, F<=1,000 Hz, and other ranges is also taught in the present invention.

Use of more than one stimulation signal, for simplicity called a carrier signal, to establish a beat frequency to allow modulation, including stimulation and inhibition, at the frequency difference between the two carrier signals. This may be used for modulation (including stimulation and inhibition) of structures including but not limited to the brain, intracranial structures, cranial nerves, spinal cord, peripheral nerves, autonomic structures, autonomic nerves, splanchnic nerves, greater splanchnic nerves, lesser splanchnic nerves, sympathetic nerves, vagus nerves, celiac plexus, mesenteric plexus, nerves innervating the pancreas, nerves innervating the gastrointestinal tract, nerves innervating the gastric antrum, nerves innervating the gastric pylorus, any other structures listed in this application, any other neural structures in the body.

An array of such modulators as shown in FIG. 54 and FIG. 55 enables delivery of a vector modulation signal which may deliver differing magnitudes and phases of modulation to each autonomic vector element, allowing for precise control of autonomic vector and therefore precise control of physiological activity. Such stimulation includes selective stimulation of metabolic function, such as augmentation or inhibition of metabolic activity, with modulation, including inhibition or stimulation, of cardiac activity, thereby delivering a cardioprotective component of modulation.

Autonomic Vector Modulation utilizing Interference Frequency Vectors:

FIG. 56 and FIG. 57 demonstrate preferred embodiments of a noninvasive neuromodulatory configuration or implanted neuromodulatory configuration with some relevant anatomy. Multiple modalities are taught and exemplified. In this configuration, one preferred embodiment comprises modulators or neuromodulators 421 which emit a high frequency modulator carrier frequency (MCF) 466. In one preferred embodiment shown, Neuromodulators 421 are placed in communication with the skin 280 of the user. The present invention teaches the use of single and multimodality modulation, comprising energy forms including but not limited to electrical, optical, pressure, vibration, electrical, magnetic, electromagnetic, and others. In this novel vector array, a single or multiplicity of modulators or neuromodulators deliver a modulator carrier frequency (MCF) 466. Each modulator or neuromodulator may have a unique modulator carrier frequency (MCF) 466 element, which may be referred to as a modulator carrier frequency element (MCFᵢ) for that modulator or neuromodulator. For any pair of modulators i and j, respective modulator carrier frequency i (MCFᵢ) 466 and modulator carrier frequency j (MCFⱼ) 466 may be chosen such that the modulator interference frequency (MIF) 467 is the difference between the two:

MIF=MCFᵢ−MCFᵢ

The modulation carrier frequencies (MCFₐ, . . . , MCFᵢ, . . . , MCFⱼ, . . . , MCF_{N}) 466 may be chosen to be outside of the frequency ranges in which stimulation and inhibition of the target tissue occurs. For example, modulation carrier frequencies (MCF) 466 of 1 MHz may be used to accomplish this. Alternatively or additionally, modulation carrier frequencies (MCF) 466 of higher or lower may be used, including but not limited to 100 KHz-1 KHz, 1 KHz-10 KHz, 10 KHz-100 KHz, 100 KHz-1 MHz, 1 MHz-10 MHz, 10 MHz-100 MHZ, 100 MHz-1 GHz, 1 GHz-10 GHz, 10 GHZ-100 GHz, 100 GHz-1 THz, 1 THz-10 THz, 10 THz-100 THz.

Modulation carrier frequencies (MCF) 466 may be selected to generate a single or plurality of modulator interference frequency (MIF) 467, such that the modulator interference frequency (MIF) 467 are within a frequency range that generates at least one of stimulation and inhibition of the target tissue. For example, a modulator interference frequency (MIF) 467 of 30 Hz may be used to stimulate target tissue. For example, a modulator interference frequency (MIF) 467 of 180 Hz may be used to inhibit target tissue. Alternatively or additionally, a single or plurality of modulator interference frequency (MIF) 467 may be generated in at least one of the ranges 0-10 Hz, 10 Hz-100 Hz, 100 Hz-1 KHz, 1 KHz-10 KHz, 10 KHz-100 KHz, 100 KHz-1 MHz, 1 MHz-10 MHz, 10 MHz-100 MHz, 100 MHz-1 GHz, 1 GHz-10 GHz, and other frequency ranges, including but not limited to those listed elsewhere in this specification.

Modulator Interference Frequency (MIF) 467 is delivered to target structure 468, which may be at least one of Right Sympathetic Trunk 71, Left Sympathetic Trunk 72, Right Greater Splanchnic Nerve 73, Left Greater Splanchnic Nerve 74, Right Lesser Splanchnic Nerve 75, Left Lesser Splanchnic Nerve 76, Right Subdiaphragmatic Greater Splanchnic Nerve 78, Left Subdiaphragmatic Greater Splanchnic Nerve 79, Right Subdiaphragmatic Lesser Splanchnic Nerve 80, Left Subdiaphragmatic Lesser Splanchnic Nerve 81, Intercostal Nerve 69, Intercostal Nerve 70, Right Vagus Nerve 95, Left Vagus Nerve 96, Intermediolateral Nucleus 121, Spinal Cord White Matter 122, Ventral Spinal Root 124, Dorsal Spinal Root 125, Spinal Ganglion 126, Spinal Nerve 127, Spinal Nerve Anterior Ramus 128, Spinal Nerve Posterior Ramus 129, Grey Ramus Communicantes 130, White Ramus Communicantes 131, Sympathetic Trunk 132, Ventral Horn of Spinal Gray Matter 141, Dorsal Horn of Spinal Gray Matter 142, Spinal Cord 151, Celiac Plexus 154, Celiac Ganglion 155, Superior Mesenteric Plexus 156, Superior Mesenteric Ganglion 157, Renal Plexus 158, Renal Ganglion 159, Inferior Mesenteric Plexus 160, Iliac Plexus 161, Right Lumbar Sympathetic Ganglia 162, Left Lumbar Sympathetic Ganglia 163, Right Sacral Sympathetic Ganglia 164, Left Sacral Sympathetic Ganglia 165, Abdominal Neural Plexus 187, Abdominal Neural Ganglion 188, Right Lumbar Sympathetic Trunk 189, Left Lumbar Sympathetic Trunk 190, Right Cervical Plexus 237, Left Cervical Plexus 238, Superficial Cardiac Plexus 244, Deep Cardiac Plexus 245, Right Anterior Pulmonary Nerve 246, Left Anterior Pulmonary Nerve 247, Right Renal Nerve Branch 248, Left Renal Nerve Branch 249, Stomach 8, Gastric Fundus 9, Greater Curvature of Stomach 10, Pyloric Antrum 11, Gastric Pylorus 12, Duodenum 13, Lower Esophageal Sphincter 14, Esophagus 15, Cardiac Notch of Stomach 16, Lesser Curvature of Stomach 17, Nerve 35, Transected Nerve End 37, Vagus Nerve 47, Sympathetic Nerve Branch 48, Intercostal Nerve 69, Intercostal Nerve 70, and other structure.

In FIG. 56, preferred embodiment includes any single or multiplicity of modulation modalities, ad enumerated above. Field lines depicted in this figure may be most closely representative of sonic or ultrasound wave propagation and interference patterns; however, this is only representative of the broader invention taught in the instant application.

In FIG. 57, preferred embodiment includes any single or multiplicity of modulation modalities, ad enumerated above. Field lines depicted in this figure may be most closely representative of electrical field generation and interference patterns; however, this is only representative of the broader invention taught in the instant application.

Autonomic Vector Control System and Modulation Zone Focusing and Confinement:

In FIG. 56 and FIG. 57, at least one Neuromodulation Signal (NMS) 998 is generated, each of said Neuromodulation Signal (NMS) 998 comprising an element of Neuromodulation Signal Vector (NSV) 996, which is at least one of generated and controlled by Autonomic Vector Control System (AVCS) 465. Through appropriate selection of a single or multiplicity of modulator carrier frequency i (MCFᵢ) 466 and modulator carrier frequency j (MCFⱼ) 466, delivered by modulators i and j, respectively, a single or multiplicity of Modulator Interference Frequency k (MIFₖ) 467 may be generated. Modulator Interference Frequency k (MIFₖ) 467 may be chosen to have any desired neuromodulatory or modulatory effect, including but not limited to stimulation, potentiation, inhibition, attenuation, blockage, any other effect, and any combination thereof.

FIG. 57 further demonstrates a novel application of the novel Autonomic Vector Control System (AVCS) 465 which performs Autonomic Vector Modulation (AVM) 472, including at least one of Noninvasive Autonomic Vector Modulation 473, Minimally Invasive Autonomic Vector Modulation 474, and Invasive Autonomic Vector Modulation 475, using Neuromodulation Signal Vector (NSV) 996, which may be implemented as a vector of Modulator Interference Frequency k (MIFₖ) 467, which may comprise:
Modulator Interference Frequency k (MIFₖ) 467, for k=1 to m,

where m may be chosen to be 0 to 1,000, or 1 to 100, or 1 to 24, or 0 to 12, or 0 to 10, or 0 to 8, or other range as desired. The range of k−1 to m may be chosen to correspond to the vector size of the autonomic vector being modulated, or m may be chosen to be larger or smaller than the autonomic vector being modulated.

In FIG. 57, a subset of the Modulator Interference Frequency (MIF) 467 shown are labeled specifically; and to describe this set, as an example, the value for m=3, and the specific Modulator Interference Frequency (MIF) 467 implementations are shown below and may be chosen to have any desired neuromodulatory or modulatory effect, including but not limited to stimulation, activation, augmentation, inhibition, blocking, temporal modulation, or other function, patient controlled paradigm, continuous value, intermittent value, physiologically triggered values or signals, physiologically controlled values, including open-loop control, closed-loop control, feedforward control, feedback control, adaptive control, or other control paradigms:
469 Modulator Interference Frequency 1 (MIF1)
470 Modulator Interference Frequency 2 (MIF2)
471 Modulator Interference Frequency 3 (MIF3)

A single or multiplicity of paradigms may be applied in the selection of Modulator Interference Frequency k (MIFₖ) 467. Modulator Interference Frequency k (MIFₖ) 467 may be selected to comprise a Neuromodulation Signal Vector (NSV) 996, comprising elements as may be defined below:

Neuromodulation Signal (NMS) Vector=[NMSₖ], for k=1 to m Modulator Interference Frequency k (MIFₖ), for k=1 to m

modulation using a vector of k elements, where k represents the size of the vector and which may also represent the dimensionality of the Neuromodulation Signal (NMS) Vector for the case in which each of the elements are orthogonal or independent, which may or may not be the case for a particular application.

In this preferred embodiment, Neuromodulation Signal Vector (NSV) 996 comprises a single or multiplicity of elements, each element of which may be a single or multiplicity of Neuromodulation signal (NMS) 998.

For any pair of modulators i and j, respective modulator carrier frequency i (MCFᵢ) and modulator carrier frequency j (MCFⱼ) may be chosen such that the modulator interference frequency (MIF) 467 is the difference between the two:

MIF=MCFᵢ−MCFᵢ

One preferred embodiment includes any single or multiplicity of modulation modalities, as enumerated above. Field lines depicted in each of FIG. 56 and FIG. 57 may be most closely representative of electrical field generation and interference patterns; however, this invention also includes the use of interference fields using ultrasound energy, sonic energy, magnetic energy, electromagnetic energy, optical energy, radiofrequency energy, all energy forms disclosed elsewhere in the present invention and specification and other forms of energy and signals, is only representative of the broader invention taught in the instant application.

FIG. 57 shows one of many possible configurations of Autonomic Vector Control System (AVCS) 465; in this configuration, the fields may be generate parallel to the longitudinal axis of the target structure axons, such as the axons comprising a nerve, pathway, sympathetic trunk, splanchnic nerve, vagus nerve, somatic nerve, autonomic nerve, brain white matter tract, spinal cord pathway, any pathway or structure described in the present invention or specification, or other pathway. In one preferred embodiment, Autonomic Vector Control System (AVCS) 465 is oriented with fields parallel to the longitudinal axis of the sympathetic trunk (including but not limited to the Right Sympathetic Trunk 71 and the Light Sympathetic Trunk 72 and other autonomic and neural structures), and as such the Neuromodulation Signal Vector (NSV) 996 may couple with specific spinal nodes innervating the sympathetic trunk, providing several novel features that enhance safety and efficacy. The novel orientation of Neuromodulation Signal Vector (NSV) 996 which may be parallel to the axis of sympathetic trunk and other structures allows for lower threshold stimulation and therefore stronger selective coupling to this structure. The novel vector arrangement facilitates focal stimulation of individual autonomic nodes and the selective inhibition of other autonomic nodes, thereby fine tuning the activation and inhibition, creating a shaped window of activation or od inhibition or of a novel combination of activation and inhibition as a function of autonomic node.

Autonomic Vector Modulation utilizing Passive Implants:

Enhanced focality may be accomplished through the use of active or of passive implants which are powered internally or externally. The use of external power delivered through the skin to passive implants provides a greater degree of safety and both technical and regulatory simplicity in the implementation of the Autonomic Vector Control System (AVCS) 465. Micro-implants with passive elements including a diode and energy resonant and coupling elements such as an induction coil and a resistor and capacitor, or a subset or superset of these, may be used to receive energy through magnetic, indictive, electromagnetic, and other coupling mechanisms. These implants may then more focally deliver energy to specific autonomic nodes to provide a Neuromodulation Signal Vector (NSV) 996 with enhanced spatial precision.

Autonomic Vector Modulation utilizing Other Energy Modalities:

The Autonomic Vector Control System (AVCS) 465 may be implemented using any single or plurality of energy modalities without departing from the present invention, said energy modalities include but are not limited to electrical energy, magnetic energy, electromagnetic energy, ultrasonic energy, sonic energy, subsonic energy, vibrotactile energy, vibrational energy, optical energy, any other energy form described or listed in the present invention and/or specification, and other energy forms. Additionally, energy and/or signals may eb delivered percutaneously using transmission means that are conductive to that energy form, with as percutaneous wires, optical fibers, conductive gels, natural structures which provide conductivity, and other means.

Autonomic Vector Modulation utilizing Multimodality Modulation:

Modulation of autonomic vectors may be performed utilizing multiple modalities as taught in the present inventions. Combinations of modalities, including but not limited to at least one energy form disclosed or mentioned in the present invention, and all permutations thereof, are included in the present invention.

Multiple previous attempts by well-funded research groups have failed to demonstrate efficacy in metabolic control utilizing a variety of modulation modalities. One preferred embodiment teaches the use of multiple modalities to achieve an optimal balance of modulation of sympathetic pathways and parasympathetic pathways. By utilizing a vector of excitatory neuromodulation modalities to stimulate sympathetic cells, fibers, or pathways and by using a vector of inhibitory neuromodulation modalities to suppress or block parasympathetic fibers, selected components of the autonomic vector may be increased, including those specific to metabolism and metabolic rate or to other desired physiological functions taught in the present invention.

Similarly, by utilizing a vector of excitatory neuromodulation modalities to stimulate parasympathetic cells, fibers, or pathways and by using a vector of inhibitory neuromodulation modalities to suppress or block sympathetic fibers, selected components of the autonomic vector may be decreased, including those specific to metabolism and metabolic rate or to other desired physiological functions taught in the present invention. Furthermore, additional pathways, including somatic and somatovisceral pathways may be modulated separately or concurrently with Autonomic Vector Modulation 472 by Autonomic Vector Control System (AVCS) 465 to further augment efficacy and safety.

Target structure 468 may comprise any neural or physiological structure in the body464 including but not limited to any neural structure or other structure in the present invention. Additionally, target structure 468 may include brown adipose tissue (BAT) in any location including but not limited to the thorax, abdomen, neck, back, arms, or legs, supraclavicular regions, head, brain, spinal cord, peripheral nerves, and any other structure, and in any other location.

In one preferred embodiment, a combination of at least one modality including sonic, ultrasonic, electrical, direct current electrical, alternating current electrical, magnetic, and optical energy are delivered to the supraclavicular brown adipose tissue (BAT) 476 fat pads. Modulation may be delivered directly to the brown adipose tissue (BAT) 476 or indirectly via the innervating pathways, including but not limited to neural structures, hormonal pathways, adrenergic signals, epinephrine, norepinephrine, sympathetic nervous system, parasympathetic nervous system, autonomic nervous system, enteric nervous system, and other pathway.

In another preferred embodiment, a combination of at least one modality including sonic, ultrasonic, electrical, direct current electrical, alternating current electrical, magnetic, and optical energy are delivered to the white adipose tissue (WAT) 477. Modulation may be delivered directly to the white adipose tissue (WAT) 477 or indirectly via the innervating pathways, including but not limited to neural structures, hormonal pathways, adrenergic signals, epinephrine, norepinephrine, sympathetic nervous system, parasympathetic nervous system, autonomic nervous system, enteric nervous system, and other pathway.

In another preferred embodiment, a combination of at least one modality including sonic, ultrasonic, electrical, direct current electrical, alternating current electrical, magnetic, and optical energy are delivered to the mastoid region of the skull in order to modulate the vestibular nerve directly or indirectly via the innervating pathways, including but not limited to neural structures, sympathetic nervous system, parasympathetic nervous system, autonomic nervous system, sensory nervous system, motor nervous system, visceral afferent nervous system, visceral efferent nervous system, and other pathway.

On one preferred embodiment, a combination of at least one modality including sonic, ultrasonic, electrical, direct current electrical, alternating current electrical, magnetic, and optical energy are delivered to target structure 468 in a manner that induces plastic changes, such as neural plasticity, resulting in durable changes in physiology that persist beyond the duration of the neuromodulation signal. One advantage of this modality is that it permits the use of intermittent stimulation, which is less susceptible to habituation or tachyphylaxis, phenomena in which the physiological response taper down with successive stimuli. For example, by inducing at least one of plastic changes such as a reduction in parasympathetic nervous system activity and plastic changes such as an increase in sympathetic nervous system activity, a durable increase in autonomic index or an increase in specific components of the autonomic vector may be effected and which persist after termination of the neuromodulation signal. Conversely, by inducing at least one of plastic changes such as a reduction in sympathetic nervous system activity and plastic changes such as an increase in parasympathetic nervous system activity, a durable decrease in autonomic index or a decrease in specific components of the autonomic vector may be effected and which persist after termination of the neuromodulation signal.

Autonomic Vector Modulation: Representative Indications and Applications:

Autonomic Vector Modulation 472, as that taught in the present invention and performed by Autonomic Vector Control System (AVCS) 465, may be used to provide optimal and maximal efficacy and optimal and minimal side effects in the treatment of a multiplicity disorders, including but not limited to at least one of the prevention and treatment of: Cardiac Failure, right heart failure, left heart failure, congestive heart failure (CHF), systolic heart disease, diastolic heart disease, Takotsubo's cardiomyopathy, acute respiratory distress syndrome (ARDS), obesity, asthma, hypertension, hypotension, sepsis, inflammation, traumatic brain injury (TBI), necrotizing enterocolitis (NEC), dysautonomia, reduction of stress-induced disease processes, reduction of inflammation induced disease processes, and other neurological, autonomic, somatic, and other diseases and conditions.

Autonomic Vector Modulation 472, as that taught in the present invention and performed by Autonomic Vector Control System (AVCS) 465, may also be used for physiological training and performance enhancement, including cardiovascular training, weight loss, weight optimization, attention enhancement, cognitive enhancement, memory enhancement, relaxation, meditation, stress reduction, lifespan augmentation, lifespan extension, and other health promotion objectives.

Life extension: modulation of metabolic parameters, including but not limited to those taught in the present invention above and further specifically including autonomic and other physiologic modulation of heart rate, blood pressure, energy metabolism including cell respiration, glucose metabolism, lipid metabolism, oxygen consumption, carbon dioxide production, and other neural and physiological pathways are taught for slowing down the organism metabolic process I order to conserve energy and to extend lifespan.

Autonomic Vector Modulation: Tachvphylaxis Minimization:

Autonomic Vector Modulation 472, as that taught in the present invention and performed by Autonomic Vector Control System (AVCS) 465, may be used to provide temporally patterned modulation that takes advantage of coupling between autonomic nodes and other neural nodes in the nervous system network to alternate or partition in time or in space the neuromodulatory signal (NMS) 998 elements of Neuromodulation Signal Vector (NSV) 996 such that tachyphylaxis that would develop if a solely scalar signal delivered may be avoided or minimized by delivering a multiplicity of coordinated signals comprising Neuromodulation Signal Vector (NSV) 996. Neuromodulation Signal Vector (NSV) 996 may be generated by Autonomic Vector Control System (AVCS) 465 to perform Autonomic Vector Modulation 472 which delivers individual neuromodulatory signals (NMS) 998 which are of a lower amplitude, lower duty cycle, lower amplitude and lower duty cycle, and which dynamically adjust to maximize use of nodes robustly coupled to the desired physiological effect and minimize use of nodes less robustly coupled to the desired effect in order to dynamically maximize efficacy and safety, dynamically minimize side effects, and dynamically adjust to minimize tachyphylaxis.

Energy Field Orientation:

Application of electrical fields parallel to sympathetic nervous system structures is taught to optimize modulatory coupling between said neuromodulator and nervous system tissue. Such a configuration may allow for a decreased stimulation threshold, thereby improving coupling between neuromodulator and nervous system component. This is useful for axons, comprising non-myelinated fibers and myelinated fibers. Electrical fields may alternately be applied perpendicular to sympathetic nervous system structures.

Electrical fields may applied parallel to single or plurality of nervous system structures comprising fibers, tracts, nuclei, single or plurality of ganglia, prevertebral ganglia, paravertebral ganglia, spinal root, spinal nerve, grey ramus, white ramus, sympathetic trunk, sympathetic chain, splanchnic nerve, vagus nerve, peripheral nerve, cranial nerve, autonomic structure, sympathetic structure, parasympathetic structure, axons, dendrites, soma, cortex, cerebral structure, cerebellar structure, brainstem structure, or other structures.

Electrical fields may applied perpendicular to single or plurality of nervous system structures comprising fibers, tracts, nuclei, single or plurality of ganglia, prevertebral ganglia, paravertebral ganglia, spinal root, spinal nerve, grey ramus, white ramus, sympathetic trunk, sympathetic chain, splanchnic nerve, vagus nerve, peripheral nerve, cranial nerve, autonomic structure, sympathetic structure, parasympathetic structure, axons, dendrites, soma, cortex, cerebral structure, cerebellar structure, brainstem structure, or other structures.

Electrical fields may applied oblique to single or plurality of nervous system structures comprising fibers, tracts, nuclei, single or plurality of ganglia, prevertebral ganglia, paravertebral ganglia, spinal root, spinal nerve, grey ramus, white ramus, sympathetic trunk, sympathetic chain, splanchnic nerve, vagus nerve, peripheral nerve, cranial nerve, autonomic structure, sympathetic structure, parasympathetic structure, axons, dendrites, soma, cortex, cerebral structure, cerebellar structure, brainstem structure, or other structures.

FIG. 54 depicts one preferred embodiment employing the noninvasive delivery of electrical energy to at least one of (1) diagnose a neurological or systemic or other condition, (2) screen for a physiological response to modulation of neural structure, and (3) to administer neuromodulatory signal as therapy.

Ablation:

Further embodiments of the present invention comprise neuromodulation of targets for which the modulating effect is fully reversible, partially reversible, and irreversible.

Further embodiments of the present invention comprise neuromodulation of targets for which the modulating effect is immediately reversible, reversible in a delayed time manner, reversible with gradual return of function, reversible with graded return of function, reversible with return of function in a linear manner, reversible with return of function in a non-linear manner, reversible with return of function in an exponential manner, reversible with return of function in a continuous manner, reversible with return of function in a discrete manner, reversible with return of function in a piecewise continuous manner, reversible with return of function over a predetermined tome course, reversible with return of function over a random tie course, reversible with return of function over a another time course.

Trialing:

Electrical neuromodulation as taught herein may be used for at least one of diagnosis, trialing, and therapy. In one embodiment, neuromodulation may be used to assess the system response of the physiology of the patient or user to assess information including diagnostic information. Trialing may then be performed to assess whether and to what degree the said patient or user will respond to the therapy and therefore is likely to be a responder. Therapeutic neuromodulation may then be performed to deliver at least one of acute, subacute, intermittent, periodic, sporadic, and chronic therapy.

At least one of trialing and therapy may be done with percutaneous single contact or multiple contact electrode catheters or other electrode configurations, and theses may be done with electrode catheters which are tunneled in the subcutaneous space or which follow a direct linear percutaneous trajectory. Said electrode catheters may be configured in any of several ways without departing form the present invention, and these may comprise flexible or rigid catheters, catheters with omnidirectional electrodes, catheters with unidirectional electrodes, catheters with directional electrodes, electrode wires with pattered insulation, electrode wires with insulation along the length with the exception of the region of the exposed electrode at the tip or along the length or other location, or other configuration.

Calibration:

Neuromodulation may be performed in a closed loop manner for a variety of applications including but not limited to the validation of the anatomical target. Neuromodulation may be performed using any of the modalities taught herein as well as other modalities, including those known to those versed in the art. For example, at least one of thermal, radiofrequency, ultrasonic, high frequency ultrasonic, sonic, and other modalities may be used to reversible stimulate or inhibit a prospective target. The physiological response may then be sensed directly or indirectly, and the degree of response fed into a control law which may then be used to adjust the degree of neuromodulation to assess the physiological response and then if desired to perform partially or completely irreversible neuromodulation.

Noninvasive Modulation of Neural and Other Structures:

Transcutaneous modulation of neural structures is taught for the treatment of conditions and diseases comprising but not limited to those listed and referenced in the present and related applications.

In FIG. 45, a preferred embodiment for noninvasive neuromodulation of neural structures is shown. Neural structures shown comprise but are not limited to superficial cardiac plexus 244, deep cardiac plexus 245.

Energy forms include but are not limited to electrical, vibratory, subsonic, sonic, ultrasonic, high frequency ultrasonic, optical, magnetic, electromagnetic, pressure wave, kinetic, thermal including heating and or cooling, other energy forms in the present invention and specification, and other energy forms.

Autonomic Vector Control System—System Configurations and Considerations:

Preferred embodiments include a single or a plurality of neuromodulators configured to modulate a singularity or a plurality of neural pathways, including but not limited to autonomic pathways, comprising sympathetic pathways, parasympathetic pathways, sympathetic and parasympathetic pathways, a combination of including. By appropriately modulating an autonomic vector, autonomic indices corresponding to metabolic subvector components may be down modulated while maintaining an overall balance in autonomic activity. This may be applied to the slowing down of metabolic processes that correlate with subcellular genetic and organelle processes that correlate with ageing at the cellular level. Through chronic application of metabolic subvector inhibition, using at least one of implanted, percutaneous, noninvasive, and other neuromodulators, one can reduce metabolic stresses at the cellular and organ level, thereby contributing to longevity at the organism level.

A preferred embodiment includes the use of an external nonimplanted power supply in communication with an implanted passive electronic circuit and neuromodulator. This allows for simpler product design and regulatory approval processes while facilitating easier programming and updating of control parameters, which may be embodies in the external controller. This external power and internal passive circuit and neuromodulator configuration may be applied to one or any of the neuromodulator locations, indications, proposes, functions, functionalities, form factors, implantation procedures, array configurations, vector configurations, or any other aspect of the instant invention and any of the inventions included by reference without departing from the present invention. An external power supply may transmit power to the passive implant using energy modalities including but not limited to tactile energy, including light touch, pressure, vibration, thermal energy, including hot and cold, as well as constant or variable temperatures, electrical energy, including direct current, alternating current, interferential signals, magnetic stimulation, including static magnetic fields, time varying magnetic fields, alternating magnetic fields, pulsed magnetic fields, transcranial magnetic stimulation (TMS) signals, transcutaneous magnetic stimulation signals, electromagnetic stimulation, including stimulation with radiofrequency energy, very low frequency signals, extremely low frequency (ELF) signals, sub-ELF signals, radiofrequency signals, microwave signals, infrared light signals, visible light signals, ultraviolet light signals X-Ray signals, gamma ray signals, and other electromagnetic frequency bands, optical stimulation, including visible light, infrared light, ultraviolet light, and other stimulation with photons of these and other wavelengths, pressure wave signals including ultrasonic, high frequency ultrasonic, sonic, subsonic, and other pressure wave frequencies, and other energy forms. In one preferred embodiment, an external magnetic signal may be transmitted through the skin to an implanted passive device which employs an antenna and a passive network to convert the alternating magnetic field into electrical energy for use in stimulating neural tissue. At least one of a half-wave bridge and a full wave bridge may be included in the passive implant to rectify the alternating signals in order to create a DC signal for powering on board circuitry or for delivering a signal which may have a DC component. In one preferred embodiment, a charge-balanced alternating signal is delivered to the neural tissues.

### CONCLUSION

It will be appreciated by those skilled in the art that while the invention has been described above in connection with the particular embodiments and examples, the invention is not necessarily so limited, and that numerous other embodiments, examples uses, modifications, and departures from the embodiments, examples, and uses are intended to be encompassed by the claims attached hereto The entire disclosure of each patent and publication cited herein is incorporated by reference, as if each such patent or publication were individually incorporated by reference herein.

1. An apparatus for use in at least one of enhancing health and treating disease in an organism by performing vector modulation of the autonomic nervous system comprising at least one autonomic element modulator, comprising:
A. a pulse generator, in communication with a neuromodulator; and
B. the neuromodulator, which modulates the activity of a vector element of the autonomic nervous system, in said organism.

2. The apparatus of claim 1 further comprising a controller.

3. The apparatus of claim 1 further comprising at least one additional neuromodulator.

4. The apparatus of claim 1, wherein a multiplicity of neuromodulatory signals are delivered to at least one node in an autonomic vector.

5. The apparatus of claim 1, wherein a multiplicity of independent neuromodulatory signals are delivered to at least one node in an autonomic vector.

6. The apparatus of claim 1, wherein a multiplicity of neuromodulatory signals are delivered to more than one node in an autonomic vector.

7. The apparatus of claim 6, wherein at least one neuromodulatory signal is stimulatory.

8. The apparatus of claim 6, wherein at least one neuromodulatory signal is inhibitory.

9. The apparatus of claim 6, wherein at least one neuromodulatory signal is stimulatory and at least one neuromodulatory signal is inhibitory.

10. The apparatus of claim 6, further comprising a sensor.

11. The apparatus of claim 6, further comprising a multiplicity of sensors.

12. The apparatus of claim 6, wherein said sensor is configured to sense at least one physiological signal representative of disease state.

13. The apparatus of claim 6, wherein said sensor is configured to sense at least one physiological signal representative of response to therapy.

14. The apparatus of claim 6, wherein said sensor is configured to sense at least one physiological signal representative of side effects.

15. The apparatus of claim 6, wherein said sensor is configured to sense at least one physiological signal representative of side effects.

16. The apparatus of claim 6, wherein said controller is configured to calculate an autonomic state.

17. The apparatus of claim 6, wherein said controller is configured to calculate a neuromodulatory vector which is a function of the autonomic state.

18. The apparatus of claim 17, wherein said controller is configured to calculate a neuromodulatory vector which at least one of maximizes efficacy and minimizes side effects.

19. The apparatus of claim 17, wherein said controller is configured to calculate an optimal neuromodulatory vector which maximizes efficacy and minimizes side effects.

20. The apparatus of claim 19, wherein said apparatus is configured to treat obesity.

21. The apparatus of claim 20, wherein said controller is configured to maximize weight loss and minimize cardiac side effects.

22. The apparatus of claim 20, wherein said sensor is configured to sense at least one physiological parameter representative of metabolic rate.

23. The apparatus of claim 20, wherein said sensor is configured to sense at least one physiological parameter representative of body weight.

24. The apparatus of claim 20, wherein said sensor is configured to sense at least one physiological parameter representative of visceral adipose tissue mass.

25. The apparatus of claim 20, wherein said sensor is configured to sense at least one physiological parameter representative of body mass index.

26. The apparatus of claim 20, wherein said sensor is configured to sense at least one physiological parameter representative of respiratory quotient.

27. The apparatus of claim 20, wherein said sensor is configured to sense at least one physiological parameter representative of autonomic index.

28. The apparatus of claim 20, wherein said sensor is configured to sense at least one physiological parameter representative of blood pressure.

29. The apparatus of claim 20, wherein said sensor is configured to sense at least one physiological parameter representative of heart rate.

30. The apparatus of claim 20, wherein said sensor is configured to sense at least one physiological parameter representative of heart rate.

31. The apparatus of claim 21, wherein said controller is configured to calculate an optimal neuromodulatory vector as a function of at least one physiological parameter.

32. The apparatus of claim 31, wherein said controller is configured to calculate an optimal neuromodulatory vector as a function of at least one physiological parameter, comprising at least one of metabolic rate, body weight, visceral adipose tissue mass, body mass index, respiratory quotient, autonomic index, blood pressure, heart rate, and heart rate variability.

33. The apparatus of claim 19, wherein said apparatus is configured to treat asthma.

34. The apparatus of claim 33, wherein said apparatus is configured to optimally deliver autonomic vector modulation to increase bronchodilation while minimizing cardiac side effects

35. The apparatus of claim 19, wherein said apparatus is configured to treat hypertension.

36. The apparatus of claim 35, wherein said apparatus is configured to optimally deliver autonomic vector modulation to decrease blood pressure while maintaining circulation to at least one of brain, visceral organs, and extremities.

37. The apparatus of claim 19, wherein said apparatus is configured to treat shock.

38. The apparatus of claim 37, wherein said apparatus is configured to optimally deliver autonomic vector modulation to increase blood pressure while maintaining circulation to at least one of brain, visceral organs, and extremities.

39. The apparatus of claim 37, wherein said apparatus is configured to optimally deliver autonomic vector modulation to increase blood pressure while minimizing risk of cardiac strain, risk of myocardial infarction, risk of cardiac arrhythmia.

40. The apparatus of claim 19, wherein said apparatus is configured to treat irritable bowel disease.

41. The apparatus of claim 40, wherein said apparatus is configured to optimally deliver autonomic vector modulation to decrease gastrointestinal symptoms, including at least one of motility, secretions, and discomfort, while minimizing at least one of cardiac side effects and vascular side effects.

42. The apparatus of claim 19, wherein said apparatus is configured to treat disease involving the brain.

43. The apparatus of claim 42, wherein said apparatus is configured to optimally deliver autonomic vector modulation to maximize treatment efficacy, while minimizing at treatment side effects.

44. The apparatus of claim 19, wherein said apparatus is configured to treat disease involving the spinal cord.

45. The apparatus of claim 44, wherein said apparatus is configured to optimally deliver autonomic vector modulation to maximize treatment efficacy, while minimizing at treatment side effects.

46. The apparatus of claim 19, wherein said apparatus is configured to treat disease involving the peripheral nerves.

47. The apparatus of claim 46, wherein said apparatus is configured to optimally deliver autonomic vector modulation to maximize treatment efficacy, while minimizing at treatment side effects.

48. The apparatus of claim 19, wherein said apparatus is configured to treat disease involving the autonomic nervous system

49. The apparatus of claim 42, wherein said apparatus is configured to optimally deliver autonomic vector modulation to maximize treatment efficacy, while minimizing at treatment side effects.

50. The apparatus of claim 19, wherein said apparatus is configured to treat disease involving the sympathetic nervous system

51. The apparatus of claim 50, wherein said apparatus is configured to optimally deliver autonomic vector modulation to maximize treatment efficacy, while minimizing at treatment side effects.

52. The apparatus of claim 19, wherein said apparatus is configured to treat disease involving the parasympathetic nervous system

53. The apparatus of claim 52, wherein said apparatus is configured to optimally deliver autonomic vector modulation to maximize treatment efficacy, while minimizing at treatment side effects.

54. The apparatus of claim 19, wherein said apparatus is configured to treat disease involving the enteric nervous system.

55. The apparatus of claim 54, wherein said apparatus is configured to optimally deliver autonomic vector modulation to maximize treatment efficacy, while minimizing at treatment side effects.

56. The apparatus of claim 19, wherein said apparatus is configured to treat adult respiratory distress syndrome.

57. The apparatus of claim 54, wherein said apparatus is configured to optimally deliver autonomic vector modulation to maximize treatment efficacy, while minimizing at treatment side effects.

58. The apparatus of claim 19, wherein said apparatus is configured to treat Takotsubo's cardiomyopathy.

59. The apparatus of claim 54, wherein said apparatus is configured to optimally deliver autonomic vector modulation to maximize treatment efficacy, while minimizing at treatment side effects.
